SEQUENCE LISTING (28 sequences)

SEQ ID NO: 1 - DNA, 22 bp, synthetic construct
  misc_feature 1..22: note = Single strand DNA oligonucleotide
  source 1..22: mol_type = other DNA
agtcaaggccgagaatgggaag

SEQ ID NO: 2 - DNA, 22 bp, synthetic construct
  misc_feature 1..22: note = Single strand DNA oligonucleotide
  source 1..22: mol_type = other DNA
aagcagttggtggtgcaggatg

SEQ ID NO: 3 - DNA, 21 bp, synthetic construct
  misc_feature 1..21: note = Single strand DNA oligonucleotide
  source 1..21: mol_type = other DNA
agaggatcaccttggggtaca

SEQ ID NO: 4 - DNA, 20 bp, synthetic construct
  misc_feature 1..20: note = Single strand DNA oligonucleotide
  source 1..20: mol_type = other DNA
cgaagcgacagatggtggtc

SEQ ID NO: 5 - DNA, 20 bp, synthetic construct
  misc_feature 1..20: note = Single strand DNA oligonucleotide
  source 1..20: mol_type = other DNA
ctcaagtcctgaggctgaca

SEQ ID NO: 6 - DNA, 20 bp, synthetic construct
  misc_feature 1..20: note = Single strand DNA oligonucleotide
  source 1..20: mol_type = other DNA
tgaaacctgtccttgagtgc

SEQ ID NO: 7 - DNA, 23 bp, synthetic construct
  misc_feature 1..23: note = Single strand DNA oligonucleotide
  source 1..23: mol_type = other DNA
tagagctagactccgggcgatga

SEQ ID NO: 8 - DNA, 23 bp, synthetic construct
  misc_feature 1..23: note = Single strand DNA oligonucleotide
  source 1..23: mol_type = other DNA
ttgccttaaacaagaccacgaaa

SEQ ID NO: 9 - DNA, 20 bp, synthetic construct
  misc_feature 1..20: note = Single strand DNA oligonucleotide
  source 1..20: mol_type = other DNA
gcacacctgcgaactcacac

SEQ ID NO: 10 - DNA, 21 bp, synthetic construct
  misc_feature 1..21: note = Single strand DNA oligonucleotide
  source 1..21: mol_type = other DNA
ccgtcccagtcacagtggtaa

SEQ ID NO: 11 - DNA, 22 bp, synthetic construct
  misc_feature 1..22: note = Single strand DNA oligonucleotide
  source 1..22: mol_type = other DNA
gcgaaacctgtgcgagtggatg

SEQ ID NO: 12 - DNA, 28 bp, synthetic construct
  misc_feature 1..28: note = Single strand DNA oligonucleotide
  source 1..28: mol_type = other DNA
cggtatttgtcttttgtcctggttttca

SEQ ID NO: 13 - DNA, 22 bp, synthetic construct
  misc_feature 1..22: note = Single strand DNA oligonucleotide
  source 1..22: mol_type = other DNA
cacaagatgaacggcatcaacc

SEQ ID NO: 14 - DNA, 18 bp, synthetic construct
  misc_feature 1..18: note = Single strand DNA oligonucleotide
  source 1..18: mol_type = other DNA
cagcgtggtggtagtctg

SEQ ID NO: 15 - DNA, 26 bp, synthetic construct
  misc_feature 1..26: note = Single strand DNA oligonucleotide
  source 1..26: mol_type = other DNA
cttgcgggctctatatgaaactccat

SEQ ID NO: 16 - DNA, 32 bp, synthetic construct
  misc_feature 1..32: note = Single strand DNA oligonucleotide
  source 1..32: mol_type = other DNA
tagaagaagaggaagtaggagtcatagggaca

SEQ ID NO: 17 - DNA, 26 bp, synthetic construct
  misc_feature 1..26: note = Single strand DNA oligonucleotide
  source 1..26: mol_type = other DNA
ctgtgactgcctaccagaatgaggag

SEQ ID NO: 18 - DNA, 25 bp, synthetic construct
  misc_feature 1..25: note = Single strand DNA oligonucleotide
  source 1..25: mol_type = other DNA
ggtcgtttctttctttggcatcaag

SEQ ID NO: 19 - DNA, 20 bp, synthetic construct
  misc_feature 1..20: note = Single strand DNA oligonucleotide
  source 1..20: mol_type = other DNA
cttcgggtatggacttgctg

SEQ ID NO: 20 - DNA, 22 bp, synthetic construct
  misc_feature 1..22: note = Single strand DNA oligonucleotide
  source 1..22: mol_type = other DNA
cctcatgaagatgtctgggtac

SEQ ID NO: 21 - DNA, 23 bp, synthetic construct
  misc_feature 1..23: note = Single strand DNA oligonucleotide
  source 1..23: mol_type = other DNA
caaagtcaatggctcccacgaag

SEQ ID NO: 22 - DNA, 27 bp, synthetic construct
  misc_feature 1..27: note = Single strand DNA oligonucleotide
  source 1..27: mol_type = other DNA
ctacaatcccctgagacacagcaaata

SEQ ID NO: 23 - DNA, 24 bp, synthetic construct
  misc_feature 1..24: note = CRISPR sequences
  source 1..24: mol_type = other DNA
caccgggagaagcacttctcggtc

SEQ ID NO: 24 - DNA, 20 bp, synthetic construct
  misc_feature 1..20: note = CRISPR sequences
  source 1..20: mol_type = other DNA
atgtagagctcctcgccggc

SEQ ID NO: 25 - DNA, 24 bp, synthetic construct
  misc_feature 1..24: note = CRISPR sequences
  source 1..24: mol_type = other DNA
caccgtggtgtctttgtcaacggg

SEQ ID NO: 26 - DNA, 20 bp, synthetic construct
  misc_feature 1..20: note = CRISPR sequences
  source 1..20: mol_type = other DNA
acacttactgttctgcatgc

SEQ ID NO: 27 - DNA, 25 bp, synthetic construct
  misc_feature 1..25: note = CRISPR sequences
  source 1..25: mol_type = other DNA
caccgagccaccgatgccttccgat

SEQ ID NO: 28 - DNA, 20 bp, synthetic construct
  misc_feature 1..20: note = CRISPR sequences
  source 1..20: mol_type = other DNA
gccgctgtccgctacccagg

(19) United States
(12) Patent Application Publication
(10) Pub. No.: US 2024/0026262 A1
(43) Pub. Date: Jan. 25, 2024

(54) METHODS AND DEVICES FOR EX-UTERO MOUSE EMBRYONIC DEVELOPMENT

(71) Applicant: Yeda Research and Development Co. Ltd., Rehovot (IL)

(72) Inventor: Yaqub HANNA, Tel Aviv-Yafo (IL)

(73) Assignee: Yeda Research and Development Co. Ltd., Rehovot (IL)

(21) Appl. No.: 18/369,233

(22) Filed: Sep. 18, 2023

Related U.S. Application Data
(63) Continuation of application No. PCT/IL2022/050294, filed on Mar. 15, 2022.

(30) Foreign Application Priority Data
Mar. 16, 2021 (IL) 281561

(51) Int. Cl. C12M 3/00; C12M 3/04; C12M 1/107; C12M 1/34; C12M 1/36; C12M 1/00

(52) U.S. Cl. CPC C12M 21/08; C12M 27/10; C12M 23/36; C12M 41/34; C12M 41/48; C12M 29/20

(57) ABSTRACT

Methods and devices for ex-utero mouse embryonic development are provided. Accordingly, there is provided a method of ex-utero culturing a mouse embryo at a zygote stage under conditions that allow developments of the embryo to organogenesis or any developmental stage therein-between. Also provided a fetal incubation system, and methods of using same.

### RELATED APPLICATIONS

This application is a Continuation of PCT Patent Application No. PCT/IL2022/050294 having International filing date of Mar. 15, 2022, which claims the benefit of priority of Israel Patent Application No. 281561 filed on Mar. 16, 2021. The contents of the above applications are all incorporated by reference as if fully set forth herein in their entirety.

### SEQUENCE LISTING STATEMENT

The XML file, entitled 97526SequenceListing.xml, created on Sep. 14, 2023, comprising 35,667 bytes, submitted concurrently with the filing of this application is incorporated herein by reference.

### FIELD AND BACKGROUND OF THE INVENTION

The present invention, in some embodiments thereof, relates to methods and devices for ex-utero mouse embryonic development.

Understanding the developmental processes leading to the formation of tissues represents one of the fundamental questions in developmental biology. In mammals, this process takes place after the embryo implants into the uterus. The intrauterine confinement of developing embryos has limited the study of post-implantation embryogenesis, due to the inability to observe, transfer and manipulate living embryos at these stages. While mouse embryos are consistently cultured through pre- and peri-implantation development [Bedzhov, I. & Zernicka-Goetz, M. Cell (2014); White, M. D. et al. Cell 165, 75-87 (2016)], establishing culture conditions sustaining proper long-term development of post-implanted mouse embryos outside the uterine environment remains challenging.

A number of culture techniques have been proposed over the years since the 1930s by culturing the embryos in conventional static conditions, in rotating bottles on a drum (referred to as “roller culture systems”) or on circulator platforms [e.g. Nicholas, J. S. & Rudnick, D. Proc. Natl. Acad. Sci. U.S.A. 20, 656-8 (1934); New, D. A. T. & Stein, K. F. Nature 199, 297-299 (1963); New, D. A. T., J. Reprod. Fertil. (1973); New, D. A. T. Development 17, (1967); New, D. A. T. Biol. Rev. 53, 81-122 (1978); Ellis-hutchings. R. G. & A, E. W. C. Whole Embryo Culture: A New Technique That Enabled Decades of Mechanistic Discoveries. 312, 304-312 2010); McDole, K. et al. Cell 175, 859-876.e33 (2018); US Patent Application Publication No. US20090304639; EP Patent No. EP2014316; UK Patent Application Publication No. GB2517194; JP Patent Application Publication No. JP2020501534; and International Patent Application Publication Nos. WO2000017326 and WO2002059276]. However, these platforms remain highly inefficient for normal embryo survival and are limited to short periods of time, as the embryos begin to display developmental anomalies as early as 24 hours following culture initiation. Thus, stable and efficient protocols for extended culturing of pre-gastrulating mouse embryos all the way until advanced organogenesis stages are still lacking. Further, the process of gastrulation has never been authentically captured ex utero in any mammalian in its entirety and normally. The entire process of organogenesis has not been continuously and normally captured in any mammalian embryo so far. Subsequently, the processes of gastrulation and organogenesis have not been continuously captured ex utero in a combined matter while yielding normal embryos.

### SUMMARY OF THE INVENTION

According to an aspect of some embodiments of the present invention there is provided a method of ex-utero culturing a mouse embryo, the method comprising culturing a mouse embryo at a late gastrulation stage in a dynamic culture under conditions that allow development of the embryo to a hind limb formation stage, wherein the conditions comprise a hyperbaric pressure of more than 5 and less than 10.2 pounds per square inch (psi); an atmosphere comprising increasing oxygen concentrations throughout the culturing starting from 5% up to 15-40%; and a medium comprising at least 30% serum, wherein the serum comprises rat serum and human serum, and a base medium comprising at least 1 mg/ml glucose up to an early somite stage and at least 3 mg/ml glucose when the embryo reaches the early somite stage.

According to some embodiments of the invention, the culturing is effected for about 4 days.

According to some embodiments of the invention, the culturing is from embryonic day (E)7.5 to E11-11.5.

According to some embodiments of the invention, the increasing is effected every 20-28 hours of the culturing.

According to an aspect of some embodiments of the present invention there is provided a method of ex-utero culturing a mouse embryo, the method comprising culturing a mouse embryo at a post implantation pre gastrulation to early gastrulation stage in a static culture under conditions that allow development of the embryo to an early somite stage, wherein the conditions comprise an atmosphere comprising 15-40% oxygen; and a medium comprising at least 30% serum, wherein the serum comprises rat serum and human serum, and a base medium comprising at least 1 mg/ml glucose.

According to some embodiments of the invention, optionally, the medium further comprises knockout serum replacement (KSR) in addition to the rat serum and the human serum.

According to some embodiments of the invention, optionally, the KSR partially replaces one of either the human serum, the rat serum or partially replaces a quantity of both.

According to some embodiments of the invention, the culturing is effected for 2-3 days.

According to some embodiments of the invention, the culturing is from embryonic day (E)5.5-6.5 to E8.5.

According to an aspect of some embodiments of the present invention there is provided a method of ex-utero culturing a mouse embryo, the method comprising culturing a mouse embryo at an implanting blastocyst stage in a static culture under conditions that allow development of the embryo to a post implantation pre gastrulation stage, wherein the conditions comprise an atmosphere comprising 15-40% oxygen; and a medium comprising 15-75% serum and a base medium comprising Insulin-Transferrin-Selenium-Ethanolamine (ITS-X), progesterone, sodium lactate and 3,3′,5-Triiodo-L-thyronine (T3).

According to some embodiments of the invention, the base medium further comprises N2 and/or B27 supplements.

According to some embodiments of the invention, the conditions comprise N2 and/or B27 in the base medium following 1-2 days of the culturing.

According to some embodiments of the invention, the serum comprises a bovine serum.

According to some embodiments of the invention, the serum comprises a human serum.

According to some embodiments of the invention, the 15-75% serum comprises 20-30% serum.

According to some embodiments of the invention, the base medium further comprises at least 1 mg/ml glucose.

According to some embodiments of the invention, the base medium further comprises (3-estradiol and/or N-acetyl-L-cysteine.

According to some embodiments of the invention, the culturing is effected for about 3 days.

According to some embodiments of the invention, the culturing is from embryonic day (E)4.5 to E5.5.

According to an aspect of some embodiments of the present invention there is provided a method of ex-utero culturing a mouse embryo, the method comprising:
(a) culturing a mouse embryo at a post implantation pre gastrulation to early gastrulation stage in a static culture under a first set of conditions that allow development of the embryo to an early somite stage, wherein the first set of conditions comprise an atmosphere comprising 15-40% oxygen; and a first medium comprising at least 30% serum, wherein the serum comprises rat serum and human serum, and a base medium comprising at least 1 mg/ml glucose, so as to obtain an embryo of an early somite stage; and
(b) culturing the embryo of the early somite stage in a dynamic culture under a second set of conditions that allow development of the embryo to a hind limb formation stage, wherein the second set of conditions comprise a hyperbaric pressure of more than 5 and less than 10.2 pounds per square inch (psi); an atmosphere comprising 15-40% oxygen; and a second medium comprising at least 30% serum, wherein the serum comprises rat serum and human serum, and a base medium comprising at least 3 mg/ml glucose.

According to some embodiments of the invention, optionally, the medium further comprises knockout serum replacement (KSR) in addition to the rat serum and the human serum.

According to some embodiments of the invention, optionally, the KSR partially replaces one of either the human serum, the rat serum or partially replaces a quantity of both.

According to some embodiments of the invention, the (a) is effected for 2-3 days.

According to some embodiments of the invention, the (b) is effected for about 3 days.

According to some embodiments of the invention, the culturing is from embryonic day (E)5.5-6.5 to E11-11.5.

According to an aspect of some embodiments of the present invention there is provided a method of ex-utero culturing a mouse embryo, the method comprising:
(a) culturing a mouse embryo at an implanting blastocyst stage according to the method so as to obtain the embryo of the post implantation pre gastrulation stage; and
(b) culturing the embryo of the post implantation pre gastrulation stage in a static culture under a second set of conditions that allow development of the embryo to a late gastrulation stage, wherein the second set of conditions comprise an atmosphere comprising 15-40% oxygen; and a second medium comprising at least 30% serum, wherein the serum comprises rat serum and human serum, and a base medium comprising at least 1 mg/ml glucose.

According to some embodiments of the invention, optionally, the medium further comprises knockout serum replacement (KSR) in addition to the rat serum and the human serum.

According to some embodiments of the invention, optionally, the KSR partially replaces one of either the human serum, the rat serum or partially replaces a quantity of both.

According to some embodiments of the invention, the (b) is effected for about 2 days.

According to some embodiments of the invention, the culturing is from embryonic day (E)4.5 to E7.5.

According to an aspect of some embodiments of the present invention there is provided a method of ex-utero culturing a mouse embryo, the method comprising culturing mouse embryo at an implanting blastocyst stage according to the method so as to obtain the embryo of the late gastrulation stage; and
(c) culturing the embryo of the late gastrulation stage in a dynamic culture under a third set of conditions that allow development of the embryo to an early somite stage, wherein the third set of conditions comprise an atmosphere comprising 15-40% oxygen; and the second medium.

According to some embodiments of the invention, the (c) is effected for about 1 day.

According to some embodiments of the invention, the culturing is from embryonic day (E)4.5 to E8.5.

According to an aspect of some embodiments of the present invention there is provided a method of ex-utero culturing a mouse embryo, the method comprising culturing mouse embryo at an implanting blastocyst stage according to the method so as to obtain the embryo of the early somite stage; and
(d) culturing the embryo of the early somite stage in a dynamic culture under a fourth set of conditions that allow development of the embryo to a hind limb formation stage, wherein the fourth set of conditions comprise a hyperbaric pressure of more than 5 and less than 10.2 pounds per square inch (psi); an atmosphere comprising 15-40% oxygen; and the second medium comprising at least 3 mg/ml glucose.

According to some embodiments of the invention, the (d) is effected for about 3 days.

According to some embodiments of the invention, the culturing is from embryonic day (E)4.5 to E11-11.5.

According to some embodiments of the invention, the at least 30% serum comprises at least 50% serum.

According to some embodiments of the invention, the at least 30% serum comprises 70-80% serum.

According to some embodiments of the invention, a ratio between the rat serum and the human serum is between 1:1-3:1.

According to some embodiments of the invention, a ratio between the serum and the base medium is between 1:1-5:1.

According to some embodiments of the invention, optionally, the medium further comprises knockout serum replacement (KSR) in addition to the rat serum and the human serum.

According to some embodiments of the invention, optionally, the KSR partially replaces one of either the human serum, the rat serum or partially replaces a quantity of both.

According to some embodiments of the invention, the dynamic culture is a roller culture.

According to some embodiments of the invention, the dynamic culture is a shaker culture.

According to some embodiments of the invention, the conditions comprise replacement of at least half of the medium every 20-28 hours of the culturing.

According to some embodiments of the invention, the glucose is provided in the medium in increasing concentrations throughout the culturing.

According to some embodiments of the invention, the increasing is effected every 20-28 hours.

According to some embodiments of the invention, the 15-40% oxygen comprises 19-23% oxygen.

According to some embodiments of the invention, the hyperbaric pressure is 6-7 psi.

According to an aspect of some embodiments of the present invention there is provided a fetal incubation system, comprising:
a. a gas controller, configured for providing a plurality of gases to at least one incubator;
b. at least one incubator comprising a rotating module inside of said at least one incubator; rotating module comprising one or more vials comprising said at least one embryo; wherein the system comprises one or more buffers for the plurality of gases being provided to the rotating module inside of the at least one incubator.

According to some embodiments of the invention, one of the one or more buffers is a gas mixing box for mixing the plurality of gases before being provided to the at least one incubator.

According to some embodiments of the invention, the gas controller comprises one or more specific gas controllers for individually control flow of specific one or more gases.

According to some embodiments of the invention, the gas controller comprises one or more electric valves for allowing flowing of the specific one or more gases.

According to some embodiments of the invention, the one or more specific gas controllers control activation and deactivation of the one or more electric valves.

According to some embodiments of the invention, the gas controller comprises a vacuum pump for extracting mixed gases from the gas mixing box.

According to some embodiments of the invention, the gas controller comprises a pressure pump in connection with the vacuum pump for providing the mixed gases to the system at hyperbaric pressures.

According to some embodiments of the invention, the pressure pump provide gases at pressures of from about 0.1 psi to about 20 psi.

According to some embodiments of the invention, the gas mixing box comprises one or more gas sensors.

According to some embodiments of the invention, the one or more gas sensors provide information to the one or more specific gas controllers.

According to some embodiments of the invention, the one or more specific gas controllers control activation and deactivation of the one or more electric valves according to the information received by the one or more gas sensors.

According to some embodiments of the invention, the gas mixing box comprises a mixer blower for mixing the plurality of gases in the gas mixing box.

According to some embodiments of the invention, the incubator comprises a unidirectional valve connected to the pressure pump.

According to some embodiments of the invention, the incubator comprises a humidifier connected to the unidirectional valve for humidifying the mixed gases.

According to some embodiments of the invention, the humidifier comprises a container with at least one liquid.

According to some embodiments of the invention, the at least one liquid is water.

According to some embodiments of the invention, the fetal incubation system further comprising a humidifier, which also functions as one of the buffers for the plurality of gases.

According to some embodiments of the invention, the rotational module comprises a rotational drum comprising one or more vials; the rotational drum connected to the humidifier.

According to some embodiments of the invention, the incubator comprises an outlet bottle for gases.

According to some embodiments of the invention, the outlet bottle for gases comprises a container with at least one liquid.

According to some embodiments of the invention, the at least one liquid is water.

According to some embodiments of the invention, the outlet bottle for gases functions as one of the buffers for the plurality of gases.

According to some embodiments of the invention, the rotational drum provides mixed gases to each of the plurality of individual sample bottles individually.

According to some embodiments of the invention, the one or more buffers are configured to maintain a determined concentration of the plurality of gases and a determined hyperbaric level substantially constant.

According to an aspect of some embodiments of the present invention there is provided a method of incubating fetuses in an incubator, comprising:
a. flowing mixed gases at a determined concentration into a rotational module located inside the incubator;
b. flowing the mixed gases at a determined hyperbaric level;
c. maintaining the determined concentration and the determined hyperbaric level substantially constant.

According to some embodiments of the invention, achieving the mixed gases at the determined concentration, comprises:
a. setting desired concentrations of each individual gas of the mixed gases;
b. flowing the individual gases into a gas mixing box;
c. sensing when each of the individual gases reaches the desired concentration;
d. mixing the gases inside the gas mixing box.

According to some embodiments of the invention, the flowing the mixed gases into the incubator comprises extracting the mixed gases from the gas mixing box and delivering into the incubator.

According to some embodiments of the invention, achieving the determined hyperbaric level, comprises:
a. setting a desired hyperbaric level;
b. allowing access of the mixed gases to a pressure pump until the hyperbaric level is reached.

According to some embodiments of the invention, the maintaining comprises providing a plurality of sensors to the gas mixing box for monitoring the concentrations of the each individual gas.

According to some embodiments of the invention, the incubator comprises a rotational drum comprising individual sample bottles.

According to some embodiments of the invention, the maintaining comprises providing pressure stabilizers/buffers in the incubator.

According to some embodiments of the invention, the providing pressure stabilizers/buffers in the incubator comprises providing the pressure stabilizers before the rotational module.

According to some embodiments of the invention, the providing pressure stabilizers/buffers in the incubator comprises providing the pressure stabilizers after the rotational module.

According to an aspect of some embodiments of the present invention there is provided a method of incubating fetuses in an incubator, comprising:
a. flowing mixed gases at a determined concentration into the incubator;
b. flowing the mixed gases at a determined hyperbaric level;
c. maintaining the determined concentration and the determined hyperbaric level substantially constant;
d. buffering the mixed gases to maintain the determined concentration and the determined hyperbaric level substantially constant.

According to some embodiments of the invention, the culturing is effected using the fetal incubation system of any one of claims 42-75.

According to some embodiments of the invention, the method comprises manipulating the embryo prior to, during or following the culturing.

According to some embodiments of the invention, the manipulating comprises introducing into the embryo a gene of interest.

According to some embodiments of the invention, the manipulating comprises microinjecting cells into the embryo to thereby obtain a chimeric embryo.

According to some embodiments of the invention, the cells are stem cells.

According to some embodiments of the invention, the cells are xenogeneic cells.

According to some embodiments of the invention, the cells are human cells.

According to some embodiments of the invention, the manipulating comprises introducing into the embryo a drug of interest.

According to some embodiments of the invention, the method comprising determining an effect of the manipulating on development of the embryo.

According to some embodiments of the invention, the method comprising isolating a cell, tissue or organ from the embryo following the culturing.

According to some embodiments of the invention, the cells are selected from the group consisting of stem cells, blood cells, liver cells, pancreatic beta cells, lung epithelial cells, endothelial cells and glial cells.

According to some embodiments of the invention, the embryo might be a synthetic embryo formed by co-aggregating different types of pluripotent, trophoblast and primitive endoderm stem cells.

According to an aspect of some embodiments of the present invention there is provided a method of ex-utero culturing a mouse embryo, the method comprising culturing mouse embryo at an implanting blastocyst stage according to the method disclosed herein so as to obtain said embryo of said posterior neuropore closure to hind limb formation stage; and
culturing said embryo of said posterior neuropore closure to hind limb formation stage in a dynamic culture under a fourth set of conditions that allow development of said embryo to a indented anterior footplate stage, wherein said forth set of conditions comprise a hyperbaric pressure of more than 5 and less than 10.2 pounds per square inch (psi); an atmosphere comprising 30-95% oxygen; and said second medium comprising said at least 3 mg/ml glucose.

According to some embodiments of the invention, the culturing is from embryonic day (E)4.5 to E13.5.

According to some embodiments of the invention, the base medium further comprises sodium pyruvate.

According to some embodiments of the invention, the base medium comprises at least 1 mM.

According to an aspect of some embodiments of the present invention there is provided a method of ex-utero culturing a mouse embryo, the method comprising culturing a mouse embryo at a posterior neuropore closure to hind limb formation stage in a dynamic culture under conditions that allow development of said embryo to an indented anterior footplate stage, wherein said conditions comprise hyperbaric pressure of more than 5 and less than 10.2 pounds per square inch (psi); an atmosphere comprising 30-95% oxygen; and a medium comprising at least 30% serum, wherein said serum comprises rat serum and human serum, and a base medium comprising at least 3 mg/ml glucose.

According to some embodiments of the invention, the culturing is from embryonic day (E)10.5 to E13.5.

According to an aspect of some embodiments of the present invention there is provided a method of ex-utero culturing a mouse embryo, the method comprising culturing a mouse embryo at a late gastrulation stage in a dynamic culture under conditions that allow development of said embryo to a hind limb formation stage, wherein said conditions comprise a hyperbaric pressure of more than 5 and less than 10.2 pounds per square inch (psi); an atmosphere comprising increasing oxygen concentrations throughout said culturing starting from 5% up to 15-40%; and a medium comprising at least 30% serum, wherein said serum comprises rat serum and human serum, and a base medium comprising at least 1 mg/ml glucose up to an early somite stage and at least 3 mg/ml glucose when said embryo reaches said early somite stage.

According to some embodiments of the invention, the culturing is from embryonic day (E)7.5 to E11-11.5.

According to some embodiments of the invention, the increasing is effected every 20-28 hours of said culturing.

According to an aspect of some embodiments of the present invention there is provided a method of ex-utero culturing a mouse embryo, the method comprising culturing a mouse embryo at a post implantation pre gastrulation to early gastrulation stage in a static culture under conditions that allow development of said embryo to an early somite stage, wherein said conditions comprise an atmosphere comprising 15-40% oxygen; and a medium comprising at least 30% serum, wherein said serum comprises rat serum and human serum, and a base medium comprising at least 1 mg/ml glucose.

According to some embodiments of the invention, the culturing is from embryonic day (E)5.5-6.5 to E8.5.

According to an aspect of some embodiments of the present invention there is provided a method of ex-utero culturing a mouse embryo, the method comprising culturing a mouse embryo at an implanting blastocyst stage in a static culture under conditions that allow development of said embryo to a post implantation pre gastrulation stage, wherein said conditions comprise an atmosphere comprising 15-40% oxygen; a medium comprising 15-75% serum; and at least one of the following:
(i) an incision in said implanting blastocyst to release fluid and tension from within said blastocyst cavity is made prior to said culturing;
(ii) said serum is provided in said medium in increasing concentrations throughout said culturing; and/or
(iii) said serum comprises a human serum for at least part of said culturing.

According to some embodiments of the invention, the serum comprises serum replacement.

According to some embodiments of the invention, the 15-75% serum comprises 20-40% serum.

According to some embodiments of the invention, the increasing serum concentrations is effected every 16-52 hours of said culturing.

According to some embodiments of the invention, the serum comprises rat and/or bovine serum.

According to some embodiments of the invention, the medium comprises a base medium comprising Insulin-Transferrin-Selenium-Ethanolamine (ITS-X), progesterone, 3,3′,5-Triiodo-L-thyronine (T3) and optionally sodium lactate.

According to some embodiments of the invention, the base medium further comprises N2 and B27.

According to some embodiments of the invention, the conditions comprise N2 and B27 in said base medium following 1-2 days of said culturing.

According to some embodiments of the invention, the 15-75% serum comprises 20-30% serum.

According to some embodiments of the invention, the medium comprises a base medium comprising at least 1 mg/ml glucose.

According to some embodiments of the invention, the culturing is from embryonic day (E)4.5 to E5.5.

According to an aspect of some embodiments of the present invention there is provided a method of ex-utero culturing a mouse embryo, the method comprising:
a. culturing a mouse embryo at a post implantation pre gastrulation to early gastrulation stage in a static culture under a first set of conditions that allow development of said embryo to a late gastrulation to early somite stage, wherein said first set of conditions comprise an atmosphere comprising 15-40% oxygen; and a first medium comprising at least 30% serum, wherein said serum comprises rat serum and human serum, and a base medium comprising at least 1 mg/ml glucose, so as to obtain an embryo of a late gastrulation to early somite stage; and
b. culturing said embryo of said late gastrulation to early somite stage in a dynamic culture under a second set of conditions that allow development of said embryo to a posterior neuropore closure to hind limb formation stage, wherein said second set of conditions comprise an atmosphere comprising 15-40% oxygen; and a second medium comprising at least 30% serum, wherein said serum comprises rat serum and human serum, and a base medium comprising at least 3 mg/ml glucose; and wherein said second set of conditions comprise a hyperbaric pressure of more than 5 and less than 10.2 pounds per square inch (psi) starting the latest when said embryo reaches said early somite stage.

According to some embodiments of the invention, the medium further comprises knockout serum replacement (KSR) in addition to said rat serum and said human serum.

According to some embodiments of the invention, the KSR partially replaces one of either said human serum, said rat serum or partially replaces a quantity of both.

According to some embodiments of the invention, the culturing is from embryonic day (E)5.5-6.5 to E11-11.5.

According to some embodiments of the invention, the culturing is from embryonic day (E)5.5-6.5 to E10.5.

According to an aspect of some embodiments of the present invention there is provided a method of ex-utero culturing a mouse embryo, the method comprising culturing mouse embryo at a post implantation pre gastrulation to early gastrulation stage according to the method as disclosed herein so as to obtain said embryo of said posterior neuropore closure to hind limb formation stage; and
culturing said embryo of said posterior neuropore closure to hind limb formation stage in a dynamic culture under a third set of conditions that allow development of said embryo to an indented anterior footplate stage, wherein said third set of conditions comprise a hyperbaric pressure of more than 5 and less than 10.2 pounds per square inch (psi); an atmosphere comprising 30-95% oxygen; and said second medium.

According to some embodiments of the invention, the culturing is from embryonic day (E)5.5-6.5 to E13.5.

According to an aspect of some embodiments of the present invention there is provided a method of ex-utero culturing a mouse embryo, the method comprising:
a. culturing a mouse embryo at an implanting blastocyst stage according to the method as disclosed herein so as to obtain said embryo of said post implantation pre gastrulation stage; and
b. culturing said embryo of said post implantation pre gastrulation stage under a second set of conditions that allow development of said embryo to a late gastrulation to early somite stage, wherein said second set of conditions comprise an atmosphere comprising 15-40% oxygen; and a second medium comprising at least 30% serum, wherein said serum comprises rat serum and human serum, and a base medium comprising at least 1 mg/ml glucose.

According to some embodiments of the invention, the medium further comprises knockout serum replacement (KSR) in addition to said rat serum and said human serum.

According to some embodiments of the invention, the KSR partially replaces one of either said human serum, said rat serum or partially replaces a quantity of both.

According to some embodiments of the invention, the (b) is effected in a static culture.

According to some embodiments of the invention, the (b) is effected in a static culture followed by a dynamic culture.

According to some embodiments of the invention, the (b) is effected in a dynamic culture.

According to some embodiments of the invention, the second set of conditions of said dynamic culture comprises a hyperbaric pressure of more than 5 and less than 10.2 pounds per square inch (psi).

According to some embodiments of the invention, the culturing is from embryonic day (E)4.5 to E7.5.

According to some embodiments of the invention, the culturing is from embryonic day (E)4.5 to E8.5.

According to an aspect of some embodiments of the present invention there is provided a method of ex-utero culturing a mouse embryo, the method comprising culturing mouse embryo at an implanting blastocyst stage according to the method of any one of claims 14-14.04 and 17 so as to obtain said embryo of said early somite stage; and
culturing said embryo of said late gastrulation to early somite stage in a dynamic culture under a third set of conditions that allow development of said embryo to a posterior neuropore closure to hind limb formation stage, wherein said third set of conditions comprise a hyperbaric pressure of more than 5 and less than 10.2 pounds per square inch (psi); an atmosphere comprising 15-40% oxygen; and said second medium comprising at least 3 mg/ml glucose.

According to some embodiments of the invention, the culturing is from embryonic day (E)4.5 to E11-11.5.

According to some embodiments of the invention, the culturing is from embryonic day (E)4.5 to E10.5.

According to an aspect of some embodiments of the present invention there is provided a method of ex-utero culturing a mouse embryo, the method comprising culturing mouse embryo at an implanting blastocyst stage according to the method as disclosed herein so as to obtain said embryo of said posterior neuropore closure to hind limb formation stage; and
culturing said embryo of said posterior neuropore closure to hind limb formation stage in a dynamic culture under a fourth set of conditions that allow development of said embryo to a indented anterior footplate stage, wherein said forth set of conditions comprise a hyperbaric pressure of more than 5 and less than 10.2 pounds per square inch (psi); an atmosphere comprising 30-95% oxygen; and said second medium comprising said at least 3 mg/ml glucose.

According to some embodiments of the invention, the culturing is from embryonic day (E)4.5 to E13.5.

According to some embodiments of the invention, the said base medium further comprises sodium pyruvate.

According to some embodiments of the invention, the base medium comprises at least 1 mM sodium pyruvate.

According to an aspect of some embodiments of the present invention there is provided a method of ex-utero culturing a rabbit embryo, the method comprising culturing a rabbit embryo at a somitogenesis to early organogenesis stage in a dynamic culture under conditions that allow development of said embryo to a three cerebral vesicles stage, wherein said conditions comprise hyperbaric pressure of more than 5 and less than 10.2 pounds per square inch (psi); an atmosphere comprising 15-40% oxygen; and a medium comprising at least 30% serum, wherein said serum comprises rabbit serum and human serum.

According to some embodiments of the invention, the medium further comprises knockout serum replacement (KSR) in addition to said rabbit serum and said human serum.

According to some embodiments of the invention, the KSR partially replaces one of either said human serum, said rabbit serum or partially replaces a quantity of both.

According to some embodiments of the invention, the culturing is from gestation day (GD)9 to GD12.

According to an aspect of some embodiments of the present invention there is provided a method of ex-utero culturing a rabbit embryo, the method comprising culturing a rabbit embryo at a gastrulation stage in a dynamic culture under conditions that allow development of said embryo to an early organogenesis stage, wherein said conditions comprise an atmosphere comprising 15-40% oxygen; and a medium comprising at least 15% serum, wherein said serum comprises rabbit serum.

According to some embodiments of the invention, the culturing is from gestation day (GD)6 to GD9-10.

According to an aspect of some embodiments of the present invention there is provided a method of ex-utero culturing a rabbit embryo, the method comprising culturing a rabbit embryo at a blastocyst stage in a static culture under conditions that allow development of said embryo to a gastrulation stage, wherein said conditions comprise an atmosphere comprising 15-40% oxygen; a medium comprising 15-75% serum, wherein said serum comprises rabbit serum.

According to some embodiments of the invention, the culturing is from gestation day (GD)4 to GD6-7.

According to an aspect of some embodiments of the present invention there is provided a method of ex-utero culturing a rabbit embryo, the method comprising:
a. culturing a rabbit embryo at a blastocyst stage according to the method of any one of claims 19.13-19.15 so as to obtain said embryo of said gastrulation stage; and
b. culturing said embryo of said gastrulation stage under a second set of conditions that allow development of said embryo to a three cerebral vesicles stage, wherein said second set of conditions comprise a dynamic culture, an atmosphere comprising 15-40% oxygen; a medium comprising 15-75% serum.

According to some embodiments of the invention, the culturing is from gestation day (GD)4 to GD12.

According to some embodiments of the invention, the conditions comprise at least 30% serum, wherein said serum comprises rabbit serum and human serum, starting the latest when said embryo reaches a somitogenesis stage.

According to some embodiments of the invention, the conditions comprise hyperbaric pressure of more than 5 and less than 10.2 pounds per square inch (psi), starting the latest when said embryo reaches a somitogenesis stage.

According to some embodiments of the invention, the medium comprises a base medium comprising at least 1 mg/ml glucose.

According to some embodiments of the invention, the at least 30% serum comprises at least 50% serum.

According to some embodiments of the invention, the glucose is provided in said medium in increasing concentrations throughout said culturing.

According to some embodiments of the invention, the at least 1 mg/ml glucose comprises at least 3 mg/ml glucose.

According to some embodiments of the invention, the 15-40% oxygen comprises 19-23% oxygen.

According to some embodiments of the invention, the hyperbaric pressure is 6-7 psi.

According to an aspect of some embodiments of the present invention there is provided a fetal incubation system for at least one embryo, comprising:
a. a gas controller, configured for providing a plurality of gases to at least one incubator;
b. at least one incubator comprising a rotating module inside of said at least one incubator; rotating module comprising one or more vials comprising said at least one embryo; wherein said system comprises one or more buffers for said plurality of gases being provided to said rotating module inside of said at least one incubator.

According to some embodiments of the invention, the one of said one or more buffers is a gas mixing box for mixing said plurality of gases before being provided to said at least one incubator.

According to some embodiments of the invention, the gas controller comprises one or more specific gas controllers for individually control flow of specific one or more gases.

According to some embodiments of the invention, the gas controller comprises one or more electric valves for allowing flowing of said specific one or more gases.

According to some embodiments of the invention, the gas controller comprises a vacuum pump for extracting mixed gases from said gas mixing box.

According to some embodiments of the invention, the gas controller comprises a pressure pump in connection with said vacuum pump for providing said mixed gases to said system at hyperbaric pressures.

According to some embodiments of the invention, the pressure pump provide gases at pressures of from about 0.1 psi to about 20 psi.

According to some embodiments of the invention, the gas mixing box comprises one or more gas sensors.

According to some embodiments of the invention, the one or more specific gas controllers control activation and deactivation of said one or more electric valves according to said information received by said one or more gas sensors.

According to some embodiments of the invention, further comprising a humidifier, which also functions as one of said buffers for said plurality of gases.

According to some embodiments of the invention, the rotational module comprises a rotational drum comprising said one or more vials; said rotational drum connected to said humidifier.

According to some embodiments of the invention, the incubator comprises an outlet bottle for gases.

According to some embodiments of the invention, the outlet bottle for gases functions as one of said buffers for said plurality of gases.

According to some embodiments of the invention, the one or more buffers are configured to maintain a determined concentration of said plurality of gases and a determined hyperbaric level substantially constant.

According to an aspect of some embodiments of the present invention there is provided a method of incubating fetuses in an incubator, comprising:
a. flowing mixed gases at a determined concentration into a rotational module located inside said incubator;
b. flowing said mixed gases at a determined hyperbaric level;
c. maintaining said determined concentration and said determined hyperbaric level substantially constant.

According to some embodiments of the invention, the achieving said mixed gases at said determined concentration, comprises:
a. setting desired concentrations of each individual gas of said mixed gases;
b. flowing said individual gases into a gas mixing box;
c. sensing when each of said individual gases reaches said desired concentration;
d. mixing said gases inside said gas mixing box.

According to some embodiments of the invention, the achieving said determined hyperbaric level, comprises:
a. setting a desired hyperbaric level;
b. allowing access of said mixed gases to a pressure pump until said hyperbaric level is reached.

According to some embodiments of the invention, the maintaining comprises providing a plurality of sensors to said gas mixing box for monitoring said concentrations of said each individual gas.

According to some embodiments of the invention, the maintaining comprises providing pressure stabilizers/buffers in said incubator.

According to some embodiments of the invention, the providing pressure stabilizers/buffers in said incubator comprises providing said pressure stabilizers before said rotational module.

According to some embodiments of the invention, the providing pressure stabilizers/buffers in said incubator comprises providing said pressure stabilizers after said rotational module.

According to some embodiments of the invention, the culturing is effected using the fetal incubation system as disclosed herein.

According to some embodiments of the invention, the method comprises manipulating said embryo prior to, during or following said culturing.

According to some embodiments of the invention, the manipulating comprises introducing into said embryo a polynucleotide of interest.

According to some embodiments of the invention, the manipulating comprises introducing into said embryo a genome editing or RNA silencing agent.

According to some embodiments of the invention, the manipulating comprises microinjecting cells into said embryo to thereby obtain a chimeric embryo.

According to some embodiments of the invention, the manipulating comprises introducing into said embryo a drug of interest.

According to some embodiments of the invention, the method comprising isolating a cell, tissue or organ from said embryo following said culturing.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-K demonstrate an ex utero culture system for growing mouse late-gastrulating embryos until advanced organogenesis. FIG. 1A shows a schematic representation of the E7.5 embryo ex utero culture platform. FIG. 1C demonstrate developmental defects in embryos cultured with a deficit of glucose (n=24 embryos), low atmospheric pressure (n=24 embryos), or in constant 21% oxygen concentration (n=26 embryos). FIG. 1D shows bright-field images of embryos developing in utero from E7.5 to E11.5 and equivalent embryos cultured ex utero under the conditions shown in FIG. 1A. FIG. 1E is a graph demonstrating the percentage of developmentally normal embryos per culture day. “n”—total number of embryos; “x”—number of experiments. FIG. 1F is a graph demonstrating quantification of embryonic length for in utero and cultured embryos. Dots represent individual embryos; n(in utero)=13, 19, 15, 38; n(ex utero)=32, 15, 43, 41; ns—not significant, according to Mann-Whitney test. FIG. 1G shows a representative image of an embryo grown in bottle. FIGS. 1H-J show Sox2, Sox9 and Sox17 whole-mount immunofluorescence of embryos developed ex utero from E7.5. Insets are enlargements of the dashed boxes. Images represent a minimum of three biological replicates. FIG. 1K shows GFP fluorescence and bright field images of in utero E10.5 and ex utero +Day 3 IG-DMR-GFP reporter embryos. n=7 in utero; n=7 ex utero. All data are mean±s.e.m. Am—amnion; Al—allantois; Ch—chorion; D—diencephalon; Epi—epiblast; EPC—ectoplacental cone; Fg—foregut pocket; FL—forelimb bud; H—heart; HL—hindlimb bud; LV—lens vesicle; Md—mandibular arch; Ms—mesencephalon; Mt—metencephalon; Mx—maxillary arch; My—myelencephalon; NF—neural folds; OlP—olfactory placode; OP—optic pit; Otp—otic pit; PN—posterior neuropore; Pro—prosencephalon; Rho—rhombencephalon; S—somites; Sc—spinal cord; T—telencephalon; TB—tail bud; UC—umbilical cord; YS—yolk sac; YSV—yolk sac vessel. Scale bars, 500 μm.

FIGS. 2A-F demonstrate ex utero culture system for recapitulating mouse gastrulation. FIG. 2A shows a schematic representation of the static culture protocol for growing gastrulating embryos until somitogenesis. FIG. 2B shows bright field image of embryos developing ex utero from E6.5 until E8.5; percentage of properly developed embryos is indicated (bottom right). n=421 at +Day 1, n=399 at +Day 2. FIG. 2C shows live imaging snapshots of mouse embryo development from early gastrulation to somitogenesis (E6.5-E8.5, 2 hours intervals). n=6 embryos. FIG. 2D demonstrate cultured embryos immunostained for Sox2 (magenta) and Brachyury (T, red). Image are representative of a minimum of 3 biological replicates. FIG. 2E demonstrates scRNA-seq analysis of in utero E8.5 (purple dots) vs. E6.5 +Day 2 ex utero developing embryos (green dots). UMAP plot displaying individual cells (n=6358 ex utero +Day 2; n=4349 in utero E8.5). FIG. 2F demonstrates cell lineage annotation of clusters based on marker genes of the major cell types identified in E8.5 mouse embryos¹⁹. Points are colored according to their assigned cell cluster. Am—amnion; AB—allantoic bud; Al—allantois; Ch—chorion; Epi—epiblast; EPC—ectoplacental cone; ExE—extraembryonic ectoderm; Fg—foregut pocket; H—heart; NF—neural folds; PS—primitive streak; S—somites; VE—visceral endoderm; YS—yolk sac. Scale bars, 100 μm.

FIGS. 3A-H demonstrate ex utero culture system for growing mouse pre-gastrulation embryos until advanced organogenesis. FIG. 3A shows a schematic representation of the protocol for culturing mouse embryos from pre-gastrulation to organogenesis. FIG. 3B shows bright field images of embryos growing during five days ex utero from E6.5 to the 44-somites stage. Embryos cultured beyond day two are shown without the yolk sac. The variation in somite number is indicated. n is specified in FIG. 3E. Scale bars, 500 μm. FIG. 3C shows iDISCO immunostainings of early-gastrulating embryos grown ex utero during 3, 4 and 5 days. Images are representative of a minimum of 3 biological replicates. FIG. 3D shows bright field and immunostaining images of pre-gastrulating (E5.5) embryos cultured for six days until the 42 somites stage. Lefty1 and Oct4 immunostaining on a section of an E5.5 embryo (upper panel); n=3 embryos. Maximum intensity projection of an embryo fixed at culture day 6 and stained for Gata4, MHC-II and Sox2 (lower panel); n=3 embryos. Scale bars represent 50 μm (E5.5) and 500 μm (all others). FIG. 3E-F shows graphs demonstrating percentage of normal embryos in cultures started at E6.5 (FIG. 3E) and E5.5 (FIG. 3F). “n”—total number of embryos; “x”-number of experiments; data represent mean±s.e.m. FIG. 3G shows comparative scRNA-seq analysis of E6.5 +Day 4 ex utero embryos (green dots) and equivalent E10.5 embryos developing in utero (purple dots). UMAP plot depicting all cells considered in the analysis (n=39374 ex utero; n=24107 in utero). FIG. 3H shows cell lineage annotation of clusters based on the expression of marker genes described in the mouse organogenesis cell atlas²¹. Points are colored according to their assigned cell cluster.

FIGS. 4A-O demonstrate functional outcomes of perturbations introduced into the ex utero whole-embryo culture platform. FIG. 4A shows a schematic representation of the ex utero electroporation protocol at E8.5. FIG. 4B shows immunofluorescence of electroporated embryos stained for GFP, Sox2 and Tuj1. n=17, 15 and 11 embryos. FIG. 4C shows a schematic representation of the lentiviral transduction of E6.5 mouse embryos. FIG. 4D shows GFP, Sox2 and Gata4 immunostainings of E6.5 embryos transduced with GFP using lentivirus and grown ex utero for 1-5 days. n=15, 24, 19, 16 and 20 embryos. FIG. 4E shows a schematic representation of the generation of post-implantation chimeras by microinjection of primed EpiSCs, EpiLCs and E7.5 in vivo epiblast. A=anterior; D=distal; P=posterior. FIG. 4F is a graph demonstrating percentage of chimeric embryos (GFP⁺ or tdT⁺) following injection and ex utero culture. “exp”—number of experiments; “n”—number of embryos. FIG. 4G is a graph demonstrating quantification of GFP⁺ cells in chimeric embryos. Dots represent individual embryos; data are mean±s.e.m.; n(EpiSCs)=21, 3, 1, 1; n(EpiLCs)=22, 9; n(E7.5 Epiblast)=34, 15, 5, 7. *** p<0.0001; ** p=0.001; * p=0.0025 according to Mann-Whitney test. FIG. 4H shows EpiSC and EpiLC-chimeric embryos immunostained for GFP, Sox2 and Gata4, 1-2 days following injection. FIG. 4I shows immunostainings of embryos grafted with tdTomato⁺ E7.5 in vivo epiblast and cultured ex utero during 1-4 days. FIG. 4J shows a schematic representation of the protocol for generating human-mouse microglia chimeras. FIG. 4K shows bright field and fluorescence images of E7.5 embryos injected with GFP⁺ human microglia progenitors at day 0. FIG. 4L shows immunofluorescence of ex utero cultured human-mouse microglia chimeric embryos. n=11 and 8 embryos. FIG. 4M shows tdT⁺ embryos explanted at E7.5 and subjected to in toto live imaging of neural tube closure at E9.0. n=3. Scale bars represent 100 μm (FIG. 4M) and 500 μm (all others). FIG. 4N shows tdT⁺ embryos explanted at E7.5 and subjected to in toto live imaging of neural tube closure at E9.0. Scale bars represent 100 μm. FIG. 4O shows embryos cultured ex utero since E7.5 and exposed to vehicle or 1 mM valproic acid from E8.5 to E9.5. n=6. White arrows indicate neural tube closure defects. Insets shows magnification of the dashed boxes. Scale bars represent 500 μm.

FIGS. 5A-M demonstrate an optimized gas and pressure regulating controller for roller culture incubators. FIG. 5A is a schematic representation of a fetal incubation system. FIG. 5B is an image of an exemplary fetal incubation system. FIG. 5C is a diagram depicting the configuration of the electronic module for gas and pressure regulation. O₂, N₂ and CO₂ enter into the system at a pressure of 0.5 psi and are mixed into by a centrifugal blower. Gases are then injected into a water bottle inside the incubator by a pump that allows control of the gas pressure (hyperbaric conditions). Lph=liters per hour. FIG. 5D is a schematic representation of an exemplary gas and pressure controller. FIG. 5E shows a perspective view of an exemplary gas and pressure controller configured to monitor and manipulate CO₂ and O₂ levels by providing CO₂ and/or N₂. FIG. 5F show a top view of the gas and pressure controller open and showing internal components. FIG. 5G is a front view of the gas and pressure controller. FIG. 5H is a schematic representation of an exemplary gas mixing box. FIG. 5I is an image of an exemplary gas mixing box. FIG. 5J is a schematic representation of an exemplary incubator. FIG. 5K is an image of the interior of the precision incubator system (B.T.C. Engineering) showing the direction of the gas flow (indicated by the white arrowheads). FIG. 5L is an image of day 3 (E10.5) embryos cultured in rotating bottles (yellow arrowheads). FIG. 5M is a flowchart of an exemplary method related to the fetal incubation system.

FIGS. 6A-C demonstrate establishment and optimization of a mouse embryo ex utero culture from late gastrulation (E7.5) until advanced organogenesis (E11). FIG. 6A shows E7.5 embryo dissection overview. FIG. 6B demonstrate percentage of normally developed embryos under different gas pressure, glucose or oxygen concentration. Blue numbers indicate the conditions yielding the highest efficiency of embryo survival. Values in parenthesis denote the number of embryos assessed per condition in every sampled time-point. Embryos dissected, fixed or moved to other conditions are subtracted from the total. Representative bright field images of embryos cultured under certain conditions are shown. FIG. 6C demonstrate efficiency of normal embryonic development evaluated in mice of different genetic backgrounds. Parental mouse lines are indicated on the left (female: male). Values in parenthesis show the number of embryos evaluated. EUCM—ex utero culture media; HPLM—human plasma-like media; KSR—knockout serum replacement; mg—milligrams; psi—pounds per square inch; PYS parietal yolk sac; RS—rat serum. Scale bars, 500 μm.

FIG. 7 demonstrate that spatio-temporal expression patterns of ectoderm- and mesoderm-related lineage markers are recapitulated in the ex utero cultured embryos. Maximum intensity projections of embryos developed in utero and ex utero, fixed and immunostained for Sox2, Otx2, Tuj1, Pax6, Sox9, Brachyury, Cdx2 and MHC-II (Myosin Heavy Chain-II) at the indicated stages. Blue, DAPI. Image are representative of a minimum of 3 biological replicates. Scale bars, 100 μm for E7.5, 200 μm for E8.5/9.5, and 500 μm for E10.5/E11.

FIG. 8 demonstrate that in vivo spatio-temporal expression patterns of endoderm-related lineage markers are recapitulated in cultured embryos. Maximum intensity projections of embryos developed in utero and ex utero, fixed and immunostained for Sox17, Foxa2 and Gata4 at the indicated stages. Blue—DAPI. For Sox17, insets are enlargements of the dashed boxes. Representative immunohistochemistry mid-section (sagittal plane) images are shown for Foxa2 and Gata4 at the last time-point (far-right panels). Images represent a minimum of 3 biological replicates. Scale bars=100 μm for E7.5, 200 μm for E8.5/9.5, and 500 μm for E10.5/E11.

FIGS. 9A-B demonstrate ex utero culture of GFP-reporter transgenic embryos. FIG. 9A shows bright field and GFP fluorescence images of ex utero embryos in culture at the specified times expressing the GFP reporter following activation by Wnt1-Cre and Isl1-Cre lineage-specific reporter alleles. n=7 and 10 embryos, respectively. Embryos dissected out of the yolk sac at +Day 4 are shown in the far-right panel. FIG. 9B shows representative confocal images of in utero E11.5 and ex utero +Day 4 transgenic mouse embryos expressing GFP following activation by Wnt1-Cre and Isl1-Cre lineage-specific reporter alleles. Scale bars, 1 mm.

FIGS. 10A-O demonstrate the process of devising a platform for culturing mouse embryos from the onset of gastrulation until advanced organogenesis. Shown are schematic representation of the different protocol indicating the percentage of E6.5 embryos developed properly per day in each condition. The media composition, static or roller culture, and oxygen concentrations are specified for each protocol. Values in parenthesis denote the number of embryos evaluated per condition. Embryos dissected, fixed or moved to other conditions are subtracted from the total. Representative bright field images of embryos cultured under certain conditions are shown on the right side of the respective protocol. Numbers in blue indicate the protocol yielding the highest efficiency of embryo survival that was subsequently used throughout the study. Scale bars, 500 μm. EUCM—ex utero culture media; HBS—human adult blood serum; PSI—pounds per square inch; RS—rat serum.

FIGS. 11A-C demonstrate that embryos grown ex utero since early gastrulation recapitulate the spatio-temporal expression profiles of lineage markers seen in utero. Shown are maximum intensity projections of embryos developed in utero and ex utero, fixed and immunostained for eleven specific markers at the indicated time-points. Blue-DAPI. Images are representatives of a minimum of 3 biological replicates. Scale bars, 50 μm for E6.5, 100 μm for +Day 1, 200 μm for +Day 2/3, 500 μm for +Day 4/5.

FIGS. 12A-H demonstrate single cell transcriptomic analysis of ex utero +Day 2/Day 4 cultured embryos compared to in utero E8.5/E10.5 embryos. FIG. 12A shows a schematic representation of the embryo culture protocol and sequenced time-points. Pre-gastrulating (E6.5) embryos grown ex utero were processed for 10× genomics single cell RNA-sequencing following 2 and 4 days of culture. FIG. 12B show violin plots indicating the number of UMIs and genes obtained per condition at each time-point (E8.5, median of 9787 UMIs and 2989 genes detected per cell; E10.5, median of 4795 UMIs and 1789 genes were detected per cell). FIG. 12C-D show lineage annotation at culture day +2 (FIG. 12C) and +4 (FIG. 12D). Dot-plot illustrating the area under the curve (AUC) enrichment value of overlapping cells across clusters and tissue lineages. Circle size denotes the magnitude of enrichment. Colors indicate p-value (calculated based on AUC). FIG. 12E-F show UMAP-based plots illustrating the normalized AUC assigned value of all individual cells for each lineage at culture day +2 (FIG. 12E) and +4 (FIG. 12F). FIG. 12G shows correlation of gene expression of the top 2000 most variable genes per cluster between in utero E10.5 and ex utero +Day 4 embryos. Differentially expressed genes are named and shown as red dots. Clusters with the highest number of variable genes (range of 2-8 genes only per cluster) are encased in a red box. FIG. 12H shows pie-charts depicting the proportional abundance of each cell cluster in both in ex utero and utero developed embryos at +Day 4/E10.5. Asterisks denote clusters with statistically significant differences between the two groups. p-values: Cluster 7=0.004, Cluster 8=0.009, Cluster 15=0.001.

FIGS. 13A-E demonstrate morphological and size changes in embryos developing ex utero from pre-gastrulation to the hindlimb formation stage. FIG. 13A shows a proportional increase in size of ex utero embryos grown from the onset of gastrulation (E6.5) to the 44 somites stage. Representative bright field images of embryos cultured for 5 days, are shown at each specific stage. Embryos without yolk sac are shown from day 3 to 5. n>119. FIG. 13B is a schematic diagram depicting the embryonic axis measured at each stage (length of the antero-posterior axis for E6.5 to E8.5 and crown-rump length for later stages). FIG. 13C is a graph summing the measurements of embryonic length (μm) at the indicated time-points. Dots represent individual embryos; n(in utero)=72, 25, 13, 19, 15, 38; n(ex utero)=68, 29, 8, 19, 24; ns—not significant according to Mann-Whitney test. FIG. 13D shows bright field images of an E5.5 embryo grown ex utero during 6 days until the 42-somites stage. Embryos cultured since E5.5 exhibit a mild developmental delay of about 2-4 pairs of somites when compared to in utero, yet, overall morphological development seemed to occur correctly. FIG. 13E shows a representative increase in size of embryos cultured from E5.5 to the hindlimb stage (6 days of culture). Embryos dissected at the beginning and end of culture are shown. n=minimum of 5 embryos. Scale bars, 100 μm for E5.5 in FIG. 13D; and 500 μm for all others. A-P—antero-posterior axis.

FIGS. 14A-C demonstrate that ex utero culturing in a EUCM media supplemented with human adult blood serum (HBS) instead of human umbilical cord serum (HCS) supports embryo development from early/late gastrulation until the hindlimb stage E11. FIG. 14A-B show bright field microscopy images of mouse embryos grown ex utero from E7.5 (FIG. 14A) or E6.5 (FIG. 14B), in which freshly isolated in-house prepared human umbilical cord serum (HCS) was replaced with in house prepared and freshly isolated adult human blood serum (HBS). FIG. 14C shows graphs demonstrating percentage of normal and defective embryos in cultures started at E7.5 and E6.5. “exp”—number of experiments conducted; “n”—total number of cultured embryos. Data represent mean±s.e.m. Scale bars, 500 μm. FIGS. 15A-L demonstrate analysis of ex utero electroporation, lentiviral transduction and mouse post-implantation chimeric embryos. FIGS. 15A-B show graphs demonstrating percentage of developmentally normal (FIG. 15A) and GFP-expressing embryos (FIG. 15B) at 1-3 days following electroporation. FIG. 15C is a graph demonstrating quantification of GFP⁺ cells in electroporated embryos at the indicated times. Dots represent individual embryos. FIG. 15D-E are graphs demonstrating percentage of normally developed (FIG. 15D) and GFP⁺ embryos (FIG. 15E) following lentiviral transduction. “x”—number of experiments conducted; “n”—total number of cultured embryos assessed. Data represent mean±s.e.m. FIG. 15F shows representative qPCR demonstrating the relative expression levels of mouse naïve and primed markers in V6.5 mouse EpiSCs and formative EpiLCs, normalized to isogenic naïve 2i/Lif ESCs. n=3. FIG. 15G shows overlap in the transcriptional signature of differentially expressed genes measured by bulk RNA-seq in EpiSCs and ESCs used herein, compared to previously published datasets by Wu et al²⁶. n=2. FIG. 15H demonstrate the generation of intraspecies chimeras using isogenic naïve ESCs. A schematic representation of the protocol is shown in the upper right panel; bright field and fluorescent GFP images of chimeric embryos generated with naïve ESCs are shown in the bottom left panel; and GFP, Sox2 and Gata4 immunofluorescence are shown in the right panels. FIG. 15I shows whole-mount immunostaining of GFP⁺ cells detected in embryos injected with mouse EpiSCs or EpiLCs at E7.5, cultured ex utero 1-4 days and stained for GFP, Sox2 and Gata4. Insets are enlargements of the dashed boxes. n>8 embryos. FIG. 15J shows immunostaining of +Day 1 cultured embryos injected with EpiSCs and EpiLCs in the anterior or distal epiblast. Images represent a minimum of 3 biological replicates. FIG. 15K shows representative confocal images of mouse post-implantation chimeras generated by tdT+E7.5 in vivo epiblast orthotopic transplantation followed by ex-utero culture for 1-4 days, stained for tdTomato, Gata4/Sox9 and Sox2/Tuj1. n>10 embryos. FIG. 15L shows embryos cultured ex utero since E7.5 and exposed to vehicle or 1 mM valproic acid from E8.5 to E9.5. n=6. White arrows indicate neural tube closure defects. Insets shows magnification of the dashed boxes. Scale bars, 500 μm.

FIGS. 16A-F demonstrate generation of human-mouse microglia interspecies chimeric embryos. FIG. 16A shows a schematic representation of the protocol for differentiation of microglia progenitors from humans ESCs as described in Wilgenburg et.al.²⁸ FIG. 16B shows flow cytometry dot-plots to validate the identity of obtained microglia cells by co-expression of the CD34⁺ and CD43⁺ microglia progenitor cell markers. n=3 independent experiments. FIG. 16C shows whole-mount immunostaining images of a human microglia chimeric mouse embryo stained for GFP (identifying human cells) and Tuj1. FIG. 16D is a graph demonstrating quantification of GFP⁺ cells detected in human-mouse microglia chimeric embryos (excluding GFP⁺ cells found in the yolk sac). Dots represent individual embryos; n=11 and 8 embryos. FIG. 16E shows immunostaining for GFP and human TMEM119 in chimeric embryos. n=3. FIG. 16F shows representative GFP immunofluorescence of a human microglia chimeric embryonic yolk sac and yolk sac vessel with circulating human GFP⁺ cells. n=3. Scale bars represent 50 μm (FIG. 16E) and 500 μm (all others).

FIGS. 17A-B demonstrate an ex utero culture system for growing mouse zygote embryos until gastrulation. FIG. 17A shows a schematic representation of the protocol for culturing mouse embryos from the 1-cell stage (day 0) until advanced gastrulation E7.5. Bright field images are shown at the indicated time-points. Percentage of properly developed embryos is shown for day 9 and 10. FIG. 17B shows whole-mount immunostaining for Oct4 (magenta), Brachyury (red) and Gata4 (green) on a zygote grown ex utero until the E7.5 stage. Scale bar, 100 μm.

FIGS. 18A-B demonstrate an ex utero culture system for growing mouse zygote embryos until somitogenesis. FIG. 18A shows a schematic representation of the protocol for culturing mouse zygotes until the early somite stage E8.5. FIG. 18B shows representative bright field images of embryos during each day of ex utero culture. Percentage of properly developed embryos is shown for day 9 to 11.

FIG. 19 demonstrate that somitogenesis stage embryos cultured ex-utero from the zygote stage express anterior and posterior lineage markers. Shown are maximum intensity projection images of the ventral and dorsal side of an embryo stained for Cdx2, Brachyury and Sox2 following 11 days of culture. Nuclei are counterstained with Dapi. Scale bar, 100 μm.

FIGS. 20A-B demonstrate an ex utero culture system for growing mouse since pre-gastrulation up to E13.5. FIG. 20A shows a schematic representation of the protocol for culturing mouse embryos from E6.5 until E13.5. FIG. 20B shows bright field images of the cultured embryos at the indicated time-points during the 7 days of culturing.

FIG. 21 demonstrates that addition of 1 mM sodium pyruvate to EUCM promotes forebrain growth and eliminates forebrain defects. Blue arrows indicate eye and forebrain region and size.

FIGS. 22A-C demonstrate an ex utero culture system for growing mouse zygote embryos to organogenesis. FIG. 22A shows a schematic representation of the protocol for culturing mouse embryos from the 1-cell stage (day 0) until E9.5. FIG. 22B shows bright field images at the indicated time-points during the 12 days of culturing. FIG. 22C shows whole-embryo immunostaining z-section images of a zygote developed in-vitro until E7.5 egg-cylinder, stained for Oct4 (magenta), Brachyury (red) and Gata4 (green). Nuclei were counterstained with DAPI (blue). White arrow indicates the most anterior site of Brachyury⁺ cells migration. Yellow arrow indicates the amnion. Fg, foregut pocket; H, heart; OP, optic pit; OtP, otic pit; S, somites; Sc, spinal cord; YS, yolk sac. Scale bars, 100 m; 500 m where indicated.

FIGS. 23A-D demonstrate no gastrulation or organogenesis following ex utero culturing mouse zygote embryos using the previously described media IVC1 and IVC2 (Bedzhov et al. Cell 2014 PMID: 24529478). FIG. 23A shows a schematic representation of the protocol described in Bedzhov et al. FIG. 23B shows phase contrast and whole-mount immunostaining for Cdx2 (magenta), Gata4 (red) and Oct4 (green). Nuclei were counterstained with DAPI (blue). The Figures shows only small distorted stage embryos that have not initiated gastrulation even at day 5 of the protocol. In fact, the images show an empty yolk sac in which the epiblast could not survive and thus have no embryo morphology. FIG. 23C shows a schematic representation of the protocol based on FIG. 22A using the IVC1 and IVC2 media. FIG. 23D is a representative phase contrast image demonstrating a distorted small embryos that does not show gastrulation or organogenesis at the end of the protocol.

FIGS. 24A-C demonstrate an ex utero culture system for growing mouse zygote embryos to organogenesis. FIG. 24A shows a schematic representation of the protocol for culturing mouse embryos from the 1-cell stage (day 0) until E9.5-10.5, using EUCM2/3/4 media instead of EIVC1 and EIVC2. FIG. 24B shows bright field images of the cultured embryos at the indicated time-points during the 13 days of culturing. FIG. 24C shows bright field images of the cultured embryos at the indicated time-points, wherein the culture protocol comprised EUCM/2/3/4 supplemented with NEAA, D-Glucose, ITS-X, β-Estradiol, Progesterone and N-acetyl L-Cysteine.

FIGS. 25A-C demonstrate an ex utero culture system for growing mouse zygote embryos to organogenesis applying laser mediated incision at the blastocyst stage. FIG. 25A shows a schematic representation of the protocol. FIG. 24B shows laser mediated incision (cut) using the Lykos Laser system by Hamilton thorne made following removal of the zona pellucida. FIG. 25C shows representative bright field images of the cultured embryos at day 10 and 11 of the protocol demonstrating the embryos correspond to developmental E10 and E11, respectively (i.e. the delay in progression was resolved by the protocol).

FIGS. 26A-B demonstrate generation of embryos from PSCs via combined use of tetraploid complementation and ex utero embryogenesis platforms. FIG. 26A shows a schematic representation of the protocol. Mouse zygotes obtained from mating of BDF1 mice, are subjected to electrofusion at the 2 cell stage as routinely practiced. WT V6.5 EGFP labeled mouse ESCs are microinjected at the 4n blastocyst stage. Instead of transferring these blastocysts back in utero, they are subjected to ex utero platforms described herein. FIG. 26B shows a representative image of an organized embryo obtained ex utero.

FIG. 27 shows schematic representation of protocols for generating a mutant embryo with restricted developmental potential.

FIG. 28 shows optimized settings for E6.5 embryo electroporation, having the best integration of target and showing high survival of embryos after electroporation.

FIG. 29 shows images of E7.5 embryos, 16 hours following electroporation with 2 μg/μL Atto-labelled tracrRNA (Alt-R Cas9 tracrRNA, ATTO 550, IDT, Cat. 1073190). The images show normal development together with high integration level based on the red fluorescent mark by the labelled tracrRNA.

FIG. 29 demonstrates normal development together with high integration level of Atto-labelled tracrRNA in E7.5 embryos, 16 hours after electroporation of E6.5 embryos.

FIGS. 30A-B demonstrate Lim1 knock-out via CRISPR via ex utero embryo electroporation. FIG. 30A shows a schematic presentation of the protocol. FIG. 30B shows images demonstrating defects in the head structure in embryos, 3 days following ex-utero electroporation and culture (E9.5).

FIGS. 31A-B demonstrate Lim1 knock-out via CRISPR via ex utero embryo lentiviral infection. FIG. 31A shows a schematic presentation of the protocol. FIG. 31B shows images demonstrating malformation of head of embryos (Box1 and Box2 show headless embryos), 3 days following ex-utero lentiviral infection and culture (E9.5).

FIGS. 32A-B demonstrate an ex utero culture system for growing rabbit Gestational Day (GD) 1 embryos to GD6. FIG. 32A shows a schematic representation of the protocol. FIG. 32B shows representative images of immunofluorescence staining of GD6 embryo demonstrating SOX2 epiblast (green) and CDX2 trophectoderm (red) on the outer part of the late blastocyst. Nuclei were counterstained with DAPI (blue).

FIGS. 33A-C demonstrate an ex utero culture system for growing rabbit GD6 embryos to GD9. FIG. 33A shows a schematic representation of the protocol. FIG. 33B shows representative images of ex-utero grown GD7-9 embryos. FIG. 33C shows that all known 8 stages of rabbit gastrulation were sequentially imaged ex utero using the protocol.

FIG. 34 demonstrates an ex utero culture system for growing rabbit GD9 embryos to GD12. Shown a schematic representation of the protocol and representative images of ex-utero grown GD9-12 embryos.

### DESCRIPTION OF SPECIFIC EMBODIMENTS OF THE INVENTION

The present invention, in some embodiments thereof, relates to methods and devices for ex-utero mouse embryonic development.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Development of a mammalian embryo takes place following implantation of the embryo in the uterus, which makes it relatively inaccessible for observation and manipulation. While mouse embryos are consistently cultured through pre- and peri-implantation development, establishing culture conditions sustaining proper long-term development of post-implanted mouse embryos outside the uterine environment remains challenging.

Whilst reducing specific embodiments of the present invention to practice, the present inventors have now developed a robust embryo culture system that faithfully recapitulates for the first time mouse in utero development from pre-gastrulation to advanced organogenesis stages and from Zygote stage up to day 13, enabling the application and monitoring of external and internal manipulations in mouse embryos over up to eight days of post-implantation development.

As is illustrated hereinunder and in the examples section, which follows, the present inventors show ex utero mouse embryo culture platforms, that enable appropriate development of embryos from the zygote stage [embryonic day (E) 0/0.5] or pre-gastrulation [E5.5] stage until the hind limb formation stage (E11/11.5) (Examples 1-3 and 5) and even further until the indented anterior footplate stage (E13.5). Specifically, late gastrulating embryos (E7.5) are grown in 3D rotating bottles settings, while extended culture from the zygote or pre-gastrulation stage (E5.5 or E6.5) requires a combination of novel static and rotating bottle culture protocols. Using histological, molecular, and single cell RNA-seq analyses, the present inventors demonstrate that the ex utero developed embryos recapitulate in utero development; and further show that this developed culture system is amenable to introducing a variety of embryonic perturbations and micro-manipulations that can be followed ex utero (See for example Examples 1-7).

Additionally, as illustrated hereinunder, the present inventors show a gas and pressure controller for the ex utero mouse embryo culture platforms, which enables a precise and stable control of the gas levels and pressure levels in each of the incubation chambers/bottles. In some embodiments, maintaining pressure is performed by the addition of one or more buffers in the pathway of the gases in the incubation system. An aspect of some embodiments of the invention relates to fetal incubation systems having rigorously monitored supply of gases. In some embodiments, the fetal incubation system are ex-utero external incubation system. In some embodiments, the fetal incubation system is connected to one or more independent gas sources, for example gas tanks comprising CO2, N2, H₂, water vapor or O₂. In some embodiments, the fetal incubation system comprises a controller configured for monitoring the gas and/or the mix of gases that are provided to the incubator. In some embodiments, the fetal incubation system further comprises a pressure pump for providing gases at hyperbaric levels. An aspect of some embodiments of the invention relates to providing and maintaining chosen pressure levels inside a fetal incubation system. In some embodiments, pressure is provided by means of a pressure pump, and pressure is preserved by means of one or more buffering stations along the path of the gas, optionally before entering the individual incubation chambers/bottles and after exiting them. In some embodiments, buffering station comprise one or more containers comprising a liquid into which the gases are delivered. In some embodiments, providing a mixture of gases comprises providing a mixture of gases into a rotating module containing one or more vials containing the embryos. In some embodiments, the rotation of the rotating module is independent of the provision of the mixed gases into the vials. In some embodiments, the rotating module is allocated inside the fetal incubation system.

Establishment of methods and systems for growing normal mouse embryos ex utero until advanced organogenesis may be further combined with e.g. genetic modification, chemical screens, tissue manipulation and microscopy methods and may constitute a powerful tool in basic research e.g. as a framework to investigate the emergence of cellular diversity, cell fate decisions and how tissues and organs emerge from a single totipotent cell; as well as a source of cells, tissue and organs for transplantation, generation of chimeric embryos, testing the effect of drugs on embryonic development etc.

Thus, according to an aspect of the present invention, there is provided a method of ex-utero culturing a mouse embryo, the method comprising culturing a mouse embryo at a posterior neuropore closure to hind limb formation stage in a dynamic culture under conditions that allow development of said embryo to an indented anterior footplate stage, wherein said conditions comprise hyperbaric pressure of more than 5 and less than 10.2 pounds per square inch (psi); an atmosphere comprising 30-95% oxygen; and a medium comprising at least 30% serum, wherein said serum comprises rat serum and human serum, and a base medium comprising at least 3 mg/ml glucose.

In some embodiments, optionally, the medium further comprises knockout serum replacement (KSR) in addition to the rat serum and the human serum.

In some embodiments, optionally, the KSR partially replaces one of either the human serum, the rat serum or partially replaces a quantity of both.

As used herein, the term “posterior neuropore closure” in the context of a mouse embryo refers to an embryo following the early somite stage and prior to the hind limb formation stage and is characterized by closure of the posterior neuropore. Typically, an embryo of a posterior neuropore closure is defined as Theiler stages TS15-TS16 (see Theiler stage definition in the emap database).

According to specific embodiments, the posterior neuropore closure stage refers to embryonic day (E) 10-10.5.

According to specific embodiments, the posterior neuropore closure stage refers to embryonic day (E) 10.5.

As used herein, the term “embryonic day (E)” in the context of a mouse embryo refers to an embryo having developmental characteristic of an in vivo (in-uterine tube or in utero, depending on the day) mouse embryo counterpart at the specified day following fertilization, wherein E0 is considered as the fertilized egg.

As used herein, the term “hind limb formation stage” in the context of a mouse embryo refers to an embryo following the neural tube closure stage and prior to the handplate stage and is characterized by the presence of paddle-shaped forelimbs and hindlimbs. Typically, an embryo of a hind limb formation stage is defined as Theiler stages TS17-TS18 (see Theiler stage definition in the emap database).

According to specific embodiments, hind limb formation stage refers to embryonic day (E) 11-11.5.

As used herein, the term “indented anterior footplate stage” in the context of a mouse embryo refers to an embryo following the anterior and posterior footplate stage and prior to Embryonic day 14.5 (TS22) and is characterized by the earliest sign of digits and 50-55 somites formed, 5 rows of whiskers and umbilical hernia are clearly apparent. Typically, an embryo of an indented anterior footplate stage is defined as Theiler stages TS21-TS22 (see Theiler stage definition in the emap database).

According to specific embodiments, indented anterior footplate stage refers to embryonic day (E) 13-13.5.

According to specific embodiments, indented anterior footplate stage refers to embryonic day (E) 13.5.

Embryonic stage and development may be assessed compared to an in vivo embryo counterpart at the same developmental stage by multiple ways including, but not limited to, morphology, length, weight, weight, expression of developmental marker genes (e.g. Oct4, Nanog, Sox2, Klf4, Cdx2, Gata4, Gata6, Brachyury, Otx2, Fgf5) using specific antibodies or primers, transcriptional profiling and the like, as further described hereinbelow and in the Examples section which follows which serve as an integral part of the specification.

Morphology assessment of embryonic development may be performed by previously established morphological features, such as described in e.g. Van Maele-Fabry, G., et al. Toxicol. Vitr. 4, 149-156 (1990); Van Maele-Fabry, G., et al. Int. J. Dev. Biol. 36, 161-167 (1992), the contents of which are fully incorporated herein by reference. Thus, for example, E7.5 may be characterized by a small allantois bud present at the base of the primitive streak, the amniotic folds fuse to form the amnion, the chorion is well developed and the anterior ectoderm begins to form the future neural groove. These events generate three cavities in the embryo: amniotic, exocoelomic and ectoplacental cavities. E10-10.05 may be characterized by 32-39 somites, tail bud and hindlimb buds, paddle-shaped forelimbs, posterior neuropore closed the fourth branchial arch is formed, visible division between telencephalon, diencephalon, mesencephalon, metencephalon and myelencephalon to form a five-vesicles brain. Further, embryos at this stage show a four-chambered heart, invaginating optic vesicle, olfactory plate formed, and the vessels of the yolk sac form a hierarchical network of large and small-caliber vessels with red blood cells circulating around the yolk sac and the body of the embryo. E11-11.5 may be characterized by tail bud clearly present, paddle-shaped forelimbs and hindlimbs, posterior neuropore closed, visible division between telencephalon, diencephalon, mesencephalon, metencephalon and myelencephalon to form a five-vesicles brain, four-chambered heart, invaginating optic vesicle, olfactory plate formed, vessels of the yolk sac form a hierarchical network of large and small-caliber vessels with red blood cells circulating around the yolk sac and the body of the embryo, presence of the fourth branchial arch, developed nasal pits, invagination and closure of the lens vesicle. e13-13.5 may be characterized by the earliest sign of digits, 50-55 somites, 5 rows of whiskers and umbilical hernia clearly apparent.

Developmental markers can be detected using immunological techniques well known in the art [described e.g. in Thomson J A et al., (1998). Science 282: 1145-7]. Examples include, but are not limited to, immunostaining, microscopy, flow cytometry, western blot, and enzymatic immunoassays. Other non-limiting methods include PCR analysis, RNA fluorescence in situ hybridization (FISH), northern blot, single cell RNA sequencing. Non-limiting Examples of specific markers for several developmental markers are provided in SEQ ID NOs: 3-22.

Culturing of an embryo starting from the posterior neuropore closure to hind limb formation stage embryo of some embodiments of the invention may be effected until reaching the indented anterior footplate stage or any developmental stage therein-between.

According to specific embodiments, culturing of an embryo starting from the posterior neuropore closure to hind limb formation stage is effected until reaching the indented anterior footplate stage.

According to specific embodiments, culturing of an embryo starting from the posterior neuropore closure to hind limb formation stage is continued also following reaching the indented anterior footplate stage.

According to specific embodiments, the culturing methods described herein are effected for at least at least 1 day, at least 2 days or at least 3 days, at least 4 days, at least 5 days, at least 6 days, at least 7 days.

According to specific embodiments, culturing of an embryo starting from the late gastrulation stage is effected for at least 1 day, at least 2 days or at least 3 days.

According to specific embodiments, culturing of an embryo starting from the posterior neuropore closure to hind limb formation stage is effected for 2-5, 2-4 or 2-3 days.

According to specific embodiments, culturing of an embryo starting from the posterior neuropore closure to hind limb formation stage is effected for about 3 days.

According to specific embodiments, culturing is from E10.5 to E13.5.

The posterior neuropore closure to hind limb formation stage embryo of some embodiments of the invention may be obtained by dissecting the embryo out from a uterus of a pregnant female mouse. Methods of obtaining live undamaged embryos are well known in the art for example in Kalaskar and Lauderdale (2014) Mouse Embryonic Development in a Serum-free Whole Embryo Culture System. Journal of Vis. Exp.

According to specific embodiments, the embryo is dissected into a dissection medium prior to the culturing. Such a dissection medium may comprise a base medium such as a synthetic tissue culture medium, e.g. DMEM supplemented with salts, nutrients, minerals, vitamins, amino acids, nucleic acids, and/or proteins such as cytokines, growth factors and/or hormones. According to specific embodiments, the dissection medium comprises glucose (e.g. 1 mg/ml). According to specific embodiments, the dissection medium comprises serum (e.g. 10% fetal bovine serum). According to specific embodiments, the dissection medium is equilibrated at 37° C. for at least half an hour prior to use.

According to specific embodiments, the method further comprises opening the embryonic (visceral) yolk sac, of the embryo to allow exposure of the embryo directly to oxygen and medium. Such an opening may be effected by completely taking the embryos out of the yolk sac and amnion, carefully avoiding rupture of any major yolk sac blood vessels, but keeping the yolk sac and umbilical cord attached to the embryo.

According to specific embodiments, opening of the yolk sac is effected when the embryo reaches at least the posterior neuropore closure stage.

According to specific embodiments, opening of the yolk sac is effected prior to the anterior and posterior footplate stage (Theiler stages TS19-TS20, about E12.5).

According to specific embodiments, opening of the yolk sac is effected prior to the indented anterior footplate stage.

According to specific embodiments, opening of the yolk sac is effected between E10.5-E13, between E11-13 or between E11-12.

According to other specific embodiment, the posterior neuropore closure to hind limb formation stage embryo is obtained from a previously cultured embryo. The present inventors have developed novel methods of culturing an embryo from the implanting blastocyst stage until at least the hind limb formation stage (see e.g. Examples 1, 3 and 5 of the Examples section which follows).

Thus, according to an aspect of the present invention, there is provided a method of ex utero culturing a mouse embryo, the method comprising culturing a mouse embryo at a late gastrulation stage in a dynamic culture under conditions that allow development of said embryo to a hind limb formation stage, wherein said conditions comprise a hyperbaric pressure of more than 5 and less than 10.2 pounds per square inch (psi); an atmosphere comprising increasing oxygen concentrations throughout said culturing starting from 5% up to 15-40%; and a medium comprising at least 30% serum, wherein said serum comprises rat serum and human serum, and a base medium comprising at least 1 mg/ml glucose up to an early somite stage and at least 3 mg/ml glucose when said embryo reaches said early somite stage.

In some embodiments, optionally, the medium further comprises knockout serum replacement (KSR) in addition to the rat serum and the human serum.

In some embodiments, optionally, the KSR partially replaces one of either the human serum, the rat serum or partially replaces a quantity of both.

As used herein, the term “late gastrulation stage” in the context of a mouse embryo refers to an embryo following the early gastrulation stage and prior to the early somite stage and is characterized by an egg cylinder-shaped embryo with differentiated definitive endoderm, mesoderm and ectoderm layers. Typically, an embryo of a late gastrulation stage is defined as Theiler stages TS10-TS11 (see Theiler stage definition in the emap database).

According to specific embodiments, the late gastrulation stage refers to embryonic day (E) 7-8.

According to specific embodiments, the late gastrulation stage refers to embryonic day (E) 7.5.

Culturing of an embryo starting from the late gastrulation stage embryo of some embodiments of the invention may be effected until reaching the hind limb formation stage or any developmental stage therein-between.

According to specific embodiments, culturing of an embryo starting from the late gastrulation stage is effected until reaching the hind limb formation stage.

According to specific embodiments, culturing of an embryo starting from the late gastrulation stage is continued also following reaching the hind limb formation stage.

According to specific embodiments, culturing of an embryo starting from the late gastrulation stage is effected for at least 1 day, at least 2 days or at least 3 days.

According to specific embodiments, culturing of an embryo starting from the late gastrulation stage is effected for 3-5 days or 3-4.5 days.

According to specific embodiments, culturing of an embryo starting from the late gastrulation stage is effected for about 4 days.

According to specific embodiments, culturing is from E7.5 to E11-11.5.

The late gastrulation stage embryo of some embodiments of the invention may be obtained by dissecting the embryo out from a uterus of a pregnant female mouse. Methods of obtaining live undamaged embryos (e.g. late gastrulation stage embryos) are well known in the art and are further described in details in the Examples section which follows.

According to specific embodiments, the embryo is dissected from the decidua and parietal yolk sac, leaving the intact ectoplacental cone attached to the egg cylinder. For example, the decidua is isolated from the uterine tissue and the tip of the pear-shaped decidua is cut. The decidua is then opened into halves and the embryo is grasped from the decidua and the parietal yolk sac is peeled off the embryo. According to specific embodiments, embryo dissection is performed at 37° C., within a maximum of 30 minutes.

According to specific embodiments, the embryo is dissected into a dissection medium prior to the culturing. Such a dissection medium may comprise a base medium such as a synthetic tissue culture medium, e.g. DMEM supplemented with salts, nutrients, minerals, vitamins, amino acids, nucleic acids, and/or proteins such as cytokines, growth factors and/or hormones. According to specific embodiments, the dissection medium comprises glucose (e.g. 1 mg/ml). According to specific embodiments, the dissection medium comprises serum (e.g. 10% fetal bovine serum). According to specific embodiments, the dissection medium is equilibrated at 37° C. for at least half an hour prior to use.

According to other specific embodiment, the late gastrulation stage embryo is obtained from a previously cultured embryo. The present inventors have developed novel methods of culturing an embryo from the blastocyst stage until at least the late gastrulation stage (see e.g. Examples 2-3 and 5 of the Examples section which follows).

Thus, according to an additional or an alternative aspect of the present invention, there is provided a method of ex utero culturing a mouse embryo, the method comprising culturing a mouse embryo at a post implantation pre gastrulation to early gastrulation stage in a static culture under conditions that allow development of said embryo to an early somite stage, wherein said conditions comprise an atmosphere comprising −15-40% oxygen; and a medium comprising at least 30% serum, wherein said serum comprises rat serum and human serum, and a base medium comprising at least 1 mg/ml glucose.

In some embodiments, optionally, the medium further comprises knockout serum replacement (KSR) in addition to the rat serum and the human serum.

In some embodiments, optionally, the KSR partially replaces one of either the human serum, the rat serum or partially replaces a quantity of both.

As used herein, the term “post implantation pre gastrulation” in the context of a mouse embryo refers to an embryo following the implanting blastocyst stage and prior to the early gastrulation stage and is characterized by an egg cylinder-shape prior to symmetry breaking. Typically, an embryo of a post implantation pre gastrulation stage is defined as Theiler stages TS7-TS8 (see Theiler stage definition in the emap database).

According to specific embodiments, the post implantation pre gastrulation stage refers to E5-6.

According to specific embodiments, the post implantation pre gastrulation stage refers to E5.5.

As used herein, the term “early gastrulation” in the context of a mouse embryo refers to an embryo following the post implantation pre gastrulation stage and prior to the late gastrulation stage and is characterized by egg cylinder shape with the primitive streak at the posterior side. Typically, an embryo of a early gastrulation stage is defined as Theiler stages TS8-TS10 (see Theiler stage definition in the emap database).

According to specific embodiments, the post implantation pre gastrulation stage refers to E 6-7.

According to specific embodiments, the post implantation pre gastrulation stage refers to E 6.5.

As used herein, the term “early somite” in the context of a mouse embryo refers to an embryo following the late gastrulation stage and prior to the neural tube closure stage and is characterized by the appearance of the somites and formation of the first organs. Typically, an embryo of an early somite stage is defined as Theiler stages TS12-TS13 (see Theiler stage definition in the emap database).

According to specific embodiments, early somite stage refers to E8-9.

According to specific embodiments, early somite stage refers to E8.5.

Embryonic stage and development may be assessed compared to an in-vivo embryo counterpart at the same developmental stage by multiple ways well known in the art, as further described in details hereinabove and below.

For example, E5.5 may be characterized by the following morphology: formation of the egg cylinder-shape, appearance of the ectoplacental cone, Reichert's membrane and pro-amniotic cavity starts to form.

E6.5 may be characterized by the following morphology: embryos are constituted by three cell lineages: the cup-shaped pluripotent epiblast (Epi) and two extra-embryonic lineages, the extraembryonic ectoderm (ExE) and the visceral endoderm (VE). The cavities in the embryonic and extraembryonic compartments are unified to form the pro-amniotic cavity, radial symmetry is broken in the epiblast to initiate specification of the primitive streak.

E8.5 may be characterized by the following morphology: >4 somites, embryo curved dorsally, amnion and yolk sac are enclosing the embryo, the allantois extended into the exocoelom and started to fuse with the chorion, the circulatory system differentiated and blood circulated through the vessels encircling the yolk sac and in the embryo, beating horseshoe-like heart rudiment and foregut pocket visible in the frontal part of the embryo, closing but unfused neural folds.

Culturing of an embryo starting from the post implantation pre gastrulation to early gastrulation stage of some embodiments of the invention may be effected until reaching the early somite stage or any developmental stage therein-between.

According to specific embodiments, culturing of an embryo starting from the post implantation pre gastrulation to early gastrulation stage is effected until reaching the early somite stage.

According to specific embodiments, culturing of an embryo starting from the post implantation pre gastrulation to early gastrulation stage is effected for at least 1 day, at least 2 days, at least 2.5 days or at least 3 days.

According to specific embodiments, culturing of an embryo starting from the post implantation pre gastrulation to early gastrulation stage is effected for 2-3 days.

According to specific embodiments, culturing is from E5.5-6.5 to E8.5.

According to specific embodiments, culturing of the post implantation pre gastrulation to early gastrulation stage is continued also following reaching the early somite stage.

Thus, according to an additional or an alternative aspect of the present invention, there is provided a method of ex-utero culturing a mouse embryo, the method comprising:
(a) culturing a mouse embryo at a post implantation pre gastrulation to early gastrulation stage in a static culture under a first set of conditions that allow development of said embryo to an early somite stage, wherein said first set of conditions comprise an atmosphere comprising −15-40% oxygen; and a first medium comprising at least 30% serum, wherein said serum comprises rat serum and human serum, and a base medium comprising at least 1 mg/ml glucose, so as to obtain an embryo of an early somite stage; and
(b) culturing said embryo of said early somite stage in a dynamic culture under a second set of conditions that allow development of said embryo to a hind limb formation stage, wherein said second set of conditions comprise a hyperbaric pressure of more than 5 and less than 10.2 pounds per square inch (psi); an atmosphere comprising 15-40% oxygen; and a second medium comprising at least 30% serum, wherein said serum comprises rat serum and human serum, and a base medium comprising at least 3 mg/ml glucose.

In some embodiments, optionally, the medium further comprises knockout serum replacement (KSR) in addition to the rat serum and the human serum.

In some embodiments, optionally, the KSR partially replaces one of either the human serum, the rat serum or partially replaces a quantity of both.

According to an additional or an alternative aspect of the present invention, there is provided a method of ex-utero culturing a mouse embryo, the method comprising:
(a) culturing a mouse embryo at a post implantation pre gastrulation to early gastrulation stage in a static culture under a first set of conditions that allow development of said embryo to a late gastrulation to early somite stage, wherein said first set of conditions comprise an atmosphere comprising 15-40% oxygen; and a first medium comprising at least 30% serum, wherein said serum comprises rat serum and human serum, and a base medium comprising at least 1 mg/ml glucose, so as to obtain an embryo of a late gastrulation to early somite stage; and
(b) culturing said embryo of said late gastrulation to early somite stage in a dynamic culture under a second set of conditions that allow development of said embryo to a posterior neuropore closure to hind limb formation stage, wherein said second set of conditions comprise an atmosphere comprising 15-40% oxygen; and a second medium comprising at least 30% serum, wherein said serum comprises rat serum and human serum, and a base medium comprising at least 3 mg/ml glucose; and wherein said second set of conditions comprise a hyperbaric pressure of more than 5 and less than 10.2 pounds per square inch (psi) starting the latest when said embryo reaches said early somite stage.

According to some embodiments of the invention, optionally, the medium further comprises knockout serum replacement (KSR) in addition to the rat serum and the human serum.

According to some embodiments of the invention, optionally, the KSR partially replaces one of either the human serum, the rat serum or partially replaces a quantity of both.

Culturing an embryo starting from the post implantation pre gastrulation to early gastrulation stage of some embodiments of the invention may be effected until reaching the hind limb formation stage or any developmental stage therein-between.

According to specific embodiments, culturing of an embryo starting from the post implantation pre gastrulation to early gastrulation stage is effected until reaching the hind limb formation stage.

According to specific embodiments, culturing of an embryo starting from the post implantation pre gastrulation to early gastrulation stage is effected for at least 1 day, at least 2 days, at least 3 days, at least 4 days, at least 5 days or at least 6 days.

According to specific embodiments, culturing of an embryo starting from the post implantation pre gastrulation to early gastrulation stage is effected for 4-6 or 5-6 days.

According to specific embodiments, culturing of an embryo starting from the post implantation pre gastrulation to early gastrulation stage is effected for 2-3 days so as to obtain an embryo of an early somite stage followed by culturing of the early somite stage embryo for about 3 days.

According to specific embodiments, culturing is from E5.5-6.5 to E11-11.5.

According to specific embodiments, culturing is from E5.5-6.5 to E10.5. According to specific embodiments, culturing of the post implantation pre gastrulation to early gastrulation stage is continued also following reaching the posterior neuropore closure or hind limb formation stage.

Thus, according to an additional or an alternative aspect of the present invention, there is provided a method of ex-utero culturing a mouse embryo, the method comprising culturing a mouse embryo at a post implantation pre gastrulation to early gastrulation stage according to the method disclosed herein so as to obtain said embryo of said posterior neuropore closure to hind limb formation stage; and
culturing said embryo of said posterior neuropore closure to hind limb formation stage in a dynamic culture under a set of conditions (e.g. third set of conditions) that allow development of said embryo to an indented anterior footplate stage, wherein the set of conditions comprise a hyperbaric pressure of more than 5 and less than 10.2 pounds per square inch (psi); an atmosphere comprising 30-95% oxygen; and medium comprising at least 30% serum, wherein said serum comprises rat serum and human serum, and a base medium comprising at least 3 mg/ml glucose.

In some embodiments, optionally, the medium further comprises knockout serum replacement (KSR) in addition to the rat serum and the human serum.

In some embodiments, optionally, the KSR partially replaces one of either the human serum, the rat serum or partially replaces a quantity of both.

Culturing an embryo starting from the post implantation pre gastrulation to early gastrulation stage of some embodiments of the invention may be effected until reaching the indented anterior footplate stage or any developmental stage therein-between.

According to specific embodiments, culturing of an embryo starting from the post implantation pre gastrulation to early gastrulation stage is effected until reaching the indented anterior footplate stage.

According to specific embodiments, culturing of an embryo starting from the post implantation pre gastrulation to early gastrulation stage is effected for at least 6, at least 7 or at least 8 days.

According to specific embodiments, culturing of an embryo starting from the post implantation pre gastrulation to early gastrulation stage is effected for 6-9 or 6-8 days.

According to specific embodiments, culturing of an embryo starting from the post implantation pre gastrulation to early gastrulation stage is effected for 2-3 days so as to obtain an embryo of an early somite stage, followed by culturing of the early somite stage embryo for about 2-3 days so as to obtain an embryo of a posterior neuropore closure to hind limb formation stage, followed by culturing of the posterior neuropore closure to hind limb formation stage embryo for about 2-4 days.

According to specific embodiments, culturing is from E5.5-6.5 to E13.5.

The post implantation pre gastrulation to early gastrulation stage embryo of some embodiments of the invention may be obtained by dissecting the embryo out from a uterus of a pregnant female mouse. Methods of obtaining live undamaged embryos (e.g. embryos at a post implantation pre gastrulation to early gastrulation stage) are well known in the art and are further described in details hereinabove and in the Examples section which follows.

According to other specific embodiment, the post implantation pre gastrulation to early gastrulation stage embryo is obtained from a previously cultured embryo. The present inventors have developed novel methods of culturing an embryo from the implanting blastocyst stage until at least the hind limb formation stage (see Example 5 of the Examples section which follows).

Thus, according to an additional or an alternative aspect of the present invention, there is provided a method of ex-utero culturing a mouse embryo, the method comprising culturing a mouse embryo at an implanting blastocyst stage in a static culture under conditions that allow development of said embryo to a post implantation pre gastrulation stage, wherein said conditions comprise an atmosphere comprising −15-40% oxygen; and a medium comprising 15-75% serum and a base medium comprising Insulin-Transferrin-Selenium-Ethanolamine (ITS-X), progesterone, sodium lactate and 3,3′,5-Triiodo-L-thyronine (T3).

According to an additional or an alternative aspect of the present inventor, there is provided a method of ex-utero culturing a mouse embryo, the method comprising culturing a mouse embryo at an implanting blastocyst stage in a static culture under conditions that allow development of said embryo to a post implantation pre gastrulation stage, wherein said conditions comprise an atmosphere comprising 15-40% oxygen; a medium comprising 15-75% serum; and at least one of the following:
(i) an incision in said implanting blastocyst to release fluid and tension from within said blastocyst cavity is made prior to said culturing;
(ii) said serum is provided in said medium in increasing concentrations throughout said culturing; and/or
(iii) said serum comprises a human serum for at least part of said culturing.

As used herein, the term “implanting blastocyst” in the context of a mouse embryo refers to an embryo following a 64 cells blastocyst stage and prior to post implantation pre gastrulation stage and is characterized by segregation of the primitive endoderm and epiblast in the inner cell mass. Typically, an embryo of implanting blastocyst stage is defined as Theiler stages TS5-6 (see Theiler stage definition in the emap database).

According to specific embodiments, implanting blastocyst stage refers to E4-5.

According to specific embodiments, implanting blastocyst stage refers to E4.5.

Embryonic stage and development may be assessed compared to an in-vivo embryo counterpart at the same developmental stage by multiple ways well known in the art, as further described in details hereinabove and below.

For example, E4.5 may be characterized by the following morphology: Cells forming an outer trophectoderm (TE, trophoblast) layer, an inner cell mass (ICM, embryo blast) and a blastocoel (fluid-filled cavity). The primitive endoderm and epiblast are segregated inside the inner cells mass.

According to specific embodiments, the zona pellucida is removed prior to or during the culturing, e.g. at E4.5 e.g. using acidic Tyrode's.

Culturing of an embryo starting from the implanting blastocyst stage of some embodiments of the invention may be effected until reaching the post implantation late gastrulation pre-gastrulation stage or any developmental stage therein-between.

According to specific embodiments, culturing of an embryo starting from the implanting blastocyst stage is effected until reaching the post implantation late gastrulation pre-gastrulation stage.

According to specific embodiments, culturing of an embryo starting from the implanting blastocyst stage is effected for at least 1 day, at least 2 days, at least 3 days or at least 4 days According to specific embodiments, culturing of an embryo starting from the implanting blastocyst stage is effected for 1-5 or 2-4 days.

According to specific embodiments, culturing of an embryo starting from the implanting blastocyst stage is effected for about 3 days.

According to specific embodiments, culturing is from E4.5 to E5.5.

According to specific embodiments, culturing of the implanting blastocyst stage is continued also following reaching the post implantation pre-gastrulation stage.

Thus, according to an additional or an alternative aspect of the present invention, there is provided method of ex-utero culturing a mouse embryo, the method comprising:
(a) culturing a mouse embryo at an implanting blastocyst stage according to the method disclosed herein so as to obtain said embryo of said post implantation pre gastrulation stage; and
(b) culturing said embryo of said post implantation pre gastrulation stage in a static culture under a second set of conditions that allow development of said embryo to a late gastrulation stage, wherein said second set of conditions comprise an atmosphere comprising 15-40% oxygen; and a second medium comprising at least 30% serum, wherein said serum comprises rat serum and human serum, and a base medium comprising at least 1 mg/ml glucose.

In some embodiments, optionally, the medium further comprises knockout serum replacement (KSR) in addition to the rat serum and the human serum.

In some embodiments, optionally, the KSR partially replaces one of either the human serum, the rat serum or partially replaces a quantity of both.

Culturing an embryo starting from the implanting blastocyst stage of some embodiments of the invention may be effected until reaching the late gastrulation stage or any developmental stage therein-between.

According to specific embodiments, culturing of an embryo starting from the implanting blastocyst stage is effected until reaching the late gastrulation stage.

According to specific embodiments, culturing of an embryo starting from the implanting blastocyst stage is effected for at least 5, at least 4 days, at least 5 days or at least 6 days.

According to specific embodiments, culturing of an embryo starting from the implanting blastocyst stage is effected for 3-7, 4-7, 5-7, 5-6 or 6-7 days.

According to specific embodiments, culturing of an embryo starting from the implanting blastocyst stage is effected for about 3 days so as to obtain an embryo of a post implantation pre gastrulation stage followed by culturing of the post implantation pre gastrulation stage embryo for about 2 days.

According to specific embodiments, culturing is from E4.5 to E7.5.

According to specific embodiments, culturing of the implanting blastocyst stage is continued also following reaching the late gastrulation stage.

Thus, according to an additional or an alternative aspect of the present invention, there is provided a method of ex-utero culturing a mouse embryo, the method comprising culturing mouse embryo at an implanting blastocyst stage according to the method disclosed herein so as to obtain said embryo of said late gastrulation stage; and
culturing said embryo of said late gastrulation stage in a dynamic culture under a set of conditions (e.g. third set of conditions) that allow development of said embryo to an early somite stage, wherein the set of conditions comprise an atmosphere comprising 15-40% oxygen; and a medium comprising at least 30% serum, wherein said serum comprises rat serum and human serum, and a base medium comprising at least 1 mg/ml glucose.

According to some embodiments of the invention, optionally, the medium further comprises knockout serum replacement (KSR) in addition to the rat serum and the human serum.

According to some embodiments of the invention, optionally, the KSR partially replaces one of either the human serum, the rat serum or partially replaces a quantity of both.

According to an additional or an alternative aspect of the present invention, there is provided a method of ex-utero culturing a mouse embryo, the method comprising culturing a mouse embryo at an implanting blastocyst stage according to the method disclosed herein so as to obtain said embryo of said post implantation pre gastrulation stage; and
culturing said embryo of said post implantation pre gastrulation stage under a set of conditions (e.g. second set of conditions) that allow development of said embryo to a late gastrulation to early somite stage, wherein the set of conditions comprise an atmosphere comprising 15-40% oxygen; and a medium comprising at least 30% serum, wherein said serum comprises rat serum and human serum, and a base medium comprising at least 1 mg/ml glucose.

In some embodiments, optionally, the medium further comprises knockout serum replacement (KSR) in addition to the rat serum and the human serum.

In some embodiments, optionally, the KSR partially replaces one of either the human serum, the rat serum or partially replaces a quantity of both.

According to specific embodiments, (b) is effected in a static culture, as further described hereinbelow.

According to specific embodiments, (b) is effected in a static culture followed by a dynamic culture, as further described hereinbelow.

According to specific embodiments, (b) is effected in a dynamic culture, as further described hereinbelow.

Culturing an embryo starting from the implanting blastocyst stage of some embodiments of the invention may be effected until reaching the early somite stage or any developmental stage therein-between.

According to specific embodiments, culturing of an embryo starting from the implanting blastocyst stage is effected until reaching the early somite stage.

According to specific embodiments, culturing of an embryo starting from the implanting blastocyst stage is effected for at least 4 days, at least 5 days, at least 6 days or at least 7 days.

According to specific embodiments, culturing of an embryo starting from the implanting blastocyst stage is effected for 4-8, 5-8, 6-8, 6-7 or 7-8 days.

According to specific embodiments, culturing of an embryo starting from the implanting blastocyst stage is effected for about 3 days so as to obtain an embryo of a post implantation pre gastrulation stage followed by culturing of the post implantation pre gastrulation stage embryo for about 2 days so as to obtain an embryo of a late gastrulation stage followed by culturing of the late gastrulation stage embryo for about 1 day.

According to specific embodiments, culturing is from E4.5 to E8.5.

According to specific embodiments, culturing of the implanting blastocyst stage is continued also following reaching the early somite stage.

Thus, according to an additional or an alternative aspect of the present invention, there is provided a method of ex-utero culturing a mouse embryo, the method comprising culturing mouse embryo at an implanting blastocyst stage according to the method disclosed herein so as to obtain said embryo of said early somite stage; and
culturing said embryo of said early somite stage in a dynamic culture under a set of conditions (e.g. fourth set of conditions) that allow development of said embryo to a hind limb formation stage, wherein the set of conditions comprise a hyperbaric pressure of more than 5 and less than 10.2 pounds per square inch (psi); an atmosphere comprising 15-40% oxygen; and a medium comprising at least 30% serum, wherein said serum comprises rat serum and human serum, and a base medium comprising at least 3 mg/ml glucose.

In some embodiments, optionally, the medium further comprises knockout serum replacement (KSR) in addition to the rat serum and the human serum.

In some embodiments, optionally, the KSR partially replaces one of either the human serum, the rat serum or partially replaces a quantity of both.

According to an additional or an alternative aspect of the present invention, there is provided a method of ex-utero culturing a mouse embryo, the method comprising culturing a mouse embryo at an implanting blastocyst stage according to the method disclosed herein so as to obtain said embryo of said late gastrulation to early somite stage; and
culturing said embryo of said late gastrulation to early somite stage in a dynamic culture under a set of conditions (e.g. third set of conditions) that allow development of said embryo to a posterior neuropore closure to hind limb formation stage, wherein the set of conditions comprise a hyperbaric pressure of more than 5 and less than 10.2 pounds per square inch (psi); an atmosphere comprising 15-40% oxygen; and a medium comprising at least 30% serum, wherein said serum comprises rat serum and human serum, and a base medium comprising at least 3 mg/ml glucose.

According to some embodiments of the invention, optionally, the medium further comprises knockout serum replacement (KSR) in addition to the rat serum and the human serum.

According to some embodiments of the invention, optionally, the KSR partially replaces one of either the human serum, the rat serum or partially replaces a quantity of both.

Culturing an embryo starting from the implanting blastocyst stage of some embodiments of the invention may be effected until reaching the hind limb formation stage or any developmental stage therein-between.

According to specific embodiments, culturing of an embryo starting from the implanting blastocyst stage is effected until reaching the hind limb formation stage.

According to specific embodiments, culturing of an embryo starting from the implanting blastocyst stage is effected for at least 6, at least 7 days, at least 8 days or at least 10 days.

According to specific embodiments, culturing of an embryo starting from the implanting blastocyst stage is effected for 6-11, 7-11, 8-11 or 9-11 days.

According to specific embodiments, culturing of an embryo starting from the implanting blastocyst stage is effected for 8-10 days.

According to specific embodiments, culturing of an embryo starting from the implanting blastocyst stage is effected for about 9 days.

According to specific embodiments, culturing of an embryo starting from the implanting blastocyst stage is effected for about 3 days so as to obtain an embryo of a post implantation pre gastrulation stage, followed by culturing of the post implantation pre gastrulation stage embryo for about 2 days so as to obtain an embryo of a late gastrulation stage, followed by culturing of the late gastrulation stage embryo for about 1 day so as to obtain an embryo of an early somite stage, and further followed by culturing of the early smite stage embryo for about 3 days.

According to specific embodiments, culturing is from E4.5 to E11-11.5.

According to specific embodiments, culturing is from E4.5 to E10.5.

According to specific embodiments, culturing of the implanting blastocyst stage is continued also following reaching the posterior neuropore closure to hind limb formation stage.

Thus, according to an additional or an alternative aspect of the present invention, there is provided a method of ex-utero culturing a mouse embryo, the method comprising culturing a mouse embryo at an implanting blastocyst stage according to the method described herein so as to obtain said embryo of said posterior neuropore closure to hind limb formation stage; and
culturing said embryo of said posterior neuropore closure to hind limb formation stage in a dynamic culture under a set of conditions (e.g. fourth set of conditions) that allow development of said embryo to a indented anterior footplate stage, wherein the conditions comprise a hyperbaric pressure of more than 5 and less than 10.2 pounds per square inch (psi); an atmosphere comprising 30-95% oxygen; and a medium comprising at least 30% serum, wherein said serum comprises rat serum and human serum, and a base medium comprising at least 3 mg/ml glucose.

In some embodiments, optionally, the medium further comprises knockout serum replacement (KSR) in addition to the rat serum and the human serum.

In some embodiments, optionally, the KSR partially replaces one of either the human serum, the rat serum or partially replaces a quantity of both.

According to specific embodiments, culturing of an embryo starting from the implanting blastocyst stage is effected for about 1-3 days so as to obtain an embryo of a post implantation pre gastrulation stage, followed by culturing of the post implantation pre gastrulation stage embryo for about 2 days so as to obtain an embryo of a late gastrulation stage, followed by culturing of the late gastrulation stage embryo for about 1 day so as to obtain an embryo of an early somite stage, further followed by culturing of the early somite stage embryo for about 2-3 days so as to obtain an embryo of a posterior neuropore closure to hind limb formation stage, followed by culturing of the posterior neuropore closure to hind limb formation stage embryo for about 2-4 days.

According to specific embodiments, culturing is from E4.5 to E13.5. The implanting blastocyst embryo of some embodiments of the invention may be obtained by isolating the blastocyst from a female mouse. Methods of obtaining live undamaged blastocysts are known in the art and are disclosed for example in e.g. Bedzhov, I. & Zernicka-Goetz, M. Cell (2014). doi:10.1016/j.cell.2014.01.023; Bedzhov I, Leung C Y, Bialecka M, Zernicka-Goetz M. Nat Protoc. 2014 December; 9(12):2732-9, the contents of which are fully incorporated herein by reference.

According to other specific embodiment, the implanting blastocyst stage embryo is obtained from a previously cultured embryo. Several such methods are known in the art and are disclosed in e.g. White, M. D. et al. Cell 165, 75-87 (2016), the contents of which are fully incorporated herein by reference, and in the Examples section which follows, and include culturing of cells following in-vitro fertilization or following zygote isolation until the implanting blastocyst stage [e.g. in a static culture in a Continuous Single Culture Complete (CSCM) medium or a KSOM medium] and optionally removal of the zona pellucida.

According to specific embodiments, an incision is made in the implanting blastocyst to release fluid and tension from within said blastocyst cavity is made prior to or during the culturing. At E4.5 the mural trophectoderm is separated from the epiblast by laser-assisted microdissection, performed at room temperature in M2 medium pre-heated at 37° C. The procedure is done using an inverted microscope with attached micromanipulators and the LYCOS RED-I laser objective, set on “Multipulse” mode. The embryos are held from both the polar and the mural trophectoderm using two micropipettes, and subsequently moved over the cutting laser beam, at the same time that the micropipettes are pull apart to separate the tissues. Collect the epiblast part of the embryo with the mouth pipette for further cultivation. Hence, the method of some embodiments of the invention comprises in-vitro or ex-utero culturing of a mouse embryo from E0 to the hind limb formation stage, or any developmental stage therein-between.

The method of some embodiments of the invention comprises in-vitro or ex-utero culturing of a mouse embryo from E0 to the indented anterior footplate stage, or any developmental stage therein-between.

According to specific embodiments, the method further comprises determining development of the embryo prior to, during and/or following the culturing. Methods of assessing development are well known in the art and are further described in details hereinabove and below.

As used herein, the term “culturing” refers to at least an embryo at the indicated developmental stage and culture medium in an in-vitro or ex-vivo (ex-utero) environment. The culture is maintained under conditions (or set of conditions) capable of inducing development into the embryonic developmental stage(s) disclosed herein. Such conditions include for example an appropriate temperature (e.g., 37° C.), atmosphere (e.g., % O₂, % CO₂), pressure, pH, light, medium, supplements and the like.

The culture may be in a glass, plastic or metal vessel that can provide an aseptic environment for culturing. According to specific embodiments, the culture vessel includes dishes, plates, flasks, bottles, vials, bags, bioreactors or any device that can be used to grow cells.

According to specific embodiments, the culture is maintained under sterile conditions.

According to specific embodiments, the culture is maintained at 37-38° C.

According to specific embodiments, the culture is maintained at 38° C.

According to specific embodiments, the culture is maintained at 37° C.

As changes in temperature may affect embryo developments, according to specific embodiments, care should be taken not to keep the embryo in a temperature higher than 38° C. and lower than 35° C. for a long periods of time. Thus, for example, opening the culture incubator or keeping the embryo at room temperature for a long periods of time should be avoided.

According to specific embodiments, the culture is a static culture.

According to other specific embodiments, the culture is a dynamic culture.

According to specific embodiments, the culture is a static culture followed by a dynamic culture.

As used herein, the term “static culture” refers to a cell culture that is carried out without agitation of the culture.

According to specific embodiments, the static culture is effected at least until the embryo reaches a post implantation pre gastrulation stage.

According to specific embodiments, the static culture is effected at least until the embryo reaches a early gastrulation stage.

According to specific embodiments, the static culture is effected at least until the embryo reaches a late gastrulation stage.

According to specific embodiments, the static culture ends the latest when the embryo reaches an early somite stage.

According to specific embodiments, to prevent sticking of the embryonic epiblast and yolk sac to the culture vessel during the static culture, the culture is examined to ensure that only the ectoplacental cone remains attached to the surface of the plate. According to specific embodiments, the embryos are carefully pushed away from the plate surface by using e.g. forceps, when needed.

As used herein, the term “dynamic culture” refers to a cell culture that is carried out with agitation (e.g. rolling, shaking, inverting) of the culture. To reiterate in a dynamic culture the whole culture, including the embryo, is agitated. Non-limiting examples of dynamic cultures include a roller culture (a culture on a rolling device), a shaker culture (a culture on a shaker, e.g. orbital shaker).

According to specific embodiments, the dynamic culture is a roller culture.

According to specific embodiments, the rolling culture is rolled in 30 rpm.

Rotator culture units may be obtained commercially from e.g. B.T.C. Engineering,—Cullum Starr Precision Engineering Ltd—UK.

According to other specific embodiments, the dynamic culture is a shaker culture.

According to specific embodiments, the shaker rotates at 30-80 rpm, 40-70 rpm, 50-70 rpm or 55-65 rpm.

According to a specific embodiment, the shaker rotates at about 60 rpm.

According to specific embodiments, the dynamic culture starts the latest when the embryo reaches an early somite stage.

As used herein the phrase “culture medium” refers to a liquid substance used to support the growth of an embryo and optionally induce their development. The culture medium used according to some embodiments of the invention can be a water-based medium which includes a combination of substances such as salts, nutrients, minerals, vitamins, amino acids, nucleic acids, and/or proteins such as cytokines, growth factors and hormones, all of which are needed for cell growth an d embryo development.

Preferably, all ingredients included in the culture medium of the present invention are substantially pure, i.e., a tissue culture grade.

For example, the culture medium may comprise a base medium such as a synthetic tissue culture medium, e.g. DMEM, DMEM/F12 or advanced DMEM/F12 (can be commercially obtained from e.g. GIBCO® or Biological Industries), KO-DMEM (can be commercially obtained from e.g. GIBCO®), CMRL (can be commercially obtained from e.g. GIBCO®), TCM199 (can be commercially obtained from e.g. Sigma), StemPro® (can be commercially obtained from e.g. Thermo Fisher Scientific), RPMI (can be commercially obtained from e.g. Biological Industries) or a combination thereof supplemented with the necessary additives as is further described herein.

According to specific embodiments, the base medium comprises DMEM or DMEM/F12.

According to specific embodiments, the base medium comprises CMRL.

According to specific embodiments, the base medium comprises TCM199. According to specific embodiments, the base medium is devoid of phenol red.

According to a specific embodiment, the base medium comprises DMEM having the same components as the DMEM of GIBCO® Cat. No. 11880.

According to a specific embodiment, the base medium comprises DMEM/F12 having the same components as the DMEM/F12 of GIBCO® Cat. No. 12634-010.

According to a specific embodiment, the base medium comprises CMRL having the same components as the CMRL of GIBCO® Cat. No. 11530037.

According to a specific embodiment, the base medium comprises TCM199 having the same components as the TCM199 of Sigma Cat. No. M4530.

According to specific embodiments, the culture medium comprises serum.

According to specific embodiments, the culture medium comprises 10-80% 15-80%, 20-80%, 15-75% or 20-75% [volume/volume (v/v)] serum.

According to specific embodiments, the culture medium comprises 15-75% (v/v) serum.

According to specific embodiments, the culture medium comprises 15-60%, 15-40% or 15-30% (v/v) serum.

According to specific embodiments, the culture medium comprises 15-60%, 15-40% (v/v) serum.

According to a specific embodiment, the culture medium comprises 20-40% (v/v) serum.

According to a specific embodiment, the culture medium comprises 20-30% (v/v) serum.

According to other specific embodiments, the culture medium comprises at least 20% (v/v) serum.

According to a specific embodiment, the culture medium comprises about 20% (v/v) serum.

According to a specific embodiment, the culture medium comprises about 30% (v/v) serum.

According to other specific embodiments, the culture medium comprises at least 30% (v/v) serum.

According to other specific embodiments, the culture medium comprises at least 35% (v/v), at least 40% (v/v), at least 45% (v/v), at least 50% (v/v), at least 55% (v/v), at least 60% (v/v), at least 65% (v/v), at least 70% (v/v) serum.

According to other specific embodiments, the culture medium comprises at least 50% (v/v) serum.

According to other specific embodiments, the culture medium comprises 40-80%, 50-80%, 60-80%, 70-80% (v/v) serum.

According to other specific embodiments, the culture medium comprises 70-80% (v/v) serum.

According to a specific embodiment, the culture medium comprises about 75% (v/v) serum.

According to a specific embodiment, the culture medium comprises increasing serum concentrations throughout the culturing.

According to a specific embodiment, the culture medium comprises increasing serum concentrations throughout the static culture.

According to specific embodiments, the serum is provided in the medium in increasing concentrations throughout the static culture followed by a constant concentrations throughout the dynamic culture.

Increasing the serum concentrations may be effected for example every 12-72 hours, every 12-60 hours, every, every 16-52 hours or every 24-48 hours.

According to a specific embodiment, the increasing serum concentrations is effected every 16-52 hours of the culturing.

The serum may be obtained from a rodent (e.g. rat, mouse) or a mammal (e.g. bovine, human).

According to specific embodiments, care should be taken that the serum (e.g. human serum) is devoid of any traces of hemolysis.

According to specific embodiments, the serum is obtained from an adult animal.

According to other specific embodiments, the serum is obtained from a fetal animal.

According to specific embodiments, the serum comprises a cord blood serum. Methods of obtaining cord blood serum (e.g. human cord blood serum) are well known in the art and are further described in the Examples section which follows.

According to specific embodiments, the serum comprises bovine serum (e.g. FCS).

According to specific embodiments, the serum comprises rat serum.

According to specific embodiments, the serum comprises human serum.

According to specific embodiments, the serum comprises human serum for at least part of the culturing.

According to specific embodiments, the human serum comprises umbilical cord serum (HCS).

According to other specific embodiments, the human serum comprises adult blood serum (HBS).

According to specific embodiments, the serum comprises rat serum and human serum.

According to specific embodiments, the ratio between the rat serum and the human serum in the medium is between 1:1-5:1 (v/v).

According to specific embodiments, the ratio between the rat serum and the human serum in the medium is between 1:1-3:1 (v/v).

According to specific embodiments, the ratio between the rat serum and the human serum in the medium is about 2:1 (v/v).

According to specific embodiments, the ratio between the rat serum and the human serum in the medium is 2:1 (v/v).

In some embodiments, optionally, the medium further comprises knockout serum replacement (KSR) in addition to the rat serum and the human serum.

In some embodiments, optionally, the KSR partially replaces one of either the human serum, the rat serum or partially replaces a quantity of both.

According to specific embodiments, the ratio between the serum and the base medium in the culture medium is between 1:0.5-10:1, 1:1-10:1 or 1:1-8:1 (v/v).

According to specific embodiments, the ratio between the serum and the base medium in the culture medium is between 1:1-5:1 (v/v).

According to specific embodiments, the ratio between the serum and the base medium in the culture medium is about 3:1 (v/v).

According to specific embodiments, the ratio between the serum and the base medium in the culture medium is 3:1 (v/v).

According to specific embodiments, the culture medium comprises 20-30% base medium, 40-60% rat serum and 20-30% human serum (v/v).

According to a specific embodiment, the culture medium comprises 25% base medium, 50% rat serum and 25% human serum (v/v).

According to specific embodiments, the serum is heat inactivated (e.g. in 55° C. 30-45 minutes).

According to specific embodiments, the serum is added to the culture medium just prior to use.

According to some embodiments of the invention, the culture medium can further include antibiotics (e.g., PEN-STREP), L-glutamine (e.g., GlutaMAX™), sodium pyruvate, HEPES.

According to some embodiments of the invention, the culture medium can further include NEAA (non-essential amino acids).

According to specific embodiments, the medium comprises glucose.

According to specific embodiments, the medium of the base medium comprises at least 1 mg/ml, at least 2 mg/ml, at least 3 mg/ml or at least 4 mg/ml glucose.

According to specific embodiments, the medium or the base medium comprises at least 1 mg/ml glucose.

According to specific embodiments, the medium or the base medium comprises at least 3 mg/ml glucose.

According to specific embodiments, the medium or the base medium comprises at least 4 mg/ml, at least 5 mg/ml, at least 6 mg/ml, at least 7 mg/ml or at least 8 mg/ml glucose.

According to specific embodiments, the medium or the base medium comprises at least 4 mg/ml glucose.

According to specific embodiments, the medium or the base medium comprises 2-12 mg/ml glucose, 3-12 mg/ml glucose, 4-12 mg/ml glucose or 4-8 mg/ml glucose.

According to specific embodiments, the glucose is provided in the medium in a constant or increasing concentrations throughout the culturing.

Thus, according to specific embodiments, throughout the culturing there is no decrease in the glucose concentration provided in the medium throughout the culturing (e.g. while passing from one set of conditions to a following set of conditions).

According to specific embodiments, the glucose is provided in the medium in a constant concentration throughout the culturing.

According to specific embodiments, the glucose is provided in the medium in a constant concentration throughout the static culture.

According to specific embodiments, the glucose is provided in the medium in increasing concentrations throughout the culturing.

According to specific embodiments, the glucose is provided in the medium in increasing concentrations throughout the dynamic culture.

According to specific embodiments, the glucose is provided in the medium in a constant concentration throughout the static culture followed by increasing concentrations throughout the dynamic culture.

According to specific embodiments, the glucose is provided in the medium or the base medium in increasing concentrations throughout the culturing starting from at least 1 mg/ml up to 4-5 mg/ml.

According to specific embodiments, the glucose is provided in the medium or the base medium in increasing concentrations throughout the culturing starting from at least 3 mg/ml up to 4-5 mg/ml.

According to specific embodiments, the glucose is provided in the medium or the base medium in increasing concentrations throughout the culturing starting from at least 1 mg/ml up to 12 mg/ml.

According to specific embodiments, the medium or the base medium comprises at least 1 mg/ml glucose up to an early somite stage and at least 3 mg/ml glucose when the embryo reaches the somite stage onwards.

According to specific embodiments, the increasing is effected by 1.1-2.5 fold, 1.1-2 fold or 1.1-1.5 fold in every step of the increasing.

According to specific embodiments, the increasing is effected every 0.5-2 days, every 0.5-1.5, every 1-2, or every 1-1.5 days of the culturing.

According to specific embodiments, the increasing is effected every 20-28 hours of the culturing.

Thus, for example, according to specific embodiments, the medium or the base medium comprises 1 mg/ml glucose up to an early somite stage, followed by 3-4 mg/ml the following day followed by 3.5-4.5 mg/ml the following day, followed by 4-5 mg/ml the following day.

According to specific embodiments, the medium or the base medium comprises at least 5 mg/ml or at least 6 mg/ml glucose when the embryo reaches the posterior neuropore closure stage onwards.

According to specific embodiments, the medium or the base medium comprises at least 6 mg/ml, at least 7 mg/ml or at least 8 mg/ml glucose when the embryo reaches the hind limb formation stage onwards.

As further described hereinbelow, the present inventors have identified novel culture media comprising specific factors which can be used to allow development of an embryo to organogenesis or any developmental stage therein-between (see the Examples section which follows).

Hence, the present invention also envisages aspects related to media as described in the Examples section which follows, wherein components of the media are provided in concentrations of ±20%.

According to specific embodiments, any of the media may further comprise additional supplements including, but not limited to, antibiotics (e.g., PEN-STREP), L-glutamine (e.g., GlutaMAX™), non-essential amino acids (NEAA), Insulin-Transferrin-Selenium-Ethanolamine (ITS-X), β-Estradiol, progesterone, N-acetyl L-Cysteine, 3,3′,5-Triiodo-L-thyronine sodium salt (T3), sodium lactate, sodium pyruvate, glucose (e.g. at least 1 mg/ml, at least 3 mg/ml), serum replacement (e.g. KSR) and/or HEPES, as further described hereinabove and below.

According to specific embodiments, the medium or the base medium comprises a component selected from the group consisting of progesterone, estrogen, N2, N27, Insulin-Transferrin-Selenium-Ethanolamine (ITS-X), and 3,3′,5-Triiodo-L-thyronine sodium salt (T3).

For example, the present inventors have identified a novel culture medium comprising specific factors which can be used to allow development of an implanting blastocyst stage mouse embryo to a post implantation pre gastrulation stage (see e.g. Example 5 of the Examples section which follows).

Hence, according to an aspect of some embodiments of the invention, there is provided a culture medium comprising a medium comprising Insulin-Transferrin-Selenium-Ethanolamine (ITS-X), progesterone, 3,3′,5-Triiodo-L-thyronine (T3) and optionally sodium lactate.

According an aspect of some embodiments of the invention, there is provided a culture medium comprising a medium comprising Insulin-Transferrin-Selenium-Ethanolamine (ITS-X), progesterone, sodium lactate and 3,3′,5-Triiodo-L-thyronine (T3).

According to specific embodiments, the ITS-X is provided in the medium or the base medium in a concentration of 1×.

According to specific embodiments, the progesterone is provided in the medium or the base medium in a concentration of 200 ng/ml.

According to specific embodiments, the progesterone is provided in the medium or the base medium in a concentration of 20 ng/ml.

According to specific embodiments, the T3 is provided in the medium or the base medium in a concentration of 100 nM.

According to specific embodiments, the medium further comprises N2 and B27.

According to specific embodiments, the conditions comprise N2 and/or B27 in the base medium following 1, 2 or 3 days of the culturing.

According to specific embodiments, the conditions comprise N2 and/or B27 in the base medium following 2 days of the culturing.

According to specific embodiments, the N2 is provided in the medium or the base medium in a concentration of 1×.

According to specific embodiments, the B27 is provided in the medium or the base medium in a concentration of 0.5×.

According to other specific embodiments, the culture medium is devoid of N2 and/or B27.

According to specific embodiments, the culture conditions comprise a medium devoid of N2 and B27 for a predetermined period of time followed by a medium comprising N2 and B27 for a predetermined period of time (e.g. following 2 days of the culturing). Thus, for example, according to a specific embodiment, culturing of an implanting blastocyst stage mouse embryo is effected for 1-2 days in the absence of N2 and B27 followed by culturing in the presence of N2 and/or B27.

According to specific embodiments, the medium or the base medium further comprises β-estradiol and/or N-acetyl-L-cysteine.

According to specific embodiments, the estradiol is provided in the medium or the base medium in a concentration of 8 nM.

According to specific embodiments, the N-acetyl-L-cysteine is provided in the medium or the base medium in a concentration of 25 mM.

According to specific embodiments, the N-acetyl-L-cysteine is provided in the medium or the base medium in a concentration of 25 μM.

According to specific embodiments, the culture medium is devoid of MATRIGEL®.

According to specific embodiments, the culture medium or the base medium further comprises sodium pyruvate.

According to specific embodiments, the sodium pyruvate is provided in the medium or the base medium in a concentration of at least 0.1 mg/ml, at least 0.12 mg/ml, at least 0.13 mg/ml, at least 0.14 mg/ml, at least 0.15 mg/ml, 0.16 mg/ml, 0.17 mg/ml, 0.18 mg/ml, 0.19 mg/ml, 0.2 mg/ml, 0.21 mg/ml, 0.22 mg/ml.

According to specific embodiments, the sodium pyruvate is provided in the medium or the base medium in a concentration of at least 1 mM, at least 1.5 mM or at least 2 mM.

According to a specific embodiment, the sodium pyruvate is provided in the medium or the base medium in a concentration of about 2 mM.

Non-limiting examples of specific media that can be used are provided in the Examples section which follows, the contents of which represent an integral part of the specification.

According to specific embodiments, the conditions comprise replacement of at least half of the medium every 1-2 days of the culturing.

According to specific embodiments, the conditions comprise replacement of at least half of the medium every 20-28 hours of the culturing.

According to specific embodiments, wherein the culture is a static culture the conditions comprise replacement of at least half of the medium every 1-2 days of the culturing.

According to specific embodiments, wherein the culture is a static culture the conditions comprise replacement of at least half of the medium every 20-28 hours of the culturing.

According to specific embodiments, the conditions comprise replacement of all the medium every 1-2 days of the culturing.

According to specific embodiments, the conditions comprise replacement of all the medium every 20-28 hours of the culturing.

According to specific embodiments, wherein the culture is a dynamic culture the conditions comprise replacement of all the medium every 1-2 days of the culturing.

According to specific embodiments, wherein the culture is a dynamic culture the conditions comprise replacement of all the medium every 20-28 hours of the culturing.

According to specific embodiments, the culture is maintained under a hyperbaric pressure.

According to specific embodiments, the dynamic culture is maintained under a hyperbaric pressure.

According to specific embodiments, the roller culture is maintained under a hyperbaric pressure.

According to specific embodiments, the culture is maintained under a hyperbaric pressure starting the latest when the embryo reaches an early somite stage.

As used herein, the term “hyperbaric pressure” refers to a pressure greater than atmospheric pressure, wherein atmospheric pressure is generally regarded as 14.7 pounds per square inch (psi). Hence, wherein a specific hyperbaric pressure is indicated herein, it refers to the indicated pressure above the atmospheric pressure and not the value indicated per-se. For example, a hyperbaric pressure of 5 psi refers to a pressure of 19.7 psi, a hyperbaric pressure of 6.5 psi refers to a pressure of 21.2 psi and a hyperbaric pressure of 10.2 refers to a pressure of 24.7 psi.

According to specific embodiments, the hyperbaric pressure is more than 2.5 psi, more than 4 psi, more than 5 psi, more than 6 psi.

According to specific embodiments, the hyperbaric pressure is more than 5 psi.

According to specific embodiments, the hyperbaric pressure is less than 10.2 psi, less than 9 psi, less than 8 psi, less than 7 psi.

According to specific embodiments, the hyperbaric pressure is less than 10.2 psi.

According to specific embodiments, the hyperbaric pressure is more than 5 psi and less than 10.2 psi.

According to specific embodiments, the hyperbaric pressure is 6-7 psi.

According to specific embodiments, the hyperbaric pressure is 6.5 psi.

According to other specific embodiments, the culture is maintained under atmospheric pressure.

According to specific embodiments, the static culture is maintained under atmospheric pressure.

According to specific embodiments, the culture is maintained in an atmosphere comprising a controlled level of O₂, N₂ and/or CO₂.

According to specific embodiments, the culturing is effected in an atmosphere comprising 5% CO₂.

According to specific embodiments, the culturing is effected in an atmosphere comprising 5-40% oxygen.

According to specific embodiments, the culturing is effected in an atmosphere comprising 5-30%, 5-25% or 5-21% oxygen.

According to specific embodiments, the culturing is effected in an atmosphere comprising 10-40%, 10-30% or 15-30% oxygen.

According to specific embodiments, the culturing is effected in an atmosphere comprising 15-30% oxygen.

According to specific embodiments, the culturing is effected in an atmosphere comprising 19-23% oxygen.

According to specific embodiments, the culturing is effected in an atmosphere comprising 21% oxygen.

According to specific embodiments, the culturing is effected in an atmosphere comprising 30-95% oxygen.

According to specific embodiments, the culturing is effected in an atmosphere comprising 40-95%, 50-95%, 60-95%, 70-95%, 80-95%, 85-95% or 90-95% oxygen.

According to a specific embodiment, the culturing is effected in an atmosphere comprising 95% oxygen. According to specific embodiments, the culturing is effected in an atmosphere comprising constant or increasing oxygen concentrations throughout the culturing.

Thus, according to specific embodiments, throughout the culturing there is no decrease in the oxygen concentration throughout the culturing (e.g. while passing from one set of conditions to a following set of conditions).

According to specific embodiments, the culturing is effected in an atmosphere comprising increasing oxygen concentrations throughout the culturing starting from 5% up to 15-40%.

According to specific embodiments, the culturing is effected in an atmosphere comprising increasing oxygen concentrations throughout the culturing starting from 5% up to 20-25%.

According to specific embodiments, the culturing is effected in an atmosphere comprising increasing oxygen concentrations throughout the culturing starting from 5% up to 21% a.

According to specific embodiments, the culturing is effected in an atmosphere comprising increasing oxygen concentrations throughout the culturing starting from 15-40% up to 30-95%.

According to specific embodiments, the culturing is effected in an atmosphere comprising increasing oxygen concentrations throughout the culturing starting from 15-40% (e.g. 21%) up to 95%.

According to specific embodiments, the increasing is effected by 1.5-2.5 fold or 1.5-2 fold in every step of the increasing.

According to specific embodiments, the increasing is effected every 0.5-2 days, every 0.5-1.5, every 1-2, or every 1-1.5 dais of the culturing.

According to specific embodiments, the increasing is effected every 20-28 hours of the culturing.

Thus, for example, according to specific embodiments, culturing is effected in 5-10% oxygen on the first day of the culturing, 10-15% oxygen on the second day of the culturing, 15-20% on the third day of the culturing and 20-25% oxygen on the fourth day of the culturing onwards.

According to specific embodiments, culturing is effected in 5% oxygen on the first day of the culturing, 13% oxygen on the second day of the culturing, 18% on the third day of the culturing and 21% oxygen on the fourth day of the culturing onwards.

According to a specific embodiment, the dynamic culture is effected in 5% oxygen on the first day of the culturing, 13% oxygen on the second day of the culturing, 18% on the third day of the culturing and 21% oxygen on the fourth day of the culturing onwards.

Whilst reducing specific embodiments of the present invention to practice, the present inventors have now also developed a robust embryo culture system that faithfully recapitulates for the first time rabbit in utero development from two cells embryo to advanced organogenesis stages, enabling the application and monitoring of external and internal manipulations in rabbit embryos over up to 12-13 days of post-conception development.

As is illustrated hereinunder and in the examples section, which follows, the present inventors show ex utero rabbit embryo culture platforms, that enable appropriate development of embryos from the two cell embryo stage until the three cerebral vesicles (GD11-12) (Example 8). Specifically, gastrulating embryos (GD9) or somitogenesis embryos are grown in 3D rotating bottles settings, while extended culture from the two cell stage requires a combination of novel static and rotating bottle culture protocols.

Thus, according to an aspect of the present invention, there is provided a method of ex-utero culturing a rabbit embryo, the method comprising culturing a mouse embryo at a somitogenesis to early organogenesis stage in a dynamic culture under conditions that allow development of said embryo to a three cerebral vesicles stage, wherein said conditions comprise hyperbaric pressure of more than 5 and less than 10.2 pounds per square inch (psi); an atmosphere comprising 15-40% oxygen; and a medium comprising at least 30% serum, wherein said serum comprises rabbit serum and human serum.

In some embodiments, optionally, the medium further comprises knockout serum replacement (KSR) in addition to the rabbit serum and the human serum.

In some embodiments, optionally, the KSR partially replaces one of either the human serum, the rabbit serum or partially replaces a quantity of both.

As used herein, the term “somitogenesis” in the context of a rabbit embryo refers to an embryo following late gastrulation stage and prior to the early organogenesis stage and is characterized by the appearance of the first one to five somites distinguishable by bright field microscopy.

According to specific embodiments, the somitogenesis stage refers to gestation day (GD) 8-9.

According to specific embodiments, the somitogenesis stage refers to gestation day (GD) 9.

As used herein, the term “gestation day (GD)” in the context of a rabbit embryo refers to an embryo having developmental characteristic of an in vivo (in-uterine tube or in utero, depending on the day) rabbit embryo counterpart at the specified day following mating, wherein GD0 is considered following successful mating.

As used herein, the term “early organogenesis” in the context of a rabbit embryo refers to an embryo following the somitogenesis stage and prior to the appearance of the heart beat stage and is characterized by formation of the neural tube and mesoderm migration.

According to specific embodiments, early organogenesis refers to GD9-10. As used herein, the term “three cerebral vesicles” in the context of a rabbit embryo refers to an embryo following the Late organogenesis stage and prior to the growth stage and is characterized by and exponential expansion of the primordium of organs and maturation.

According to specific embodiments, three cerebral vesicles refers to GD11-12.

According to specific embodiments, three cerebral vesicles refers to GD12.

According to specific embodiments, indented anterior footplate stage refers to embryonic

Embryonic stage and development may be assessed compared to an in vivo embryo counterpart at the same developmental stage by multiple ways including, but not limited to, morphology, length, weight, weight two times, expression of developmental marker genes (e.g. Oct4, Nanog, Sox2, Klf4, Cdx2, Gata4, Gata6, Brachyury, Otx2, Fgf5) using specific antibodies or primers, transcriptional profiling and the like, as further described hereinbelow and in the Examples section which follows which serve as an integral part of the specification.

Morphology assessment of embryonic development may be performed by previously established morphological features, such as described in e.g. S. Beaudoin et al., (2003) Fetal Diagn Ther 18:422-427. Thus, for example, GD1 may be characterized by two-cell stage; GD2 may be characterized by 4-cell stage; GD3 may be characterized by morlula stage, GD4 may be characterized by early blastocyst stage; GD5 may be characterized by blastocyst expansion; GD6 may be characterized by expanded blastocyst; GD7 may be characterized by early gastrulation; GD8 may be characterized by late gastrulation and somitogenesis; GD9 may be characterized by somitogenesis and early organogenesis; GD10 may be characterized by dorsal curvature rostral limb; GD11 may be characterized by appearance of the caudal limb and four faringeal arches; GD12 may be characterized by three cerebral vesicles and optic plate.

Developmental markers can be detected using immunological techniques well known in the art [described e.g. in Thomson J A et al., (1998). Science 282: 1145-7]. Examples include, but are not limited to, immunostaining, microscopy, flow cytometry, western blot, and enzymatic immunoassays. Other non-limiting methods include PCR analysis, RNA fluorescence in situ hybridization (FISH), northern blot, single cell RNA sequencing.

Culturing of an embryo starting from the somitogenesis to early organogenesis stage embryo of some embodiments of the invention may be effected until reaching the three cerebral vesicles stage or any developmental stage therein-between.

According to specific embodiments, culturing of an embryo starting from the somitogenesis to early organogenesis stage is effected until reaching the three cerebral vesicles stage.

According to specific embodiments, culturing of an embryo starting from the somitogenesis to early organogenesis stage is effected for at least 1 day, at least 2 days or at least 3 days.

According to specific embodiments, culturing is from GD9 to GD12.

The somitogenesis to early organogenesis stage embryo of some embodiments of the invention may be obtained by dissecting the embryo out from a uterus of a pregnant female rabbit. Methods of obtaining rabbit live undamaged embryos are well known in the art and disclosed for example in Ozolins Methods Mol Biol (2019) 1965: 219-233; Vicente at al. Journal of Animal and Veterinary Sciences (2015), 2(5): 47-52; Garcia (2018) New Insights into Theriogenology, IntechOpen, London. 10.5772/intechopen.81089, the contents of which are fully incorporated herein by reference, and are also described in the Examples section which follows.

Form Zygote to Morula stage (GD0.5-3) after humanitarian euthanasia, a midline abdominal incision is performed and the distal end of the uterus horns are located and clamped 0.5 cm proximal to the utero-tubal junction the proximal site is separated from the horn the fallopian tube is dissected by blunt dissection until the fimbriae, the tissue is taken to a 10 cm petri dish filled with pre-warmed M2 medium, the clamped zone is removed and using a 10 ml syringe with a 21 g needle filled with M2 medium the fallopian tube is flushed making sure the medium flows though the full tissue. Embryos are collected from the plate using a 10 ul pipette and a stereoscope.

GD6 (protocol not mentioned in literature we found better survival of the embryos with this method compared to flushing the uterus) after humanitarian euthanasia, a midline abdominal incision is performed, and the distal end of the uterus horns are located and clamped 0.5 cm proximal to the utero-tubal junction. The uterine horn is dissected and taken to a 10 cm petri dish and using spring scissors the horn is opened exposing the endometrium, embryos are collected by gently grasping them for the endometrium with forceps.

GD7 (method also not motioned in literature for keeping extraembryonic membranes intact) after humanitarian euthanasia, a midline abdominal incision is performed, and the distal end of the uterus horns are located and clamped 0.5 cm proximal to the utero-tubal junction, the horn is separated from the fallopian tube and each implantation site is dissected separately leaving 3 mm of uterus at each side to avoid damaging the embryo. All the implantation sites are collected in prewarmed dissection medium. To each implantation site an incision is performed in the mesometerial side following the uterus lumen close to the endometrium to avoid damage to the embryo, once open, carefully the embryo is detached form the endometrium.

GD9 (Method form Valerie A marshall Developmental Toxicology 2012 vol 889), after humanitarian euthanasia, a midline abdominal incision is performed, and the distal end of the uterus horns are located and clamped 0.5 cm proximal to the utero-tubal junction, the horn is separated from the fallopian tube and each implantation site is dissected separately leaving 3 mm of uterus at each side to avoid damaging the embryo. All the implantation sites are collected in prewarmed dissection medium on a 10 cm petri dish with sylgard elastomer. The implantation sites are pinned to the plate in both ends and a cut is performed in the antimesometrial side, carefully the embryo is detached from the mesometrial site.

According to specific embodiments, the embryo is dissected into a dissection medium prior to the culturing. Such a dissection medium may comprise a base medium such as a synthetic tissue culture medium, e.g. DMEM supplemented with salts, nutrients, minerals, vitamins, amino acids, nucleic acids, and/or proteins such as cytokines, growth factors and/or hormones. According to specific embodiments, the dissection medium comprises serum (e.g. 10% fetal bovine serum). According to specific embodiments, the dissection medium is equilibrated at 38° C. for at least half an hour prior to use.

According to specific embodiments, the embryo is treated with pronase prior to or during the culturing. Pronase treatment in blastocyst stage is performed to remove the zona pellucida, since protocols of in-vitro culture in mouse and human have proven higher efficiency when this layer is removed. In GD6 pronase treatment has the objective of removing the neozona layer that protects the embryo, in the uterus is removed by enzymes secreted by the uterus and if kept when the embryo reaches GD7 it increases the pressure causing them to break and stop developing, when removed properly embryos can continue their growth. Typically, such a treatment will be effected in GD4 or GD6 according to the respective protocol.

According to other specific embodiment, the somitogenesis to early organogenesis stage embryo is obtained from a previously cultured embryo. The present inventors have developed novel methods of culturing an embryo from the two cells stage until at least the early organogenesis stage (see e.g. Example 8 of the Examples section which follows).

Thus, according to an aspect of the present invention, there is provided a method of ex-utero culturing a rabbit embryo, the method comprising culturing a rabbit embryo at a gastrulation stage in a dynamic culture under conditions that allow development of said embryo to an early organogenesis stage, wherein said conditions comprise an atmosphere comprising 15-40% oxygen; and a medium comprising at least 15% serum, wherein said serum comprises rabbit serum.

As used herein, the term “gastrulation” in the context of a rabbit embryo refers to an embryo following the expanded blastocyst stage and prior to the somitogenesis stage and is characterized by the formation of the primitive streak and epithelial to mesenchymal transition forming three germinal layers.

According to specific embodiments, the gastrulation stage refers to GD6-8.

According to specific embodiments, the gastrulation stage refers to GD6.

Culturing of an embryo starting from the gastrulation stage embryo of some embodiments of the invention may be effected until reaching the early organogenesis stage or any developmental stage therein-between.

According to specific embodiments, culturing of an embryo starting from the gastrulation stage is effected until reaching the early organogenesis stage.

According to specific embodiments, culturing of an embryo starting from the gastrulation stage is effected for at least 1 day, at least 2 days or at least 3 days.

According to specific embodiments, culturing of an embryo starting from the gastrulation stage is effected for about 3 days.

According to specific embodiments, culturing of an embryo starting from the gastrulation stage is effected for about 6 days.

According to specific embodiments, culturing is from GD6 to GD9-10.

According to specific embodiments, culturing of an embryo starting from the gastrulation stage is continued also following reaching the early organogenesis stage.

Thus, according to an additional or an alternative aspect of the present invention, there is provided a method of ex-utero culturing a rabbit embryo, the method comprising culturing a rabbit embryo at a gastrulation stage according to the method disclosed herein so as to obtain said embryo of a somitogenesis to early organogenesis stage; and culturing the embryo at the somitogenesis to early organogenesis stage in a dynamic culture under conditions (e.g. second set of conditions) that allow development of said embryo to a three cerebral vesicles stage, wherein the conditions comprise hyperbaric pressure of more than 5 and less than 10.2 pounds per square inch (psi); an atmosphere comprising 15-40% oxygen; and a medium comprising at least 30% serum, wherein said serum comprises rabbit serum and human serum.

In some embodiments, optionally, the medium further comprises knockout serum replacement (KSR) in addition to the rabbit serum and the human serum.

In some embodiments, optionally, the KSR partially replaces one of either the human serum, the rabbit serum or partially replaces a quantity of both.

Thus, according to specific embodiments, culturing of an embryo starting from the gastrulation stage is effected for 2-3 days so as to obtain and embryo of a somitogenesis to early organogenesis stage, followed by culturing of the somitogenesis to early organogenesis stage embryo for about 3-4 days.

The gastrulation stage embryo of some embodiments of the invention may be obtained by dissecting the embryo out from a uterus of a pregnant female rabbit. Methods of obtaining live undamaged embryos are well known in the art and are further described in details hereinabove and in the Examples section which follows.

According to specific embodiments, culturing is from GD6-7 to GD12.

According to other specific embodiment, the gastrulation stage embryo is obtained from a previously cultured embryo. The present inventors have developed novel methods of culturing an embryo from the two cells stage until at least the gastrulation stage (see e.g. Example 8 of the Examples section which follows).

Thus, according to an additional or an alternative aspect of the present invention, there is provided a method of ex-utero culturing a rabbit embryo, the method comprising culturing a rabbit embryo at a blastocyst stage in a static culture under conditions that allow development of said embryo to a gastrulation stage, wherein said conditions comprise an atmosphere comprising 5-40% oxygen; a medium comprising 15-75% serum, wherein said serum comprises rabbit serum.

As used herein, the term “blastocyst” in the context of a rabbit embryo refers to an embryo following the morula stage and prior to the gastrulation stage and is characterized by the formation of the blastocoel cavity exponential growth and appearance of an inner cell mas and trophectoderm lineages.

According to specific embodiments, the blastocyst stage refers to GD3-5.

According to specific embodiments, the blastocyst stage refers to GD4.

Culturing of an embryo starting from the blastocyst stage of some embodiments of the invention may be effected until reaching the gastrulation stage or any developmental stage therein-between.

According to specific embodiments, culturing of an embryo starting from the blastocyst stage is effected until reaching the gastrulation.

According to specific embodiments, culturing of an embryo starting from the blastocyst stage is effected for at least 1 day, at least 2 days, at least 2.5 days or at least 3 days.

According to specific embodiments, culturing of an embryo starting from the blastocyst stage is effected for 2-3 days.

According to specific embodiments, culturing is from GD4 to GD6-7.

According to specific embodiments, culturing of an embryo starting from the blastocyst stage is continued also following reaching the gastrulation stage.

Thus, according to an additional or an alternative aspect of the present invention, there is provided a method of ex-utero culturing a rabbit embryo, the method comprising culturing a rabbit embryo at a blastocyst stage according to the method disclosed herein so as to obtain said embryo of said gastrulation stage; and
culturing said embryo of said gastrulation stage under a conditions (e.g. second set of conditions) that allow development of said embryo to a three cerebral vesicles stage, wherein the conditions comprise a dynamic culture, an atmosphere comprising 15-40% oxygen; a medium comprising 15-75% serum.

Thus, according to specific embodiments, culturing of an embryo starting from the blastocyst stage is effected for about 3 days so at to obtain an embryo of a gastrulation stage, followed by culturing the gastrulation stage embryo for 2-3 days so as to obtain and embryo of a somitogenesis to early organogenesis stage, followed by culturing of the somitogenesis to early organogenesis stage embryo for about 3-4 days.

According to specific embodiments, culturing of an embryo starting from the blastocyst stage is effected for at least 5, at least 6, at least 7 or at least 8 days.

According to specific embodiments, culturing is effected from GD4 to GD12.

The blastocyst embryo of some embodiments of the invention may be obtained by isolating the blastocyst from a female rabbit. Methods of obtaining live undamaged rabbit blastocysts are known in the art and are disclosed for example in e.g. Bernd Pushel et.al 2010 cold spring harb protoc, 1, the contents of which are fully incorporated herein by reference.

According to other specific embodiment, the blastocyst stage embryo is obtained from a previously cultured embryo. Several such methods are known in the art and are disclosed in e.g. Bernd Pushel et.al 2010 cold spring harb protoc, 1, the contents of which are fully incorporated herein by reference, and in the Examples section which follows, and include culturing of cells following in-vitro fertilization or following zygote isolation until the implanting stage [e.g. in a static culture in a Continuous Single Culture Complete (CSCM) medium] and optionally removal of the zona pellucida. According to specific embodiments, the blastocyst embryo is further treated with pronase, as further described hereinabove, prior to culturing.

Hence, the method of some embodiments of the invention comprises in-vitro or ex-utero culturing of a rabbit embryo from E0 to the three cerebral vesicles stage, or any developmental stage therein-between.

According to specific embodiments, the method further comprises determining development of the embryo prior to, during and/or following the culturing. Methods of assessing development are well known in the art and are further described in details hereinabove and below.

Culturing conditions including e.g. type, media, pressure, oxygen concentrations and the like that can be used with specific embodiments of the rabbit embryo aspects are further described in details hereinabove and below.

However, according to specific embodiments of the rabbit embryo aspects, the serum comprises rabbit serum.

According to specific embodiments, the serum comprises rabbit serum and human serum.

In some embodiments, optionally, the medium further comprises knockout serum replacement (KSR) in addition to the rabbit serum and the human serum.

In some embodiments, optionally, the KSR partially replaces one of either the human serum, the rabbit serum or partially replaces a quantity of both.

According to specific embodiments, the serum comprises rabbit serum and human serum starting the latest when the rabbit embryo reaches a somitogenesis stage.

According to specific embodiments, the ratio between the rabbit serum and the human serum in the medium is between 1:1-5:1 (v/v).

According to specific embodiments, the ratio between the rabbit serum and the human serum in the medium is between 1:1-3:1 (v/v).

According to specific embodiments, the ratio between the rabbit serum and the human serum in the medium is about 2:1 (v/v).

According to specific embodiments, the ratio between the rabbit serum and the human serum in the medium is 2:1 (v/v).

According to specific embodiments of the rabbit embryo aspects, the rabbit embryo culture is maintained under a hyperbaric pressure starting the latest when embryo reaches a somitogenesis stage.

In order to control the pressure and oxygen level in the culture the present inventors have developed a novel fetal incubation system comprising a gas and pressure controller and a static and/or rotating incubator. Thus, according to specific embodiments, the culturing is effected using the fetal incubation system disclosed herein.

### Exemplary Fetal Incubation System

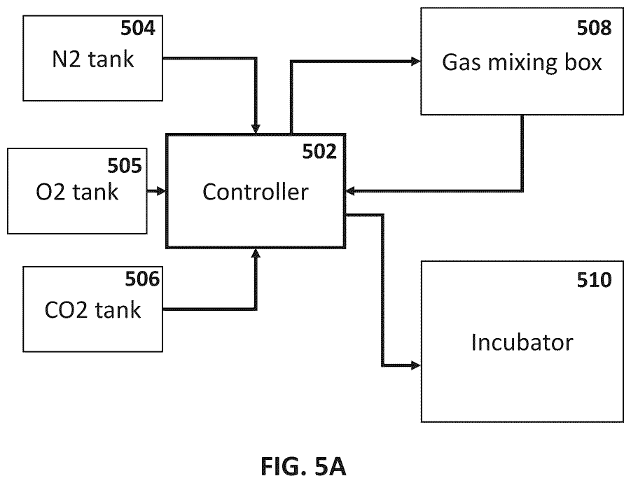

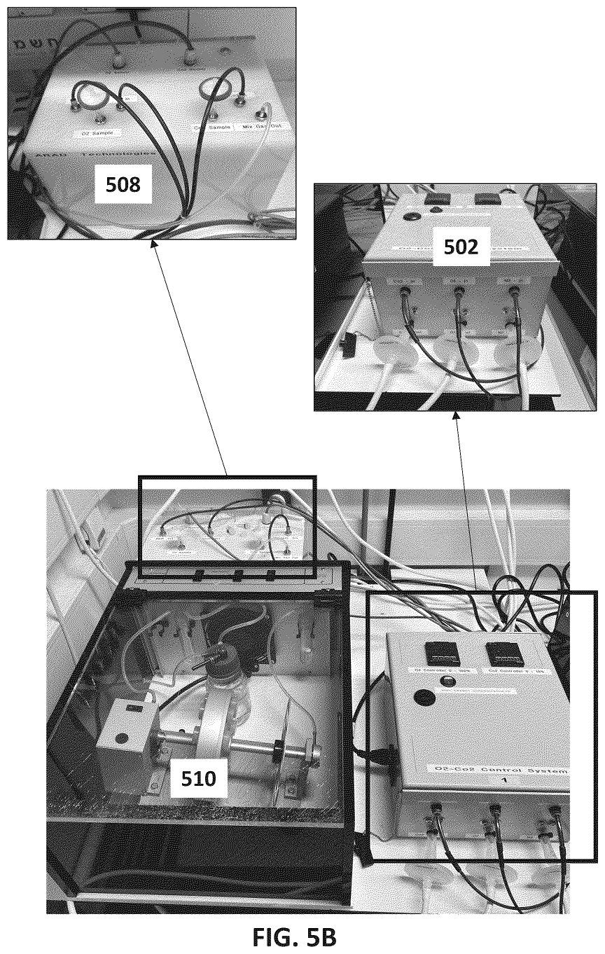

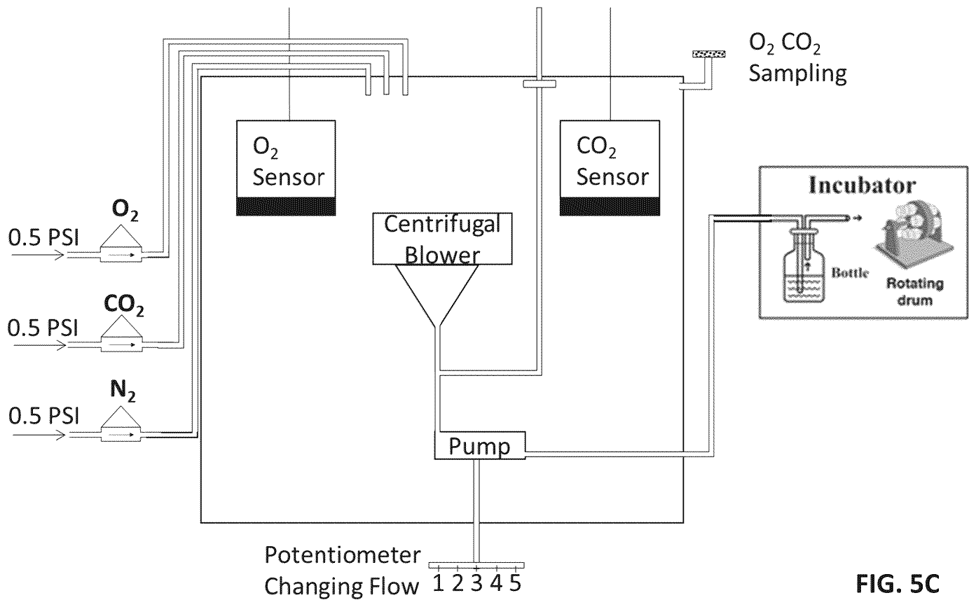

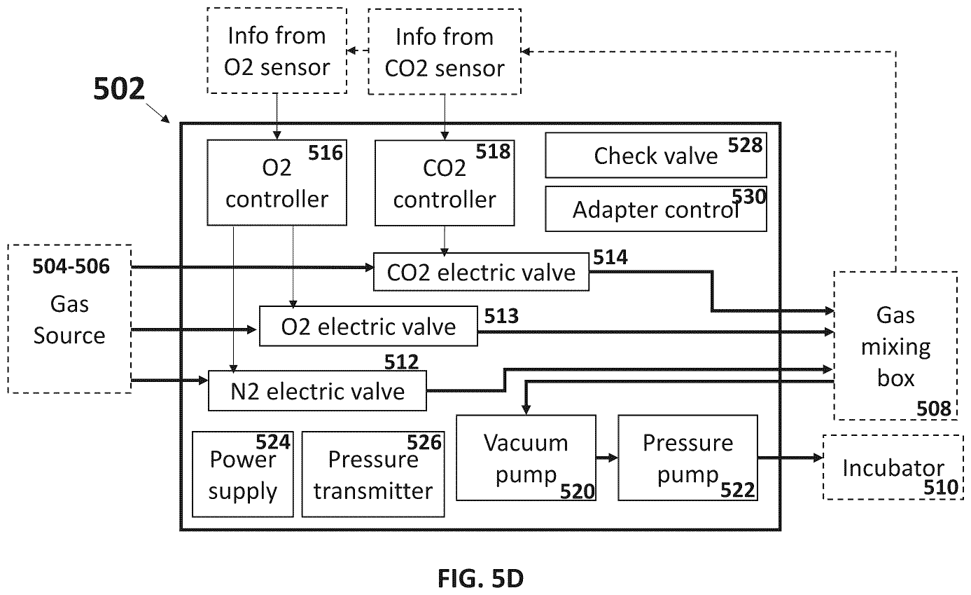

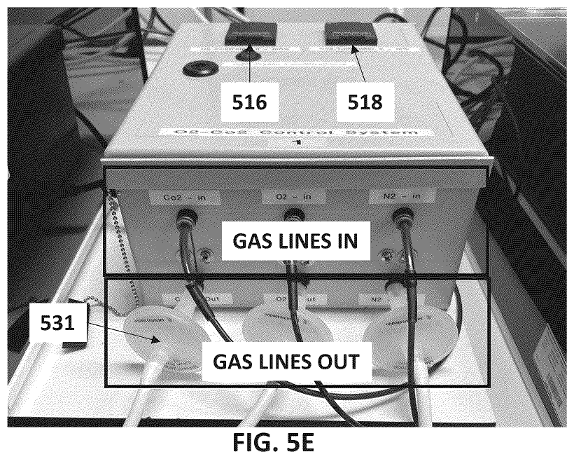

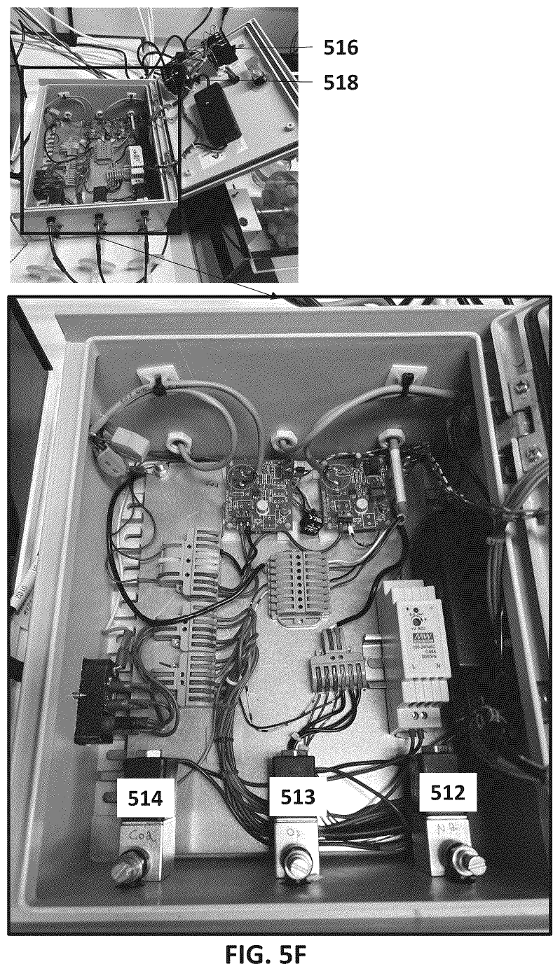

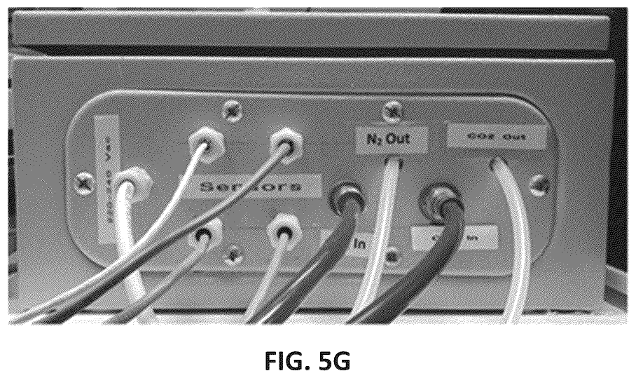

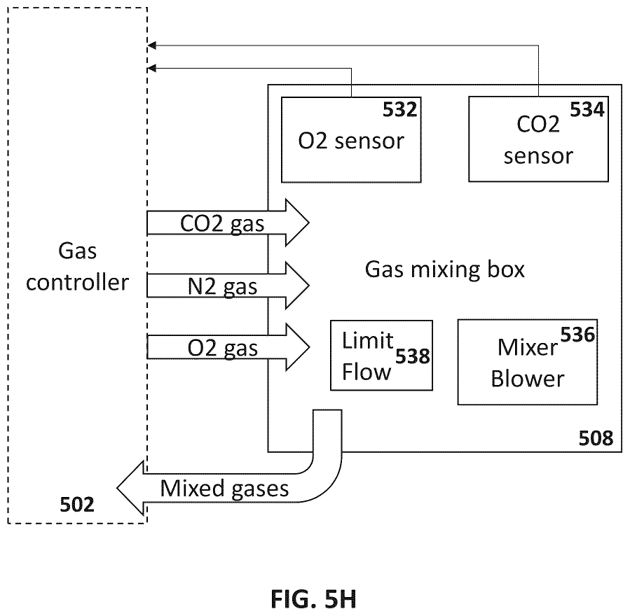

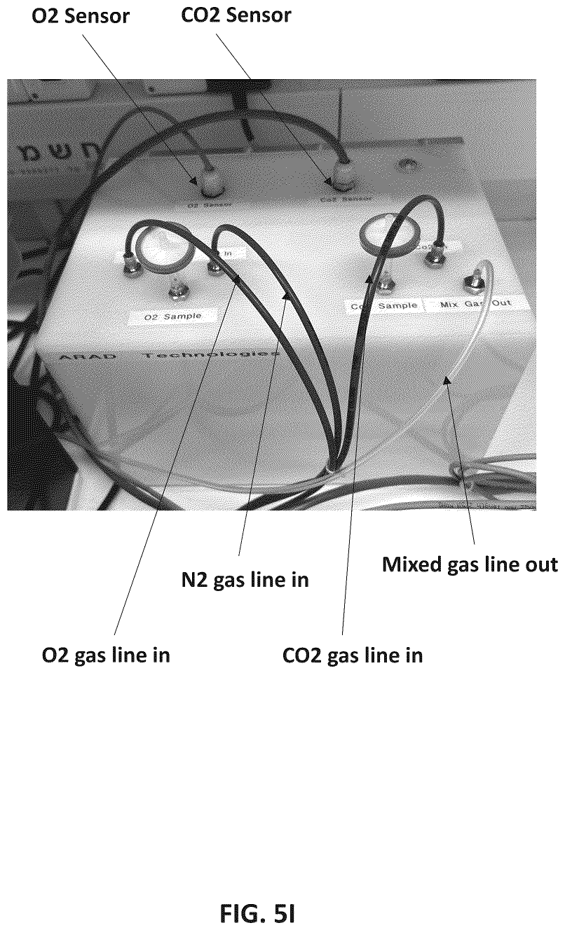

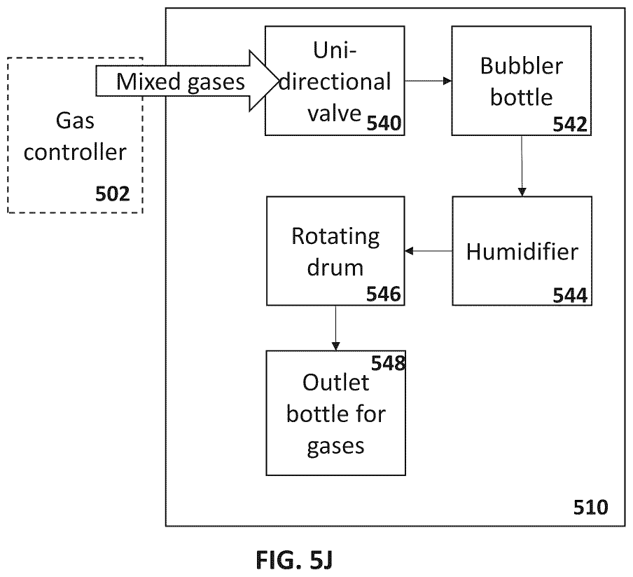

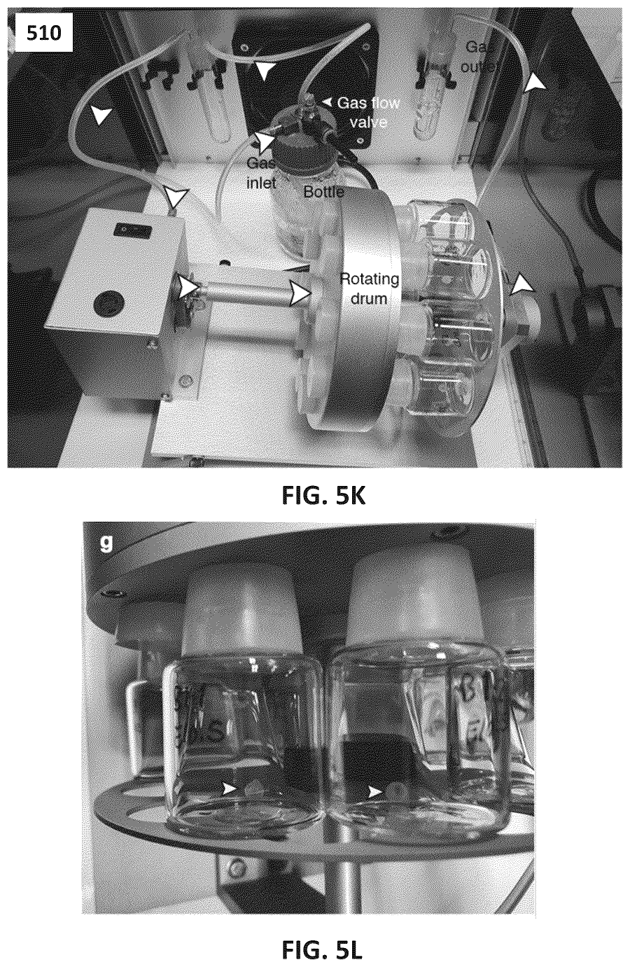

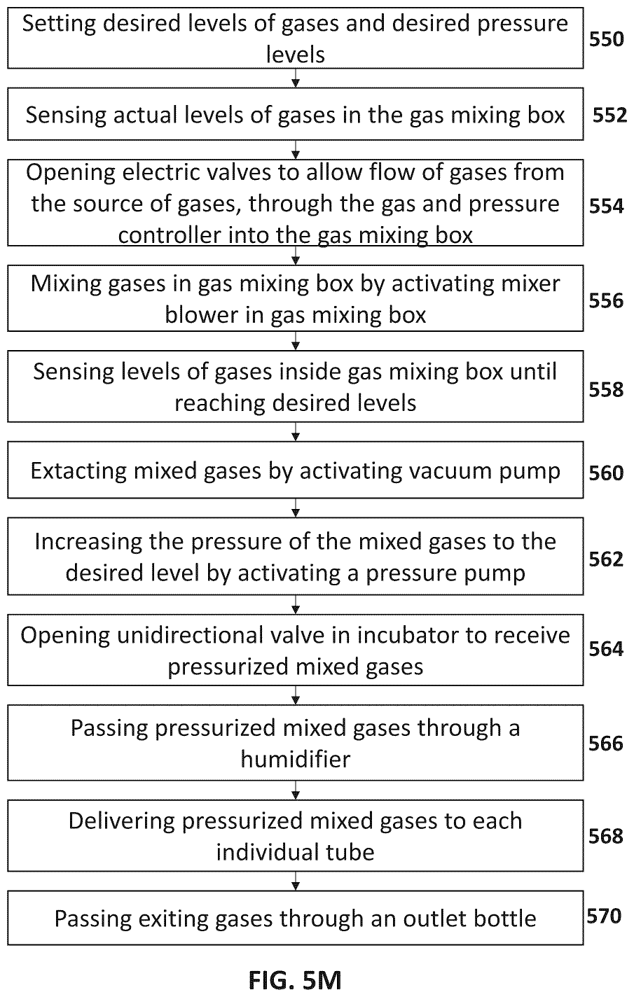

Referring now to FIG. 5A, showing a schematic representation of a fetal incubation system, according to some embodiments of the invention. In some embodiments, the system comprises a gas and pressure controller 502, one or more sources of gas 504, 505 and 506 (in FIG. 5A—Carbon dioxide (CO₂) 506, Oxygen (O₂) 505 and Nitrogen (N₂) 504 tanks are shown), a gas mixing box 508 and an incubator 510. In some embodiments, gases from the gas sources 504, 505, 506 are delivered into the gas and pressure controller 502, which delivers the gases into the gas mixing box 508. In some embodiments, once the mix of gases have reached the required concentrations, the gas is returned into the gas and pressure controller 502, which is then controlled-delivered into the incubator 510. In some embodiments, the incubator 510 optionally comprises an internal rotating incubator module configured to hold one or more vials in which the embryos are kept. In some embodiments, the mixed gases are delivered into the internal rotating incubator module, where the gases are equally delivered into each of the vials (see below for more information).

FIG. 5B shows an image of an exemplary fetal incubation system comprising the gas and pressure controller 502, the gas mixing box 508 and the incubator 510, according to some embodiments of the invention.

FIG. 5C shows a schematic general representation of an exemplary configuration of a principle of the electronic module for gas (gas and pressure controller 502 together with the gas mixing box 508) and pressure regulation. In some embodiments, N₂, O₂ and/or CO₂ enter into the system at a pressure of 0.5 psi and are mixed by a mixing centrifugal blower (see below). In some embodiments, gases are then optionally injected into a water bottle inside the incubator by a pump that allows control of the gas pressure, therefore allowing for hyperbaric conditions. In some embodiments, the system comprises one or more sampling ports (for example for O₂ and/or CO₂), which allow additional monitoring of the levels of the gases in the system.

### Exemplary Gas and Pressure Controller

Referring now to FIGS. 5D, showing a schematic representation of an exemplary gas and pressure controller 502, according to some embodiments of the invention. In some embodiments, as mentioned before one or more sources of gas 504, 505, 506 are connected to the gas and pressure controller 502. In some embodiments, each source of gas is connected to a dedicated electric valve 512, 513 and 514 in the gas and pressure controller 502. For example, gas from a source (for example a tank) of CO₂ 506 is connected to a dedicated CO₂ electric valve 514, and gas from a source (for example a tank) of N₂ 504 is connected to a dedicated N₂ electric valve 512, and gas from a source (for example a tank) of O₂ 505 is connected to a dedicated O₂ electric valve 513. In some embodiments, the gas and pressure controller 502 comprises dedicated ‘individual gas controllers 516/518’ for the manipulation and monitoring of the gases in the system (referred hereinafter as CO₂ controller 518 or O₂ controller 516—which are different from the main gas and pressure controller 502 of the fetal incubation system). For example, when the levels of CO₂ in the fetal incubation system are needed to be manipulated, a user accesses the CO₂ controller 518 to set up the required levels of CO₂ in the system, and for example when the levels of O₂ in the fetal incubation system are needed to be manipulated, a user accesses the O₂ controller 516 to set up the required levels of O₂ in the system by the addition or non-addition of N₂ gas into the system and/or by the addition or non-addition of O₂ gas into the system. It should be noted that in regular air there is about 21% oxygen, and when lower levels of oxygen are required inside the fetal incubation system, for example 5% or 10%, then nitrogen gas is inserted in order to reduce the levels of oxygen in the system. In some embodiments, when higher concentrations of oxygen are required, N₂ gases are stopped, and pure O₂ is provided, for example, to provide a 95%/5% O₂/CO₂ level inside the vials. In some embodiments, the system is configured to provide any combination of mixture of gases, for example from a mixture of gases that comprises 0% of O₂ to providing 100% of O₂; or for example any combination of O₂/CO₂ ratios, for example from about 1%/99% ratio to a 99%/1% ratio. In some embodiments, the gas and pressure controller 502, provides and ensures gases with a margin of error of about 0.2% for any of the gases provided to the fetal incubation system. In some embodiments, the margin of error is from about 0.1% to about 1% for CO₂ gases, from about 0.1% to about 2% for O₂ gases and from about 0.1% to about 2% for N₂ gases. In some embodiments, margins of error are not above 2% for CO₂. In some embodiments, margins of error are not above 5% for N₂. In some embodiments, margins of error are not above 5% for O₂. In some embodiments, each specific gas controller 516/518 controls the opening and closing of the specific electric valves 512, 513 and 514, according to the needs. In some embodiments, the needs, which are set by the user using the individual gas controllers 516/518, are monitored by dedicated sensors in the gas mixing box 508 (see below information about gas mixing box 508). Therefore, in some embodiments, information from the gas sensors in the gas mixing box 508 are delivered to the dedicated gas controllers 516/518. In some embodiments, the dedicated gas controllers 516/518 utilize the information from the gas sensors in the gas mixing box 508 to either open or close the specific electric valves 512, 513, 514, again according to the predetermined needs set by the user. In some embodiments, the gas and pressure controller 502 comprises a vacuum pump 520 and a pressure pump 522. In some embodiments, after the gases have been mixed in the gas mixing box 508 (see below information about gas mixing box 508), a vacuum pump 520 is activated to force out the mixed gases from the gas mixing box 508. In some embodiments, the gases are then accumulated in a pressure pump 522 until a predetermined level of pressure is reached before it is delivered into the incubator 510. In some embodiments, the pressure pump is configured to provide the mixed gases at a pressure of from about 1 psi to about 6 psi, optionally of from about 0.5 psi to about 8 psi, optionally from about 0.05 psi to about 12 psi. Preferably at 6.5 psi, when needed. In some embodiments, the user manually sets the pressure levels on which the pressure pump 522 will delivered the mixed gases. In some embodiments, the gas and pressure controller 502 comprises a power supply unit 524 configured to provide the dedicated power to the different parts of the gas and pressure controller 502. In some embodiments, the gas and pressure controller 502 optionally comprises a pressure transmitter 526 configured to monitor the pressure in the system/gas and pressure controller 502. In some embodiments, the gas and pressure controller 502 optionally comprises a check valve 528 configured to ensure that mixed gases exiting the gas and pressure controller 502 do not return (flow back) into the gas and pressure controller 502. In some embodiments, the gas and pressure controller 502 optionally comprises an adapter control for gases 530 configured to control the flow rate in the system. In some embodiments, the gas and pressure controller 502 comprises one or more filters—shown in FIG. 5E (531)—(for example 1μ filters) mounted on the tubes flowing gases for ensuring purity of the gases and potentially avoid contaminations.

FIGS. 5E, 5F and 5G show different images of an exemplary gas and pressure controller 502, according to some embodiments of the invention. Specifically, FIG. 5E shows a perspective view of the gas and pressure controller 502, showing the gas lines that go into the gas and pressure controller 502, and the gas lines that go out from the gas and pressure controller 502. Additionally, gas controllers 516/518 and optional filters 531 are shown. FIG. 5F shows the internal arrangement of the gas and pressure controller 502, showing exemplary electric valves 512, 513, 514, according to some embodiments of the invention. FIG. 5G shows an exemplary gas and pressure controller 502 that is configured to monitor and deliver only CO₂ and/or N₂, according to some embodiments of the invention.

Exemplary gas mixing box Referring now to FIG. 5H, showing a schematic representation of an exemplary gas mixing box 508, according to some embodiments of the invention. In some embodiments, the gas mixing box 508 is used to ensure complete and uniform mixing of the different gases that are required to provide the necessary environment in the vials in the incubator. In some embodiments, the gas mixing box 508 comprises an internal volume of from about 250,000 cm³ to about 260,000 cm³. In some embodiments, different sizes may be used to provide different quantities of mixed gases as necessary. In some embodiments, the gas mixing box 508 is made of plastic, for example Perspex. In some embodiments, the gas mixing box 508 is made of a material other than plastic. In some embodiments, the gas mixing box 508 comprises dedicated gas sensors 532/534 configured to monitor the content percentage of those gases inside the gas mixing box 508. Following the previous description, in FIG. 5H two sensors are shown, an O₂ sensor 532 and a CO₂ sensor 534. In some embodiments, the sensors comprise a sensitivity for accuracy of from about 95% accuracy to about a 100% accuracy. In some embodiments, the gas mixing box 508 comprises a mixer blower 536 configured to thoroughly mix the gases coming from the gas and pressure controller 502. In some embodiments, once the levels of the gases detected inside the gas mixing box 508 by the sensors 532/534 arrive at the desired level, the mixed gases are sucked away by the vacuum pump 520 in the gas and pressure controller 502. In some embodiments, the gas and pressure controller 502 optionally comprises a limit flow 538 configured to maintain a uniform flow rate in the system, therefore potentially avoiding the possibility of changes in the flow rate. In some embodiments, the gas mixing box 508 comprises one or more filters—not shown—(for example 1μ filters) mounted on the tubes flowing gases for ensuring purity of the gases and potentially avoid contaminations.

FIG. 5I shows an image of an exemplary gas mixing box 508, according to some embodiments of the invention.

### Exemplary Incubator

In some embodiments, the incubator is a static incubator. In some embodiments, the incubator is a rotating incubator. In some embodiments, the incubator is a static incubator comprising a rotating module inside of it. In some embodiments, the static incubator comprises one or more temperature modulators configured to preserve the temperature inside the static incubator, including the rotating module allocated inside of it. In some embodiments, the temperatures inside the incubator are modulated to be from 4° C. to about 60° C. In some embodiments, the incubator is, for example, a “precision” incubator system (BTC01 model with gas bubbler kit—by B.T.C. Engineering,—Cullum Starr Precision Engineering Ltd—UK).

Referring now to FIG. 5J, showing a schematic representation of an exemplary incubator 510, according to some embodiments of the invention. In some embodiments, mixed gases are delivered from the gas and pressure controller 502 into the incubator 510. In some embodiments, the incubator comprises a unidirectional valve 540 connected to a tube, from which the mixed gases are delivered from the gas and pressure controller 502. In some embodiments, the unidirectional valve 540 is a manual unidirectional valve, which is opened and closed manually by a user. In some embodiments, the unidirectional valve 540 is an automatic unidirectional valve controlled by a master controller (see below). In some embodiments, the mixed gases are optionally delivered into a bubbler bottle 542. In some embodiments, the bubbler bottle 542 is partially filled with a liquid. In some embodiments, the liquid is water. In some embodiments, the gases are delivered into the liquid in the bubbler bottle 542, thereby creating bubbles in the liquid. In some embodiments, the bubbler bottle 542 allows a user to see that the system is delivering mixed gases by visually assessing: 1. If there are bubbles; and 2. The rate of creation of bubbles. In some embodiments, additionally, bubbler bottle 542 works as a humidifier for the gases (see below). In some embodiments, the bubbler bottle 542 provides a safeguard from pressure coming from the delivered mixed gases into the incubator 510, for example, in case of a malfunction if mixed gases are delivered at higher pressure than the desired one, the extra pressure will be contained and dissipated in the bubbler bottle 542. In some embodiments, the mixed gases are optionally then delivered into an additional humidifier 544. In some embodiments, the inventors have discovered that in certain cases dry mixed gases could be harmful to the samples in the incubator, therefore, the addition of the humidifier 544 overcomes this issue. In some embodiments, the additional humidifier reduces excess humidity from the gases coming from bubbler bottle 542. In some embodiments, from the humidifier 544, the mixed gases are delivered into the rotating drum 546 of the rotation module, which comprises all containers (vials) comprising the samples located in the incubator. In some embodiments, the rotating drum 546 is configured to deliver mixed gases equally between the containers, optionally while rotating the samples. In some embodiments, the containers (vials) in the rotating drum 546 comprise the medium necessary for the growth and/or maintenance of the embryos. In some embodiments, the delivery of the gases is provided into the containers (vials) and absorbed/used via the medium. It should be noted that, in some embodiments, since the embryos are left in suspension in the medium, continuous delivery of new medium with already mixed gases is problematic, because these types of mechanisms require old/used medium to be extracted from the vial while inserting new medium with new mixed gases in it, which can increase the chance of losing the embryos during the exchange. Therefore, the provision of new gases is performed by delivery mixed gases into the vials without the need to change the medium for it. In some embodiments, independently of the need of providing continuous replacement of gases, medium can be changed by taking out each vial and carefully changing the medium according to known techniques. In some embodiments, the rotating drum 546 is configured to be positioned in an angle, which allows the vials to have an angle with respect to the base on which the whole rotating module is standing. In some embodiments, the angle of from about 0 degrees (no angle—vials are kept on their side as shown for example in FIG. 5K) to about 45 degrees. In some embodiments, the angle is provided so a top of a vial is always in an upper position in relation to a bottom part of the vial. In some embodiments, the rotation of the rotating drum 546 is independent from the action of delivering mixed gases into the vials. In some embodiments, the rotating drum is configured to rotate at velocities of from about 1 rpm to about 100 rpm. In some embodiments, exiting gases from the rotating drum 546 are delivered to an outlet bottle 548 for gases. In some embodiments, the outlet bottle 548 acts as a pressure buffer for the system, which helps keeping a constant pressure throughout the system. In some embodiments, the incubator 510 comprises darkened walls, which allow keeping the samples in the dark. In some embodiments, the incubator 510 optionally comprises one or more cameras for monitoring the samples, for examples regular video cameras, IR cameras, night vision cameras, etc. In some embodiments, the incubator 510 comprises one or more heaters configured to keep the samples at a certain temperature. In some embodiments, the incubator 510 comprises one or more light sources, for example, white light, IR light, UV light and/or black light.

FIG. 5K shows an image of an exemplary rotating incubator module 510, according to some embodiments of the invention. FIG. 5L shows an image of exemplary containers (vials or bottles) having samples, according to some embodiments of the invention.

### Exemplary Automated Fetal Incubation System

In some embodiments, the fetal incubation system as disclosed above, is connected to a master controller configured to perform automated actions according to predetermined protocols provided by a user. For example, a user programs the master controller to perform changes in the incubation chambers over a certain period of time. In some embodiments, the system will comprise electric valves overall the system, which will be activated/deactivated according to the programed protocols. In some embodiments, a potential advantage of utilizing automated systems is that it reduces the chances of human errors during the developments of the embryos. In some embodiments, optionally, the master controller provides periodic updates to a user to a PC or a mobile electronic device.

### Exemplary General Information Related to the System

In general, a number of culture techniques have been proposed over the years since the 1930s by culturing the embryos in conventional static conditions, in rotating bottles on a drum (referred to as “roller culture systems”) or on circulator platforms. These platforms remain highly inefficient for embryos survival and are limited to short periods of time, as the embryos begin to display developmental anomalies as early as 24 hours after culture initiation. Thus, stable and efficient protocols for extended culturing of pre-gastrulating mouse embryos all the way until advanced organogenesis stages were developed and are disclosed herein. In some embodiments, some of cell culture supplements or biomechanical principles newly established in stem cell research, were tested to assess if they could be helpful for keeping embryos alive (e.g. hyperbaric chambers, synthetic sera). In some embodiments, the “roller culture system” on a drum is used and it is integrated with a customized and in house developed electronic gas regulation module 502 that allowed precise control not only of N₂, O₂ and CO₂ levels with high sensitivity, but also allowed controlling the atmospheric pressure. In some embodiments, sequential increases in the oxygen levels every 24 hours, starting from 5% O₂ at E7.5, 13% at E8.5, 18% at E9.5, and ending with 21% O₂ at E10.5 were applied and were found to be most optimal for the robust outcome reported herein. Additionally, when necessary, an increase in oxygen levels reached 95%. In addition, in some embodiments, maintaining a hyperbaric pressure of about 6.5 psi was found also critical for normal and efficient development of the embryos.

In some embodiments, the samples are kept in a static incubator. In some embodiments, the samples are kept in a dynamic incubator, for example a rotating incubator. In some embodiments, the samples are kept first in a static incubator and then moved to a dynamic incubator, or vice versa. In some embodiments, the samples are kept in a static incubator comprising, for example a rotating incubator inside of it. In some embodiments, when kept in a dynamic incubator the samples are kept in rotating bottles on a drum (referred to as “roller culture systems”) or on circulator platforms.

In some embodiments, for cultures starting at E7.5 or later stages, the embryos are kept on the rotating bottles culture unit inside a “precision” incubator system (For example the BTC01 model with gas bubbler kit—by B.T.C. Engineering,—Cullum Starr Precision Engineering Ltd—UK) during all the time of culture. In some embodiments, a ‘rotator’ culture method which provides continuous flow of oxygenating gas to cultures in rotating bottles was used and disclosed herein elsewhere (for example BTC Rotating Bottle Culture Unit BTC02 model by B.T.C. Engineering,—Cullum Starr Precision Engineering Ltd—UK). In some embodiments, the culture bottles (Glass Bottles (Small) BTC 03 and Glass Bottles (Large) BTC 04) are plugged into the hollow rotating drum. In some embodiments, gas flows along the axis and is distributed to the culture bottles by a baffle plate within the drum. In some embodiments, the system maintains a stable pH, when compared to other systems with sealed culture bottles. In some embodiments, the rotator is supplied complete with gas filter, bubbler and leads by the manufacturer. In some embodiments, the BTC Precision Incubator uses a thyristor-controlled heater and high flow-rate fan to give a highly stable and uniform temperature throughout the easily accessible working volume. In some embodiments, the incubator has a working volume 370×350×200 mm high which is accessed through a hinged top. In some embodiments, the heater element is rated at 750 Watts. In some embodiments, Bung (Hole) BTC 06 is used to seal the bottles and Bung (Solid) BTC 07 is used to seal the drum (B.T.C. Engineering,—Cullum Starr Precision Engineering Ltd—UK).

In some embodiments, in order to achieve constant O₂ and CO₂ levels in the culture medium throughout the incubation period, the incubator module is linked to the gas and pressure control unit 502 (model #-HannaLab1; assembled and sold by Arad Technologies LTD, Ashdod, Israel). In some embodiments, carbon dioxide and oxygen concentration are regulated by specific controllers located on the gas and pressure control unit 502. In some embodiments, a pressure controller allows control of the gas pressure between 5 to 10 psi (positive pressure over ambient external atmospheric pressure). In some embodiments, nitrogen, O₂ and/or CO₂ are then injected into the gas mixer box at pressure of 6.5 psi which was found as the optimal level. In some embodiments, the mixing of the gases in the gas box is homogeneous and mixed by a centrifugal mixer blower. In some embodiments, the gases are injected into the incubator by a pump that builds pressure and sufficiency according to the count of air bubbles created in a water bottle, which is under the control of a one-way flow meter. In some embodiments, the bubble rate (which indicates the speed of gas flowing into the bottles) is be adjusted as needed by the user. In some embodiments, gas flows through the inlet into the water bottle, and the speed of gas flowing into the bottle is controlled with a valve. In some embodiments, humidified gas circulates to a glass test tube and then to the inside of the bottles in the rotating drum. In some embodiments, gas flow speed is monitored by the rate of bubbles created inside an outlet water-filled test tube. In some embodiments, the bottles with the samples are placed on the rotating bottle culture system, rotating at 30 revolutions per minute at 37° C., and continuously gassed with an atmosphere of, for example 5% O₂, 5% CO₂ at 6.5 pounds per square inch (psi), or for example with a gas mixture of 13% O₂, 5% CO₂, or for example in a gas atmosphere of 18% O₂ and 5% CO₂, or for example with a gas supply of 21% O₂ and 5% CO₂. In some embodiments, for media exchange, culture media is pre-heated for at least an hour by placing it inside a glass bottle on the rotating culture with an adequate gas atmosphere depending on the stage of the cultured embryos.

### Exemplary Methods Related to the Fetal Incubation System

Referring now to FIG. 5M, showing a flowchart of exemplary methods related to the fetal incubation system, according to some embodiments of the invention. In some embodiments, the user sets the desired levels of gas and pressure inside the incubator 550. For example 5% CO₂ and 10% O₂ at a pressure of 6.1 psi. In some embodiments, the gas and pressure controller activates the sensors in the gas mixing box to receive information about the actual levels of the gases in the gas mixing box 552. In some embodiments, the electric valves are opened to allow flow of gases from the gas sources, through the gas and pressure controller into the gas mixing box 554. In some embodiments, the gases are mixed inside the gas mixing box by activating a mixer blower 556. In some embodiments, the sensors in the gas mixing box are activated to monitor the levels of the gases inside until they reach the desired levels 558. In some embodiments, the mixed gases are extracted from the gas mixing box by activating a vacuum pump 560. In some embodiments, pressure of the extracted mixed gases are increased to a desired level by activating a pressure pump 562. In some embodiments, a unidirectional valve inside the incubator is opened to allow the pressurized mixed gases to flow into the incubator 564. In some embodiments, the pressurized mixed gases are passed through a humidifier 566. In some embodiments, the humidified pressurized mixed gases are delivered to the individual tubes containing the samples 568. In some embodiments, exiting gases are passed through an outlet tube before exiting the incubator 570.

Establishment of methods and fetal incubation systems for growing normal mouse and rabbit embryos ex utero as described herein may be further combined with e.g. genetic modification, chemical screens, tissue manipulation and microscopy methods and may constitute a powerful tool in basic research e.g. as a framework to investigate the emergence of cellular diversity, cell fate decisions and how tissues and organs emerge from a single totipotent cell; as well as a source of cells, tissue and organs for transplantation, generation of chimeric embryos, testing the effect of drugs on embryonic development (e.g. teratogenic effect) etc. Such methods are known in the art and are also described in the Examples section which follow.

Thus, according to specific embodiments, the method comprises manipulating the embryo prior to, during or following the culturing.

According to specific embodiments, manipulating comprises introducing into the embryo a gene of interest.

According to specific embodiments, manipulating comprises introducing into the embryo a polynucleotide of interest.

According to specific embodiments, manipulating comprises introducing into the embryo a genome editing or RNA silencing agent.

According to specific embodiments, the manipulating comprises producing an embryo incompatible with life. Thus, for example, the manipulation may comprise knocking a selected gene to selectively perturb a certain organ, thus making the embryo with limited developmental potential and not being able to sustain viability, e.g. headless (e.g. deletion of Mesp1 or NKX2-5) or heartless (e.g. deletion of Lim1), as further described in the Examples section which follows.

Thus, according to specific embodiments, the manipulating comprises introducing into the embryo a polynucleotide rendering an embryo incompatible with life.

Methods of designing, expressing and introducing a polynucleotide of interest such that it will be expressed in a cell of interest are well known in the art and thus need.

According to specific embodiments, the introducing is effected by electroporation and viral (e.g. lentiviral) infection. Non-limiting Examples of such methods and conditions that can be used with specific embodiments of the invention are further described in the Examples section which follows.

According to specific embodiments, the electroporation conditions comprise: Two poring pulses applied at 10-100V with a duration of 2-30 milliseconds (ms) each, a pulse interval of 45-450 ms and a decay rate of 5-15%, followed by five transfer pulses applied at 15-50 V for 20-60 ms each with an interval of 45-450 ms between pulses and a voltage decay of 30-50% a.

According to specific embodiments manipulating comprises microinjecting cells into said embryo to thereby obtain a chimeric embryo.

As used herein, the phrase “chimeric embryo” refers to an animal comprising cells of at least two genetically distinct individuals.

It is noted that the chimeric embryo can be composed of cells of two different individuals belonging to two different species, or to the same species.

According to some embodiments of the invention, the cells are allogeneic to the mouse embryo.

As used herein, the term “alloegeneic” refers to at least two genetically different mice.

According to some embodiments of the invention, the cells are xenogeneic to the mouse embryo.

As used herein, the term “xenogeneic” refers to at least two individuals of different species.

Hence, according to specific embodiments, the cells are mammalian cells.

According to specific embodiments, the cells are human cells.

According to specific embodiments, the cells are stem cells [for example, but not limited to, embryonic stem cells, mesenchymal stem cells, neural stem cells, hematopoietic stem cells, induced pluripotent stem cells (iPS)].

According to some embodiments of the invention, introducing the cells is performed ex vivo via direct injection or aggregation with the developing embryo.

According to some embodiments of the invention, the cells (e.g. ESC/iPSCs) are injected at the 2 cell embryo stage to generate “all ESC/iPSC” chimeras, as further described in Example 6 of the Examples section which follows.

According to specific embodiments, the manipulating comprises introducing into the embryo a drug of interest.

According to specific embodiments, the methods further comprise determining an effect of the manipulating on development of the embryo.

According to specific embodiments, the methods further comprise isolating a cell, tissue or organ from the embryo following the culturing.

Non-limiting examples of such cells include stem cells [for example, but not limited to, embryonic stem cells, mesenchymal stem cells, neural stem cells, hematopoietic stem cells], blood cells, liver cells, insulin secreting pancreatic beta cells, muscle cells, lung epithelial cells, endothelial cells, glial cells.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion. Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8^{th} Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods for Examples 1-7

Data reporting—No statistical methods were used to predetermine sample size. The experiments were not randomized and the investigators were not blinded to allocation during experiments and outcome assessment.

Animals—Female 5-8-week old ICR, C57BL/6 or BDF1 mice were mated with matched BDF1 male studs (Harlan). For experiments using transgenic reporter lines mTmG (Gt(ROSA)26Sor^{tm4(ACTB-tdTomato,-EGFP)Luo}) (Jackson #007576) females were mated with either Wnt1-Cre (Jackson #022137) or Isl1-Cre (Jackson #024242) males. For live imaging and post implantation grafting experiments, Ail4(RCL-tdT)-D (B6.Cg-Gt(ROSA)26Sor^{(CAG-tdTomato)Hze}) (Jackson #007914) mice were crossed with Stra8-iCre, F1 males were mated with ICR females, and Td-Tomato⁺ embryos were selected. For imprinting experiments, Dlk1-Dio3 IG-DMR-Snrpn-GFP (Jackson #030539) males were mated with ICR or C57BL/6 females. Insemination was verified the next morning by the presence of a copulatory plug, and this day was defined as E0.5 days post coitum (d.p.c). All animal experiments were performed according to the Animal Protection Guidelines of Weizmann Institute of Science, and approved by relevant Weizmann Institute IACUC. All mice were housed in a standard 12-hours light/12-hours dark cycle conditions in a specialized and certified animal facility.

Ex utero whole embryo roller culture and gas regulation module—For cultures starting at E7.5 or later stages, the embryos were kept on a rotating bottles culture unit inside a “precision” incubator system (BTC01 model with gas bubbler kit—by B.T.C. Engineering,—Cullum Starr Precision Engineering Ltd—UK) during all the time of culture. A ‘rotator’ culture method which provides continuous flow of oxygenating gas to cultures in rotating bottles (BTC Rotating Bottle Culture Unit BTC02 model by B.T.C. Engineering,—Cullum Starr Precision Engineering Ltd—UK) was utilized. Culture bottles [Glass Bottles (Small) BTC 03 and Glass Bottles (Large) BTC 04] were plugged into the hollow rotating drum. In this system, oxygenating gas flows along the axis and is distributed to the culture bottles by a baffle plate within the drum. The system maintains a more stable pH than systems with sealed culture bottles. The rotator was supplied complete with gas filter, bubbler and leads by the manufacturer. The BTC Precision Incubator uses a thyristor-controlled heater and high flow-rate fan to give a highly stable and uniform temperature throughout the easily accessible working volume. The incubator has a working volume 370×350×200 mm high which is accessed through the hinged Perspex top. The heater element is rated at 750 Watts. Bung (Hole) BTC 06 was used to seal the bottles and Bung (Solid) BTC 07 was used to seal the drum (B.T.C. Engineering,—Cullum Starr Precision Engineering Ltd—UK). In some embodiments, the incubator is covered by a black piece of cloth to induce darkness for the majority of time during which the embryos are growing which, in some embodiments, is critical for the success of the experiment.

In order to achieve constant O₂ and CO₂ levels in the culture medium throughout the incubation period, the incubator module was linked to an in-house designed and customized gas and pressure control unit (model #-HannaLab1; assembled and sold by Arad Technologies LTD, Ashdod, Israel). In this designed system, carbon dioxide and oxygen concentration are regulated by specific controllers located inside the regulation module. A pressure transmitter allows control of the gas pressure between 5 to 10 psi (positive pressure over ambient external atmospheric pressure). N₂ and CO₂ are then injected into the gas mixer box at pressure of 6.5 psi which was found as the optimal level. The mixing of the gases in the gas box is homogeneous and mixed by a centrifugal blower. The gases are injected into the incubator by a pump that builds pressure and sufficiency according to the count of air bubbles created in a water bottle, which is under the control of a one-way flow meter. The bubble rate (which indicates the speed of gas flowing into the bottles) can be adjusted as needed by the user. The main components of the system are the following: Oxygen and CO₂ controller, pressure pump, vacuum pump, oxygen and CO₂ sensors, power supply, check valve, mix gas box, pressure transmitter, limit flow, adapter control for gases, 1 μm filters, centrifugal blower (see FIGS. 5A-L). The gas control unit established here can be purchased from Arad Technologies Ltd., Ashdod, Israel. Gas flows through the inlet into the water bottle, and the speed of gas flowing into the bottle can be controlled with a valve (yellow arrowhead). Humidified gas circulates to a glass test tube and then to the inside of the bottles in the rotating drum; gas flow speed can be monitored by the rate of bubbles created inside an outlet water-filled test tube.

Isolation of human umbilical cord blood serum and adult blood serum—Umbilical blood was collected from umbilical cords of healthy pregnant women over the age of 18 and under 40, who gave their consent and were scheduled for caesarian section delivery by their obstetrician following a prenatal clinic visit (approved by a Rambam Medical Center Helsinki committee). The source of each collection underwent full anonymization and was not identified by name or other designation, and the extracted serum was only used as described herein. Women who gave vaginal birth as well as women with any chronic illness or active medical conditions, including gestational diabetes or hypertension, were excluded. On the day of scheduled caesarian delivery and in order to ensure fresh isolation and processing of serum, a team stood by for cord blood collection and serum extraction. Immediately upon delivery of the infant, the umbilical cord was double clamped 5-7 cm from the umbilicus and transected between the clamps. Blood was collected only after the infant was removed from the field of surgery and umbilical blood was drawn for clinical tests as needed. In order to avoid any traces of hemolysis, blood was manually drawn by the obstetrician surgeon, using a large bore 14-gauge needle and a 50 ml syringe, directly from the umbilical vein while the placenta remained in situ. This was done to avoid any coagulation of blood before collection which could lead to traumatic hemolysis, and also to take advantage of the enhanced blood flow generated by uterine contraction. Next, the collected blood was freshly and quickly distributed to 5 ml pro-coagulant sterile test tubes (Greiner Bio-One, Z Serum Sep Clot Activator, #456005) and cooled to 4° C. for 15 minutes, to allow full coagulation. Following, coagulated test tubes were centrifuged at 2500G for 10 minutes in a cooled 4° C. centrifuge. Any tube that showed signs of hemolysis (such as pinkish-red colored serum) was discarded. The separated serum (yellowish colored) was collected using a pipette and filtered through a 0.22 μM filter (Nalgene, Ref #565-0020) and then inactivated in 55° C. bath for 45 minutes. The inactivated serum was next distributed to aliquots and placed in a −80° C. freezer for storage for up to six months. Shipping temperature was kept at −70° C. using dry ice and any thawed serum was refrozen once. Human adult blood serum was collected from healthy adults and freshly prepared with the same protocol described for umbilical cord blood serum.

Ex utero embryo culture media (EUCM)—EUCM, also referred to herein as “EUCM1” consisted of 25% DMEM (GIBCO 11880; includes 1 mg/mL D-glucose and sodium pyruvate, without phenol red and without L-glutamine) supplemented with 1× Glutamax (GIBCO, 35050061), 100 units/ml penicillin/100 μg/ml streptomycin (Biological industries; 030311B) and 2 mM HEPES (GIBCO, 15630056), plus 50% Rat Serum (RS) (Rat whole embryo culture serum, ENVIGO Bioproducts B-4520) and 25% Human Umbilical Cord Blood Serum (HCS) or human Adult Serum. DMEM (GIBCO 11880) supplemented with Glutamax, Pen/Strep and HEPES was stored at 4° C. in aliquots and used within 2 months. Rat serum was stored at −80° C. and heat inactivated at 56° C. for half an hour and filtered through a 0.22 μm PVDF filter (Millipore; SLGV033RS) prior to use. HCS was collected at Rambam Medical Center in Haifa, Israel, and stored as heat inactivated and filtered aliquots at −80° C. as described hereinabove. HCS was freshly thawed and used immediately before experimentation. In some experiments, HCS was replaced by Human Adult Blood Serum (HBS or HAS). HBS was freshly collected and stored as heat inactivated and filtered aliquots at −80° C. Rat serum, HCS and HBS can be thawed/frozen once. When indicated, the medium was supplemented with extra D-glucose (J.T. Baker) and sodium pyruvate (Sigma-Aldrich, cat. no. P4562). Advanced DMEM F12 (Invitrogen) or CMRL media give similar results in EUCM when they replace DMEM (Invitrogen).

During the process of media optimizations, to reach a meaningful conclusions regarding each sera or tissue culture supplement, at least 3 different lots (batches) of reagent form the same vendor were used. The following supplements were tested: KSR (KnockOut™ Serum Replacement, GIBCO 10828010), HPLM (Human plasma-like media) kindly provided by Jason Cantor¹², N-2 Supplement (100×) (GIBCO, 17502048), B-27™ Supplement (50×) (GIBCO, 17504044).

EUCM2 consisted of 80% CMRL (Gibco 11530037), supplemented with 1× Glutamax (GIBCO, 35050061), 100 units/ml penicillin/100 μg/ml streptomycin (Biological industries; 030311B) and 1 mM sodium pyruvate (Sigma-Aldrich, cat. no. P4562) plus 20% FBS.

EUCM3 consisted of CMRL, (Gibco 11530037), supplemented with 1× Glutamax (GIBCO, 35050061), 100 units/ml penicillin/100 μg/ml streptomycin (Biological industries; 030311B) and 1 mM sodium pyruvate (Sigma-Aldrich, cat. no. P4562) plus 30% HAS.

EUCM4 consisted of CMRL, (Gibco 11530037), supplemented with 1× Glutamax (GIBCO, 35050061), 100 units/ml penicillin/100 μg/ml streptomycin (Biological industries; 030311B) and 1 mM sodium pyruvate (Sigma-Aldrich, cat. no. P4562) plus 40% HAS.

In some of the experiments, the EUCM2/3/4 media were further supplemented with non-essential amino acids (NEAA) 1×, 4 mg/mL D-Glucose, ITS-X 1× (Gibco 51500056), 3 nM Beta-Estradiol (Sigma-Aldrich, cat. no. E8875), 20 ng/ml Progesterone (Sigma-Aldrich, cat. no. P0130) and 25 μM N-acetyl L-Cysteine (Sigma-Aldrich, cat. no. A7250).

E7.5 embryo dissection and ex utero culture—Mouse embryos were obtained from non-hormone primed pregnant mice sacrificed by cervical dislocation at E7.5. Subsequently, embryos were dissected out from the uterus in dissection medium pre-equilibrated at 37° C. for 1 hour, consisting of DMEM (GIBCO 11880; includes already 1 mg/mL D-glucose and pyruvate, without phenol red and without L-glutamine) supplemented with 10% Fetal Bovine Serum (Biological Industries; 040131A), sterilized by using a 0.22 μm filter (JetBiofil; FCA-206-250). The embryos were carefully dissected from the decidua and parietal yolk sac leaving the intact ectoplacental cone attached to the egg cylinder. Briefly, the decidua was isolated from the uterine tissue and the tip of the pear-shaped decidua was cut. The decidua was then opened into halves by introducing the forceps adjacent to the embryo in parallel to its long axis and subsequently opening the forceps. Afterwards the embryo was grasped from the decidua and the parietal yolk sac was peeled off the embryo using two forceps. Embryo dissection was performed on a microscope equipped with a Tokai Hit thermo plate at 37° C., within a maximum of 30 minutes to avoid affecting the embryo developmental potential. Embryos in the neural plate/early head fold stage that showed no evidence of damage in the epiblast were selected for culture. Developmental stage of the embryos was determined according to Downs & Davies⁵. Ex utero embryo culture media (EUCM) was pre-heated for at least an hour by placing it inside a glass bottle on the rotating culture. Immediately after dissection, groups of 5-6 embryos were transferred into glass culture bottles (B.T.C. Engineering—Cullum Starr Precision Engineering Ltd—UK) containing 2 mL of EUCM. The bottles were placed on a rotating bottle culture system, rotating at 30 revolutions per minute at 37° C., and continuously gassed with an atmosphere of 5% O₂, 5% CO₂ at 6.5 pounds per square inch (psi). Following 24 hours, groups of 3 embryos were moved to a new bottle containing 2 mL of freshly prepared media supplemented with extra 3 mg/mL of D-glucose (J.T. Baker) (in addition to the 1 mg/ml glucose found in the base DMEM media), and a gas mixture of 13% O₂, 5% CO₂. At 48 hours of culture, embryos were transferred to a new bottle (2 embryos per bottle) with fresh media supplemented with 3.5 mg/mL of glucose and cultured in a gas atmosphere of 18% O₂ and 5% CO₂. Following 72 hours of culture, each embryo was moved to an individual bottle with 1.5 mL of fresh media plus 4 mg/mL of glucose, with a gas supply of 21% O₂ and 5% CO2. For media exchange, culture media was pre-heated for at least an hour by placing it inside a glass bottle on the rotating culture with an adequate gas atmosphere depending on the stage of the cultured embryos. Embryos were imaged each day using a Discovery V.20 stereoscope (Carl Zeiss). To optimize culturing conditions, different media, glucose concentrations, oxygen concentrations and gas pressures were tested. For paternal imprinting experiments, littermate embryos lacking the reporter allele were used as negative control. For teratogenic experiments, 1 mM valproic acid (Sigma-Aldrich, P4543) diluted in water was added directly to the culture media during media pre-heating.

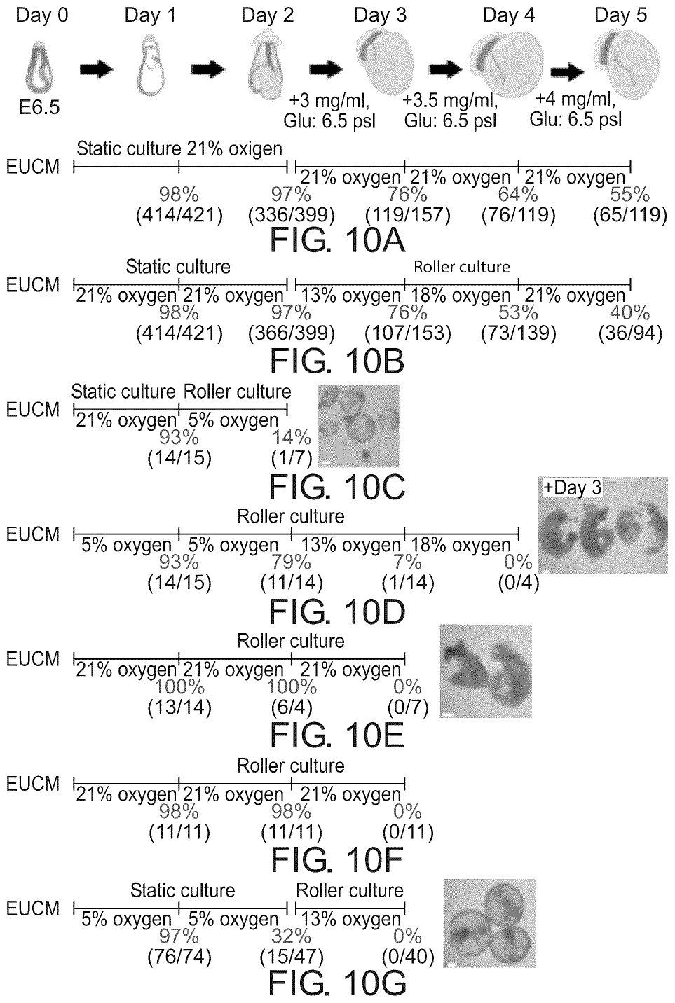

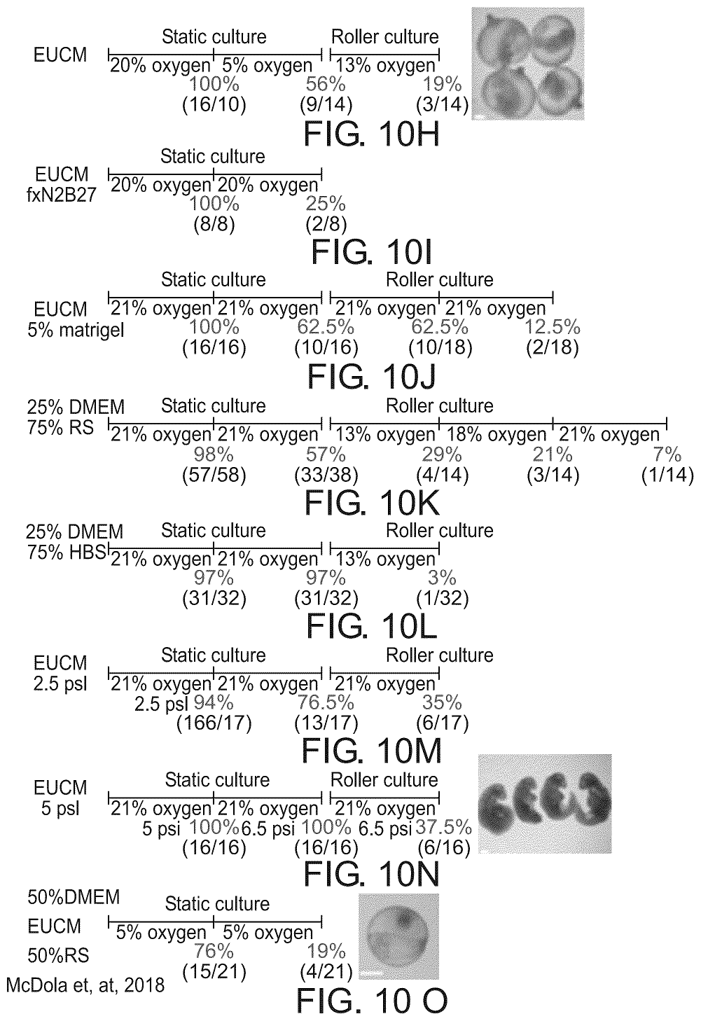

E5.5 and E6.5 embryos ex utero culture—Cultures starting with pre-gastrulation (E5.5) and early gastrulation (E6.5) embryos were effected in static culture conditions until the early somite stage. Embryos were dissected out of the uterus and individual embryos were transferred into each well of a 8-wells glass bottom/ibiTreat μ-plates (iBidi; 80827/80826) filled with 250 μl of EUCM. To optimize culturing conditions, different media, oxygen concentrations (5% or 21%), extracellular matrices (Matrigel), supplements (N2/B27), and gas pressures (hyperbaric 2.5 or 5 psi) were tested. Media was pre-heated for an hour in an incubator with 5% CO2 at 37° C. Pre-primitive streak stage embryos (distal and anterior visceral endoderm stage) were chosen for culture in the case of E5.5, and early-primitive streak stage embryos were selected for cultures beginning at E6.5. Only embryos with no evident damage and without Reichert's membrane were cultured. Half a volume of media was replaced every 24 hours. Embryos were transferred into the rotating culture at the 4-7 somite stage (three days for cultures started at E5.5 and two days for cultures started at E6.5) or at the late gastrulation stage using the same conditions described previously for E8.5, with the difference that embryos were maintained in a constant atmosphere of 21% oxygen and 5% CO₂. Transfer of the embryos at earlier or later stages results in failure of further development. Dynamic oxygen conditions yielded slightly less efficiency for expanding E6.5-E11 than using constant 21% oxygen (FIGS. 10A-B). This difference could result from oxygen diffusion in static conditions being be less efficient than in roller conditions, and that might be why higher oxygen is needed to be delivered in protocols that include static conditions. To allow further culture of the embryos until the stage (E13.5), oxygen was increased to 95% from E10.5 onwards and at E11.5 the embryos were dissected out of the yolk sac and amnion carefully avoiding rupture of any major yolk sac blood vessels, but keeping the yolk sac and umbilical cord attached to the embryo.

Whole-mount immunostaining of E5.5-E8.5 mouse embryos—Embryos grown ex utero and in utero were dissected, removing the Reichert's membrane for E6.5-E7.5 embryos, or the yolk sac and amnion for E8.5 embryos, washed once with 1×PBS, then transferred to ibidi glass bottom 8-well slides (iBidi) and fixed with 4% PFA EM grade (Electron microscopy sciences, 15710) in PBS at 4° C. over-night. Embryos were then washed in PBS for 5 minutes 3 times, permeabilized in PBS with 0.5% Triton X-100/0.1 M glycine for 30 minutes, blocked with 10% normal donkey serum/0.1% Triton X-100 in PBS for 1 hour at room temperature (RT), and incubated over-night at 4° C. with primary antibodies, diluted in blocking solution. Following, embryos were rinsed 3 times for 5 minutes each in PBS/0.2% Triton X-100, incubated for 2 hours at room temperature with secondary antibodies diluted 1:200 in blocking solution (all secondary antibodies were from Jackson ImmunoResearch), counterstained with DAPI (1 μg/ml in PBS) for 10 minutes, and washed with PBS for 5 minutes 3 times. If necessary, yolk sacs separated from the embryos were fixed and stained following this protocol.

iDISCO immunostaining of E9.5-E13.5 mouse embryos—Clearing of embryos from E9.5 to E11.5 was performed according to Renier et al.³¹ with some modifications. Following blocking, embryos were incubated with primary antibodies diluted in PBS/0.2% Tween-20 with 10 μg/ml heparin (PTwH)/5% DMSO/3% Donkey Serum at 37° C. [E9.5/E10.5=24 hours; E11.5=48 hours (72 hours for Sox17 and Foxa2 antibodies)]. Afterwards, samples were washed in PTwH for 24 hours (15 minutes, 30 minutes, 1 hour, 2 hours, and overnight washes), and incubated with adequate secondary antibodies (1:200) diluted in PTwH/3% Donkey Serum at 37° C. for 48 hours. For human cell specific NUMA staining, donkey anti-rabbit Biotin and Streptavidin-Cy3 (each incubated overnight) were used for signal enhancement. Following, embryos were incubated for 30 minutes with DAPI (1 μg/ml) diluted in PTwH, washed in PTwH for one day (5 minutes, 15 minutes, 30 minutes, 1 hour, 2 hours, and overnight washes) and dehydrated in methanol/H₂O series (1 hour each), and then incubated overnight in 100% methanol. Embryos were incubated in 66.6% DCM/33.3% methanol on shaker for 3 hours, followed by 100% DCM (Sigma; 270997) for 5 minutes, and finally cleared and stored in Benzyl Ether (Sigma; 108014).

Statistical analysis—All statistical analysis were performed using the GraphPad Prism 8 software (La Joya, California). In all cases, data on graphs indicates means plus s.e.m. of a minimum of two independent experiments. Kolmogorov-Smirnov test was performed to check normal distribution of data before each statistical test. Significant difference between two samples was evaluated by unpaired two-sided Student's t-test if data was normally distributed or Mann-Whitney test for non-normally distributed data. p<0.05 was considered as statistically significant.

Single cell RNA-seq—Ex utero cultured embryos dissected from the maternal uterus at E6.5 were sequenced at two time points (after two days and four days of culture). In utero and ex utero developmentally matched embryos were dissociated using Trypsin-EDTA solution A 0.25% (Biological Industries; 030501B) during 10 minutes and 15 minutes at 37° C., respectively. E8.5 embryos were processed including the yolk sac but removing the ectoplacental cone, while for E10.5 only the embryo proper was processed removing the extraembryonic membranes. Trypsin was neutralized with media including 10% FBS and cells were washed and resuspended in 1×PBS (calcium and magnesium free) with 400 μg/ml BSA. Cell suspension was filtered with a 100 μm cell strainer to remove cell clumps. A percentage of cell viability higher than 90% was determined by trypan blue staining. Cells were diluted at a final concentration of 1000 cells/μL. Each group of embryos at E8.5 (4 ex utero and 4 in utero) was run into two independent channels of the Chromium 10× Genomics chip, the first channel containing an independent embryo while the second channel consisted of three embryos pooled together. All E10.5 embryos (7 ex utero and 5 in utero) were run as independent samples. scRNA-seq libraries were generated using the 10× Genomics Chromium v3 system (5000 cell target cell recovery) and sequenced on Illumina NovaSeq 6000 platform according to the manufacturer's instructions.

Single cell RNA-seq data processing—10× Genomics data analysis was performed with the Cell Ranger 3.1.0 software (10× Genomics) for pre-processing of raw sequencing data, and Seurat 3.0^{32,33} for downstream analysis. The mm10-3.0.0 gene set downloaded from 10× was used for gene reference requirements. To filter out low expressing single cells, possible doublets produced during the 10× sample processing, or single cells with extensive mitochondrial expression, we filtered out cells with under 200 expressing genes, over 4000 expressing genes and over 15% or 10% mitochondrial gene expression in E8.5 (day 2) and E10.5 (day 4) accordingly. Filtering from E8.5 and E10.5 (accumulated samples of in utero and ex utero), reduced cell count from 16317 to 10707 cells and from 64543 to 63481 cells, respectively. Seurat integrated analysis and anchoring of all individual samples was performed and then normalized by log-normalization using scale-factor-10000. Top 2000 variable genes were established by variance stabilizing transformation method, and subsequently scaled and centered. PCA analysis was performed for dimensional examination using “elbow” method. The first 15 dimensions showed the majority of data variability. Therefore, UMAP dimensional reduction was performed on the first 15 dimensions in all samples. The parameters for fold-change-threshold used for identification of differentially expressed genes were log(0.25) and min.pct=0.25 for both embryos at E8.5 and E10.5. For cluster annotation, the area under the curve (AUC) methodology was conducted to identify the enrichment of each annotated gene-set to each individual single cell. The annotations were based on gene annotations published in¹⁹ for E8.5 (day 2) embryos and on the Mouse Organogenesis Cell Atlas²¹ for E10.5 (day 4) embryos, and performed using the R package AUCELL 1.10.0³⁴, using parameters: aucMaxRank=100 (5% of the total gene count) under the AUCell_calcAUC function. Each cell was then annotated to a single tissue based on its highest AUC score prediction. Each tissue was then cross tabulated with each cluster to assess cluster-tissue overlap, and additionally normalized by z-score, and ranged to 0-1 for plotting purposes. Next, to evaluate the probability of a certain cluster to be enriched to a certain tissue, the annotated AUC predictions of each cell to a tissue were utilized to compare to the observed cluster annotation of each cell, thus producing a p-value based on Mann-Whitney U statistics. This was performed using the R package roc.area v1.42 (CRAN.R-project.org). Integration of both the predicted annotation overlap and its statistical enrichment to each cluster, resulted in a single predicted tissue per cluster. Differential expression (DEGs) of compatible clusters between in utero and ex utero was performed using parameters: fold-change-threshold of log(0.5) and with min.pct=0.25. DEGs with significant values were also enriched using the gene ontology database via R package limma 3.42.2³⁵ using function “goana”. To assess significant changes in the proportional size of each cluster between in utero and ex utero in E10.5, t.test of the proportional size of each cluster was evaluated and corrected using Bonferroni correction, comparing the two groups.

Morphological evaluation of mouse embryo development—Assessment of appropriate embryo development was performed based on previously established morphological features [Van Maele-Fabry, G., et al. Toxicol. Vitr. 4, 149-156 (1990); Van Maele-Fabry, G., et al. Int. J. Dev. Biol. 36, 161-167 (1992)]. Only embryos presenting all of the following features were considered as properly developed.

Proper development at the morphological level from E7.5 to E11 was assessed as follows: Culture day 1 (E8.5): >4 somites, embryo curved dorsally, amnion and yolk sac are enclosing the embryo, the allantois extended into the exocoelom and started to fuse with the chorion, the circulatory system differentiated and blood circulated through the vessels encircling the yolk sac and in the embryo, beating horseshoe-like heart rudiment and foregut pocket visible in the frontal part of the embryo, closing but unfused neural folds. Culture day 2 (E9.5): >20 somites, forelimb buds clearly present, axial turning of the embryo leading the dorsal part to face outside (C-shaped embryo); establishment of the umbilical cord connected to the placental cone, plexus of yolk sac blood vessels observed, three-chambered heart, posterior neuropore closing with small opening remaining, cranial part of the neural tube closed, brain regionalized into forebrain, midbrain and hindbrain, otic pit present and separated from the epidermis, the maxillary process, mandibular and hyoid branchial arches are visible, development of the optic vesicle. Culture day 3 (E10.5): >33 somites, tail bud and hindlimb buds clearly present, paddle-shaped forelimbs, posterior neuropore closed, visible division between telencephalon, diencephalon, mesencephalon, metencephalon and myelencephalon to form a five-vesicles brain, four-chambered heart, invaginating optic vesicle, olfactory plate formed, vessels of the yolk sac form a hierarchical network of large and small-caliber vessels with red blood cells circulating around the yolk sac and the body of the embryo, formation of the fourth branchial arch. Culture day 4 (E11): at this stage the embryos display all the features assessed at E10.5 plus developed nasal pits, invagination and closure of the lens vesicle, and paddle-shaped hindlimbs. For calculating efficiency of ex utero culture, the total number of embryos assessed per condition in every sampled timepoint is indicated. Embryos dissected, fixed or moved to other conditions at any point during the time-course, are subtracted from the total where relevant.

Proper development at the morphological level from E5.5/E6.5 to E8.5 was assessed as follows: At E6.5, the embryos are constituted by three cell lineages: the cup-shaped pluripotent epiblast (Epi) and two extra-embryonic lineages, the extraembryonic ectoderm (ExE) and the visceral endoderm (VE). The cavities in the embryonic and extraembryonic compartments are unified to form the pro-amniotic cavity, radial symmetry is broken in the epiblast to initiate specification of the primitive streak. After 1 or 2 days of culture ex utero (depending on the time of culture initiation, E5.5 or E6.5), the embryos reach the neural plate stage equivalent to E7.5, with no allantoic bud or early bud. The amniotic folds fuse to form the amnion, and the chorion develops from the ExE and the extraembryonic mesoderm. These events generate three cavities in the embryo: amniotic, exocoelomic and ectoplacental cavities. A small allantois bud is present in some of the embryos, at the base of the primitive streak, and the anterior ectoderm begins to form the future neural groove. On the following day, cultured embryos present between 4 to 8 pairs of somites with the embryo curved dorsally, the yolk sac and yolk sac blood circulation has been established, the allantois started to fuse with the chorion, head folds are well-formed as well as the invaginating foregut and beating heart.

Proper development at the morphological level to E12.5 and E13.5 was assessed as follows: At E12.5, the retina develops pigmentation and both forelimbs and hindlimbs acquire a paddle-shape. At E13.5 exhibit the earliest sign of digits and 50-55 somites formed, 5 rows of whiskers and umbilical hernia clearly apparent.

Assessment of embryonic length—Morphometric measurements were performed using the CellSens Entry 1.18 software (Olympus) by using the images of the embryos acquired every day. Length of the antero-posterior axis was measured for embryos between E6.5 to E8.5. The crown-rump length (the longest straight line from the cranial to the caudal end of the body) was measured for embryos at later stages (E9.5 to E11) after removing the yolk sac. Length of cultured embryos was compared with freshly dissected in utero embryos at matched embryonic stages.

Culture of mouse naive and primed embryonic stem cell lines—For generation of Epiblast Stem cells (EpiSCs), V6.5 CAGGS-EGFP cells were cultured on mouse embryonic fibroblast (MEF) feeder cells for more than 5 passages under standard EpiSC conditions as previously described³. Before injection, EpiSC lines were cultured on matrigel for 1-3 passages and treated with 10 μM ROCKi (Y-27632) the day before. For naïve conditions, parental CAGGS-EGFP ES cells were passaged in standard N2B27 2i/LIF conditions [Bayerl, J. et al. bioRxiv 2020.05.23.112433 (2020). doi:10.1101/2020.05.23.112433]. For generation of Epiblast-like Stem Cells (EpiLCs), CAGGS-EGFP V6.5 naïve 2i/LIF ES cells were transferred for 48 hours into priming medium+1% KSR on Matrigel. Formative EpiLC state was validated by PGCLC induction competence as previously described [Kinoshita, M. et al. Cell Stem Cell (2020). doi:10.1016/j.stem.2020.11.005]. Cells were prepared for injection by digestion with trypsin/EDTA 0.25% for 5 minutes followed by dilution in FBS-containing DMEM, washed twice in 1×PBS and filtered through a 70 μm cell strainer. Finally, cells were suspended in the respective media. Cell lines were routinely checked for Mycoplasma contaminations every month (Lonza MycoAlert Kit), and all samples analyzed were not contaminated.

Whole E8.5 embryo electroporation—Mouse E7.5 embryos were cultured for 24 hours and subsequently microinjected with 0.1-0.5 μl of PCAGs-EGFP plasmid into the neural tube. For this purpose, individual embryos were transferred from the roller culture to a plate filled with dissection media pre-heated at 37° C., and each embryo was held in parallel along its antero-posterior axis with forceps and injected using a micromanipulator, taking care that the yolk sac remains intact after injection. Plasmid was diluted at 1 μg/μl in PBS and mixed with 1% fast green dye (1 mg/ml) (Sigma; F7258). Immediately after injection the embryo was transferred into PBS and electroporated using a NEPA21 super electroporator equipped with round platinum plate electrode tweezers (NepaGene). A range of voltages were tested (10-30V) to optimize the conditions that allow good plasmid integration efficiency and embryo viability after electroporation. The most optimal conditions were as follows: Two poring pulses applied at 20V with a duration of 30 milliseconds (ms) each, a pulse interval of 450 ms and a decay rate of 10%, followed by five transfer pulses applied at 15V for 50 ms each with an interval of 450 ms between pulses and a voltage decay of 40%. Electroporated embryos were cultured for additional one to three days. Total number of reporter-expressing cells was measured using the cell counter Plugin in Fiji/ImageJ.

Lentiviral transduction of ex utero embryos—For the generation of lentivirus, HEK293T cells were plated in 10 ml DMEM, containing 10% FBS and Pen/Strep in 10 cm dishes, in aliquots of 3 million cells per plate. On the next day, cells were transfected with the third generation Addgene lentivirus vectors (0.8 μg of pRSV-Rev (Addgene 12253), 0.8 μg of pMDLg/pRRE (Addgene 12251), 1.6 μg of pMD2.G (Addgene 12259), using jetPEI™ transfection reagent, along with 16 μg of the target plasmid FUGW (the pRSV-Rev and pMDLg/pRRE are the packaging vectors and pMD2.G is the envelope plasmid). The medium was replaced after 6 hours of transfection. The supernatant containing the virus was collected 48 hours and 72 hours following transfection, filtered using 0.45 m filter and concentrated by ultracentrifugation for 2 hours at 25,000 rpm (RCF avg: 82,705; RCF max: 112,700). The final viral pellet was resuspended with cold PBS. For lentiviral transduction, embryos were dissected at E6.5 and transferred to a new plate filled with dissection media pre-heated at 37° C. The injection needle was mounted on a mouth pipette and filled by aspiration with concentrated lentiviral vector FUGW (titer was estimated to be 2-3×10⁹ TU/ml). Lentiviruses were delivered by microinjection of 0.1 μl fresh lentiviral solution into the amniotic cavity. Subsequently the embryos were transferred to EUCM and cultured for up to 5 days according to the protocol described above.

Generation of post-implantation intraspecies chimeric embryos by microinjection/graft and ex utero culture—Embryos were dissected from pregnant female mice at E7.5 and microinjected in pre-warmed dissection media with either mouse in vitro EpiSCs/EpiLCs, or with cells directly transplanted from the epiblast of developmentally matched embryos. For EpiSCs and EpiLCs, Y27632 10 μM (Axon Medchem) was applied the night before injection. Cell clumps (10-25 cells) were manually detached from the plate with a pipette tip and injected into the posterior part of the epiblast with a mouth pipette by using a flat-tip microinjection needle. For efficient incorporation of the injected cells into the epiblast of the recipient embryo, cells were expelled carefully from the needle as the micropipette was drawn out of the embryo. For transplantation experiments, clumps of 10-25 cells were cut with a tungsten filament from the epiblast of E7.5 embryos that express td-Tomato ubiquitously (ICR females crossed with Gt(ROSA)26Sor^{(CAG-tdTomato)Hze}/Stra8-iCre), and immediately grafted orthotopically by using a flat microinjection needle mounted on a mouth pipette. tdTomato negative embryos from the same litter and ICR×BDF1 matched embryos were used as recipients of the graft. Following, injected embryos were cultured in rotating bottles for up to four days according to the protocol described for E7.5 embryos. Total number of integrated cells was measured using the cell counter Plugin in Fiji/ImageJ.

RNA extraction and qPCR of mouse PSC lines—EpiSCs and EpiLCs lines were characterized by real-time PCR. Briefly, total RNA was isolated using Trizol (Ambion Life Technologies), and 1 μg of total RNA was reverse transcribed using High-Capacity Reverse Transcription Kit (Applied Biosystems). Quantitative PCR analysis was performed with the SYBR™ Green PCR Master Mix (Applied Biosystems) using 10 ng of cDNA per reaction in a Viia7 platform (Applied Biosystems). Fold change was normalized to Gapdh expression. As expected Nanog was decreased upon priming, while Otx2 and Fgf5 primed makers were induced in both types of primed samples. Brachyury was induced in primed, but not formative EpiLC samples, as recently described [Kinoshita, M. et al. Cell Stem Cell (2020). doi:10.1016/j.stem.2020.11.005]. The following primers were used:

Gapdh-Forward:
(SEQ ID NO: 1)
AGTCAAGGCCGAGAATGGGAAG
Gapdh-Reverse:
(SEQ ID NO: 2)
AAGCAGTTGGTGGTGCAGGATG
Oct4-Forward:
(SEQ ID NO: 3)
AGAGGATCACCTTGGGGTACA
Oct4-Reverse:
(SEQ ID NO: 4)
CGAAGCGACAGATGGTGGTC
Nanog-Forward:
(SEQ ID NO: 5)
CTCAAGTCCTGAGGCTGACA
Nanog-Reverse:
(SEQ ID NO: 6)
TGAAACCTGTCCTTGAGTGC
Sox2-Forward:
(SEQ ID NO: 7)
TAGAGCTAGACTCCGGGCGATGA
Sox2-Reverse:
(SEQ ID NO: 8)
TTGCCTTAAACAAGACCACGAAA
Klf4-Forward:
(SEQ ID NO: 9)
GCACACCTGCGAACTCACAC
Klf4-Reverse:
(SEQ ID NO: 10)
CCGTCCCAGTCACAGTGGTAA
Cdx2-Forward:
(SEQ ID NO: 11)
GCGAAACCTGTGCGAGTGGATG
Cdx2-Reverse:
(SEQ ID NO: 12)
CGGTATTTGTCTTTTGTCCTGGTTTTCA
Gata4-Forward:
(SEQ ID NO: 13)
CACAAGATGAACGGCATCAACC
Gata4-Reverse:
(SEQ ID NO: 14)
CAGCGTGGTGGTAGTCTG
Gata6-Forward:
(SEQ ID NO: 15)
CTTGCGGGCTCTATATGAAACTCCAT
Gata6-Reverse:
(SEQ ID NO: 16)
TAGAAGAAGAGGAAGTAGGAGTCATAGGGACA
Brachyury(T)-Forward:
(SEQ ID NO: 17)
CTGTGACTGCCTACCAGAATGAGGAG
Brachyury(T)-Reverse:
(SEQ ID NO: 18)
GGTCGTTTCTTTCTTTGGCATCAAG
Otx2-Forward:
(SEQ ID NO: 19)
CTTCGGGTATGGACTTGCTG
Otx2-Reverse:
(SEQ ID NO: 20)
CCTCATGAAGATGTCTGGGTAC
Fgf5-Forward:
(SEQ ID NO: 21)
CAAAGTCAATGGCTCCCACGAAG
Fgf5-Reverse:
(SEQ ID NO: 22)
CTACAATCCCCTGAGACACAGCAAATA.

Antibody dilutions for whole-mount immunostaining (E5.5-E8.5)—Rabbit monoclonal anti-Brachyury (D2Z3J) (Cell Signaling, 81694) 1:100; Rabbit polyclonal anti-Cdx2 (Cell Signaling, 3977) 1:100; Mouse monoclonal anti-Cdx2 (Biogenex, MU392A-UC) 1:100; Goat polyclonal anti-Gata4 (Santa Cruz, SC-1237) 1:100; Rabbit polyclonal anti-Gata4 (Abeam, Ab84593) 1:100; Rabbit monoclonal anti-Foxa2 (EPR4466) (Abeam, Ab108422) 1:100; Mouse monoclonal anti-Myosin Heavy Chain II (MF-20) (R&D, MAB4470) 1:100; Goat polyclonal anti-Otx2 (R&D, AF1979) 1:200; Rabbit polyclonal anti-Pax6 (Covance, PBR-278P) 1:100; Goat polyclonal anti-Sox2 (R&D, AF2018) 1:200; Rabbit polyclonal anti-Sox9 (Millipore, AB5535) 1:100; Goat polyclonal anti-Sox17 (R&D, AF1924) 1:100; Mouse monoclonal anti-Tubulin B3 (Tuj1) (Covance, MMS-435P) 1:200; Chicken polyclonal anti-GFP (Abeam, Ab13970) 1:250; Mouse monoclonal anti-Oct4 (C-10) (Santa Cruz, SC-5279) 1:100; Goat polyclonal anti-Lefty 1 (R&D, AF746) 1:100, Goat polyclonal anti-mCherry/Tomato (SiCGEN, AB0040-200) 1:200.

Antibody dilutions for iDISCO (E9.5-E11.5)—Rabbit monoclonal anti-Brachyury (D2Z3J) (Cell Signaling, 81694) 1:100; Rabbit polyclonal anti-Cdx2 (Cell Signaling, 3977) 1:100; Mouse monoclonal anti-Cdx2 (Biogenex, MU392A-UC) 1:100; Goat polyclonal anti-Gata4 (Santa Cruz, SC-1237) 1:100; Rabbit polyclonal anti-Gata4 (Abeam, Ab84593) 1:100; Rabbit polyclonal anti-Foxa2 (Abeam, Ab40874) 1:50; Mouse monoclonal anti-Myosin Heavy Chain II (MF-20) (R&D, MAB4470) 1:100; Goat polyclonal anti-Otx2 (R&D, AF1979) 1:200; Rabbit polyclonal anti-Pax6 (Covance, PBR-278P) 1:100; Goat polyclonal anti-Sox2 (R&D, AF2018) 1:200; Rabbit polyclonal anti-Sox9 (Millipore, AB5535) 1:100; Goat polyclonal anti-Sox17 (R&D, AF1924) 1:50; Mouse monoclonal anti-Tubulin B3 (Tuj1) Tuj1 (Covance, MMS-435P) 1:200; Chicken polyclonal anti-GFP (Abeam, Ab13970) 1:250, Goat polyclonal anti-mCherry/Tomato (SiCGEN, AB0040-200) 1:200; Rabbit polyclonal anti-human TEMEM119 (Invitrogen, PA562505) 1:100; Rabbit anti-hNUMA (Abeam, ab84680) 1:100.

Immunohistochemistry—For OCT-staining, embryos were fixed overnight in 4% PFA at 4° C., washed three times in PBS for 10 minutes each and submerged first in 15% Sucrose/PBS and then 30% Sucrose over night at 4° C. The day after, samples were subjected to increasing gradient of OCT concentration in Sucrose/PBS followed by embedding in OCT on dry ice and stored at −80° C. until further processing. Cryoblocks were cut with LEICA CM1950 and washed once with 1×PBS and incubated with 0.3% H₂O₂ for 20 minutes. Following permeabilization with 0.1% Triton X-100 in PBS for 10 minutes, slides were washed three times with 1×PBS for 2 minutes each and blocked in 10% normal donkey serum in PBS in humidified chamber for 20 minutes at room temperature. Slides were then incubated with proper primary antibody diluted in antibody solution (1% BSA in 0.1% Triton X-100) at 4° C. overnight. Sections were then washed three times (5 min each) in 0.1% Triton X-100 in PBS, incubated with appropriate secondary antibodies diluted in antibody solution at room temperature for 1 hour in the dark, counterstained with DAPI for 20 minutes and mounted with Shandon Immuno-Mount (Thermo Scientific, 9990412). The primary antibodies used were the following: Goat polyclonal anti-Gata4 (Santa Cruz, SC-1237) 1:200; Rabbit monoclonal anti-Foxa2 (Abcam, Ab108422) 1:200.

Confocal microscopy—Whole-mount immunofluorescence and iDISCO images were acquired with a Zeiss LSM 700 inverted confocal microscope (Carl Zeiss) equipped with 405 nm, 488 nm, 555 nm and 635 nm solid state lasers, using a Plan-Apochromat 20× air objective (numerical aperture 0.8) for E5.5/E6.5 embryos, and an EC Plan Neofluar 10× air objective (numerical aperture 0.3) for E7.5 to E11.5 embryos. Images were acquired at 1024×1024 resolution. All images were acquired within the following range of parameters: Laser power: 405 nm: 10-20%; 488 nm: 5-20%; 555 nm 10-40%; 635 nm: 30-80%. Gain ranged from 350 to 600. Pixel size was 1.25 μm with a z-step of 15 μm when using the 10× objective, or 0.5 μm with z-step of 5 μm when using the 20× objective. For confocal imaging, iDISCO cleared embryos were mounted in 35 mm glass bottom dishes (In Vitro Scientific, D35201.5N), employing ethyl cinnamate (Sigma, 112372) as imaging solution. For chimeric embryos, all parameters during image acquisition were compared to stained non-injected control embryos, imaged with equal parameters as the injected embryos. Images and maximum intensity projections were processed using Zen 2 blue edition software 2011 (Carl Zeiss) and Adobe Photoshop CS4.

Light-sheet microscopy—3D images of cleared embryos were acquired on a light-sheet microscope (Ultramicroscope II, LaVision Biotec) operated by the ImspectorPro software (LaVision BioTec), equipped with an Andor Neo sCMOS camera (2,560×2,160, pixel size 6.5 μm×6.5 μm) 16 bit, and an infinity corrected setup 4× objective lens: LVBT 4× UM2-BG (LVMI-Fluor 4×/0.3 Mag. 4×; NA: 0.3; WD: 5.6-6.0 mm), with an adjustable refractive index collar set to the refractive index of DBE (1.56). The light sheet was generated by scanning a supercontinuum white light laser (emission 460 nm-800 nm, 1 mW/nm-3 (NKT photonics). The following excitation band pass filters were used: 470/40 nm for Alexa Fluor 488, 560/40 nm for Rhodamine Red-X, and 617/83 nm for Alexa Fluor 647. The light sheet was used at 80% width and maximum NA (0.154). Laser power ranged between 40 to 80%. The emission filters used were: 525/50 for Alexa Fluor-488, 630\75 for Rhodamine Red-X and 690/50 for Alexa Fluor-647. Stacks were acquired using 5 m step-size and a 200 ms exposure time per step. Imaris (Bitplane) was used to create 3D reconstructions and animations of the imaged embryos.

In toto confocal live imaging—Live E6.5 embryos were dissected as described hereinabove and selected for tdTomato expression. Following, embryos were mounted into a droplet of EUCM adhering the ectoplacental cone to the edge of a paper filter (Millipore, AABG01300) attached to a coverslip with vacuum grease. For imaging of neural tube closure, E7.5 embryos were cultured until E9.0, Td-Tomato⁺ embryos at the proper stage were chosen and moved on a droplet of culture media on a paper filter attached to a coverslip. Then, the dorsal anterior part of the embryo was exposed by opening the yolk sac, and the embryos were anchored onto the paper filter by pressing the yolk sac to the filter. After mounting the embryos, a glass-bottomed dish (MatTek, P35G-1.5-20-C) was placed on top of the embryos using vacuum grease drops on the corners of the coverslip for spacing. Subsequently, the dish was carefully inverted, filled with 2 mL of EUCM and placed in a heat- and humidity-controlled imaging chamber (37° C.; 21% O₂, 5% CO₂) of an inverted Zeiss LSM700 confocal microscope. E6.5 to E8.5 imaging was performed using the 405 nm and 555 nm lasers and an EC Plan Neofluar 10× air objective (numerical aperture 0.3) with 0.5× digital zoom out and a resolution of 512×512 pixels. Laser power was 1% for the 405 nm laser and 8% for the 555 nm laser. Gain ranged from 500 to 600. Pixel size was 2.5 μm with a z-step of 25 μm. E9.0 embryos were imaged using the 555 nm laser (8% power) and an EC Plan Neofluar 5× air objective (numerical aperture 0.16) with 0.5× digital zoom out and a resolution of 512×512 pixels. Pixel size was 2.5 μm with a z-step of 50 μm. Time interval was 1 hour for E6.5 to E8.5 embryos, and 15 minutes for E9.0. Movies were processed using Zen 2 blue edition software 2011 (Carl Zeiss) and Fiji/ImageJ.

Mouse blastocyst micromanipulation and culture of chimeric embryos—Mouse naïve V6.5 CAGGS-EGFP ES cells expanded in 2i/LIF conditions were injected into BDF2 diploid blastocysts as previously described [Gafni, O. et al. Nature 504, 282-286 (2013)]. Ten to fifteen injected blastocysts were transferred to each uterine horn of 2.5 d.p.c pseudo-pregnant females. At E7.5 chimeric embryos were dissected out of the maternal uterus and grown in the roller culture for up to three days using the same conditions described for E7.5 embryos.

In vitro derivation of microglial precursors from human ESCs and generation of human-mouse interspecies chimeric embryos—WIS2 human ESCs were cultured in 5% O₂ on mTeSR1 (Stem cell Technologies) on Matrigel-coated plates. Cultures were passaged every 5-7 days by using TrypLE (GIBCO). ROCK inhibitor (Y-27632, 5-10 PM) was added 24 hours prior to and following cell passaging. A CAGGS-EGFP reporter was introduced into the primed human ESC cells by electroporation. Microglia differentiation was performed according to Wilgenburg et.al. [PLoS One 8, e71098 (2013)]. Briefly, a suspension of 10×10⁵ human ES cells per mL was prepared on embryoid body (EB) formation media consisting on mTESR1, 50 ng/ml human VEGF, 50 ng/ml BMP4, 20 ng/ml human SCF and 10 μM Y-27632. Embryoid bodies were generated by centrifugation on U-shaped bottom Nunclon Sphera 96-well plates (ThermoFisher Scientific, 174925). Medium was refreshed every two days. At day four, EBs were transferred to 6-well plates on differentiation medium, consisting on X-VIVO 15 media (Lonza, BE02-060F) supplemented with IL-3 (25 ng/ml), M-CSF (100 ng/ml) penicillin/streptomycin (100 UI/ml), Glutamax 1×, and 50 μM β-mercaptoethanol. Media was exchanged every week and cells in the supernatant started to be harvested after three weeks. Characterization and validation of human ESCs-derived microglial precursors was performed by flow cytometry on a BD FACS-Aria III. Cells were incubated for half an hour with CD34-PE (BioLegend, 561) and CD43-APC (ebioscience, 84-3C1) antibodies (1:50) on PBS/0.5% BSA. CD34/CD43 double positive cells were considered for calculating differentiation efficiency. Once a continuous efficiency above 85% was obtained for at least two weeks, cultured human derived-microglial precursors were used for injections into post-implantation embryos for up to three months. For embryo injection, cells were harvested from the supernatant and treated for 2 minutes with trypLE at 37° C., washed with PBS and resuspended on media consisting of X-VIVO 15 with 10% FBS. Cell were kept on ice until injected. Immediately before injection, a 60 μl drop of cell suspension was placed on a petri dish, cells were harvested by gentle suction under a stereoscope using a mouth pipette and injected into the amniotic cavity of E7.5 mouse embryos on pre-heated dissection media, taking care of introducing the microinjection needle through the exocoelomic cavity to avoid perforating the epiblast, 50-60 cells were injected per embryo. Finally, injected mouse embryos were grown in roller culture settings for 1-4 days according to the protocol described above for E7.5 embryos. Total number of integrated cells was measured using the cell counter Plugin in Fiji/ImageJ. The use of human ESC line follows the approval of Weizmann institute IRB-ESCRO (#1138-1, #856-1).

Bulk RNA-seq library preparation—V6.5 mESCs grown under naïve (ESCs) and primed (EpiSCS) conditions were used for RNA-seq analysis. Total RNA was extracted using the TRIzol-based RNA MiniPrep kit (Zymo Research). mRNA was purified using Poly-A Dynabeads mRNA DIRECT Kit (Invitrogen) and was utilized for RNA-Seq by TruSeq RNA Sample Preparation Kit v2 (Illumina) according to manufacturer's instruction.

Bulk RNA-seq analysis—Bulk RNA-seq was measured from the following samples: mEpiSCs (2 biological replicates) and mESC (2 biological replicates). Reads were trimmed with TrimGalore 0.6.5 (flags --stringency 3--paired) and aligned to GRCm38 genome using STAR aligner (flags --runThreadN 64--genomeLoad). Counts were estimated using HTSeq-count 0.7.2 (flags -q -f bam -r pos -s no -t exon -i gene_name). Normalization and differentially expressed genes were calculated using DESeq2 R package, with default parameters. Differentially expressed genes were selected if their adjusted p-value was smaller than 0.01 and their Fold change was greater than 2. External gene signatures, based on mouse microarray data, were calculated from GSE60603 (PMID 25945737) [Wu, J. et al. Nature 521, 316-321 (2015)]: shortly, processed data was used to calculate t-test. Genes that had t-test p-value <0.05 were included in the gene signature. The overlap between differentially expressed gene signatures was calculated using Fisher exact test.

Mouse zygotes isolation and ex-utero culture—Female mice (5-8-week old ICR) were superovulated by injecting 5 i.u. of pregnant mare serum gonadotropin (PMS), followed by 5 i.u. of human chorionic gonadotrophin (HCG) 46 hours later. These females were then mated with BDF1 studs. Insemination was verified the next morning by the presence of a copulatory plug, this day is considered as the day 0.5. Mouse zygotes were recovered by flushing the oviduct with M2 medium at E0.5. Adherent granulosa cells were removed from zygotes by incubating them in hyaluronidase (300 μg/mL in M2) (Sigma, H3506). The zona pellucida was removed at E4.5 using acidic Tyrode's (Sigma, T1788). Embryos were cultured from E0.5 to E4.5 in Continuous Single Culture Complete (CSCM) with HSA (Fujufilm, 90165 or 90168) or KSOM (Embryomax KSOM-Mouse Embryo Media SigmaAldrich 32160801) and later transferred to the enhanced in vitro implantation protocol from day 4 to 8 at 37° C. in 20% O₂ and 5% CO₂. Briefly, blastocysts were transferred into 8-well ibiTreat plastic p plates (iBidi) and cultured for 2 days in a modified IVC1 media (Bedzhov et al. Cell 2014) [(Advanced DMEM/F12 (GIBCO, #12634-010) containing 20% Fetal Bovine Serum (FBS) (Biological Industries) and supplemented with 1× Glutamax (GIBCO, #35050-038), penicillin (25 units/ml)/streptomycin (25 mg/ml) (Biological industries, 030311B), 1×ITS-X (ThermoFisher, #51500056), 8 nM β-estradiol (Sigma, #E8875), 200 ng/ml progesterone (Sigma, P0130 #), and 25 μM N-acetyl-L-cysteine (Sigma, #A7250)], which was further supplemented with 100 nM 3,3′,5-Triiodo-L-thyronine (T3) (SIGMA, #T6397), referred to herein as “enhanced IVC1 (EIVC1)”. In some experiments the enhanced IVC1 was further supplemented with 0.22% sodium lactate (SIGMA, #L7900) and an extra 1 mM sodium pyruvate (Sigma-Aldrich, cat. no. P4562). At day 6, media was replaced with 250 μl of a medium referred to herein as “enhanced IVC2 (EIVC2)”, which is similar to enhanced IVC1, but contains 30% human umbilical cord blood serum instead of FBS or KSR and optionally further supplemented with 1× N2 supplement (ThermoFisher, #17502048) and 0.5× B27 supplement (ThermoFisher, #17504044). As a reference the IVC2 medium described in Bedzhov et al. was devoid of serum and contained 30% KSR knock-out serum. Embryos were cultured in the enhanced IVC2 medium for 2 days (until day 8), replacing half of the media after one day. Alternatively, when indicated, the blastocysts were cultured for 2 days with EUCM2, followed by 1 day with EUCM3 and 1 additional day with EUCM4. From culture day 8 onwards the media was replaced by 250 μl of Ex Utero Culture Media (EUCM). Half volume of media was refreshed daily. At culture day 9 or 10 the plate was placed on a shaker rotating at 60 rpm for 24 hours. Following, the embryos were transferred into the roller culture as described above and maintained in a constant atmosphere of 21% oxygen and 5% CO₂ until day 13. Of note, culturing the embryos through the implantation blastocyst (E4.5) until the post implantation pre gastrulation stage (E5.5) resulted in a 2 days delay in development in comparison to an in utero counterpart. To overcome this delay, an alternative protocol utilized a Lykos Laser system by Hamilton Thorne in order to excise the mural trophectoderm of the blastocyst by laser microdissection according to Ozguldez and Bedzhov (2021) PMID: 32944901, leading to release of intra-blastocyst fluid and pressure prior to culturing. Bright field pictures of the embryos were taken every 24 hours using a Discovery V.20 stereoscope (Carl Zeiss). Emerging egg cylinders were washed twice in PBS, fixed with PFA 4% and immunostained to evaluate appropriate development.

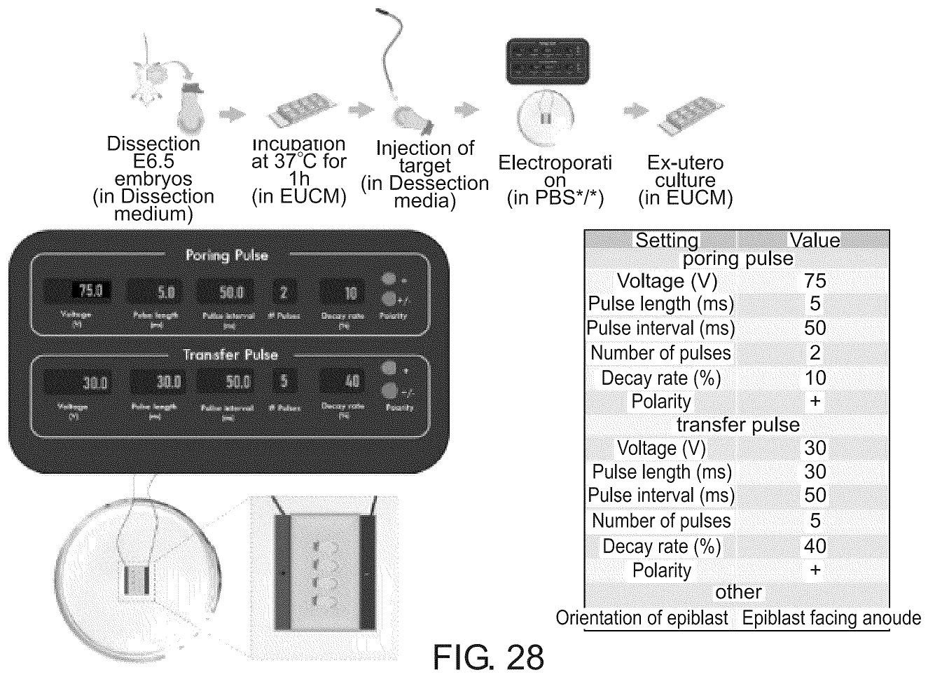

Whole E6.5 embryo electroporation—Mouse E6.5 embryos were cultured for 1 hour in EUCM at 37° C. in iBidi plates to minimize the stress. Following, CRISPR RNAs were injected using a mouth pipet (aspirator tube assembled to a microcapillary) into the pro-amniotic cavity. This was done by transferring the embryos from the iBidi plate to a 60×15 mm Petri dish filled with Dissection Medium. Following, the embryos were transferred to an electroporation chamber (CUY520P5, Nepagene) filled with PBS^{+/+} and connected to a Super Electroporator Nepa21 Type II (Nepagene). Electroporated embryos were cultured according to the above described ex-utero culture protocols. Optimizations for electroporation were conducted with a GFP plasmid: 3 μg/μL (pmaxCloning™ Vector, LONZO, Catalog #VDC-1040) and/or Atto-labelled tracrRNA: 2 ug/uL (Alt-R Cas9 tracrRNA, ATTO 550, IDT, Cat. 1073190). The most optimal conditions are shown in FIG. 28.

For knocking out Lim1, Pax6 or Mesp1, CRISPR sequences were annealed to tracrRNA to generate guide RNA complex by mixing equal volumes of 100 μm crRNA and 100 μm tracrRNA and annealing in a thermocycler (95° C. for 5 minutes and then ramp down to 25° C. at 5° C./minute). The following CRISPR sequences were used:

mLim1_cr1_Frw-
(SEQ ID NO: 23)
caccgggagaagcacttctcggtc;
mLim_cr2-
(SEQ ID NO: 24)
atgtagagctcctcgccggc;
mPax6_cr1_F-
(SEQ ID NO: 25)
caccgtggtgtctttgtcaacggg;
mPax6_cr2-
(SEQ ID NO: 26)
acacttactgttctgcatgc;
mMesp1_cr1_F-
(SEQ ID NO: 27)
caccgagccaccgatgccttccgat;
mMesp1_cr2-
(SEQ ID NO: 28)
gccgctgtccgctacccagg.

Whole E6.5 embryo lentiviral infection—For the generation of lentivirus, HEK293T cells (ATCC—CRL1573) were plated in 10 ml DMEM, containing 10% FBS and Pen/Strep in 10 cm dishes, in aliquots of 3 million cells per plate. On the next day, cells were transfected with the third generation Addgene lentivirus vectors (0.8 μg of pRSV-Rev (Addgene 12253), 0.8 μg of pMDLg/pRRE (Addgene 12251), 1.6 μg of pMD2.G (Addgene 12259), using jetPEI™ transfection reagent, along with 16 μg of the target plasmid FUGW. (The pRSV-Rev and pMDLg/pRRE are the packaging vectors and pMD2.G is the envelope plasmid). The medium was replaced after 6 hr of transfection. The supernatant containing the virus was collected 48 hr and 72 hr following transfection, filtered using 0.45 m filter and concentrated by ultracentrifugation for 2 hr at 25,000 rpm (RCF avg: 82,705; RCF max: 112,700). The final viral pellet was resuspended with cold PBS. For lentiviral transduction, embryos were dissected at E6.5 and transferred to a new plate filled with dissection media preheated at 37° C. The injection needle was mounted on a mouth pipette and filled by aspiration with concentrated lentiviral vector FUGW (titer was estimated to be 2-3×10⁹ TU/ml). Lentiviruses were delivered by microinjection of 0.1 μl fresh lentiviral solution into the amniotic cavity. Subsequently the embryos were transferred to EUCM and cultured for up to 5 days according to the protocol described above.

### Example 1

### Ex-Utero Culturing a Whole Mouse Embryo from E7.5 to Advanced Organogenesis (E11)

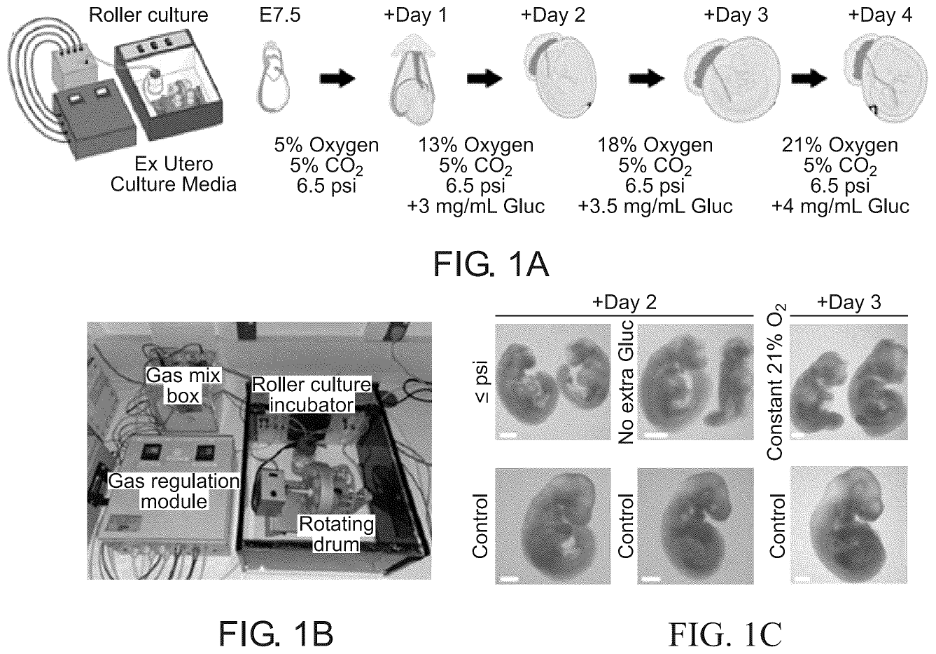

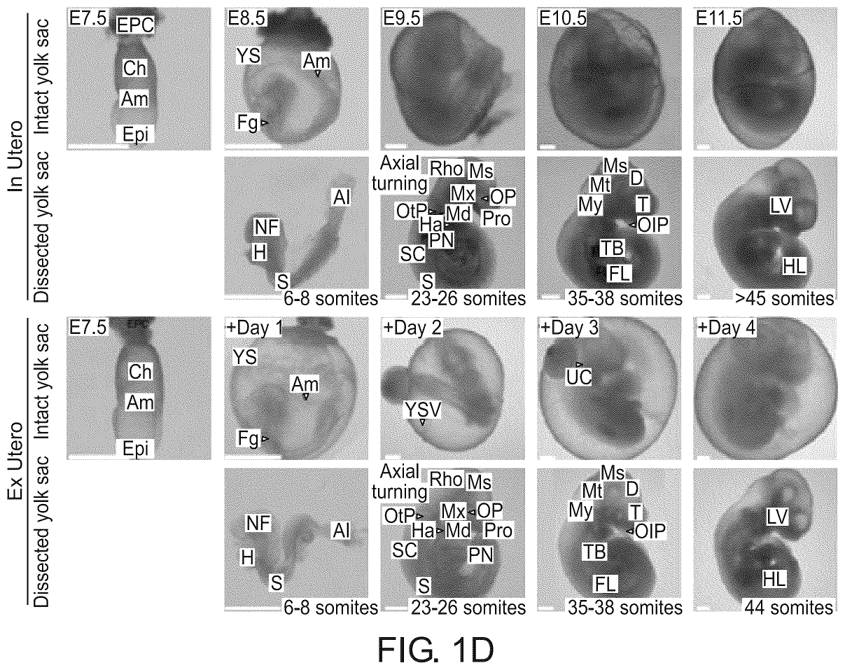

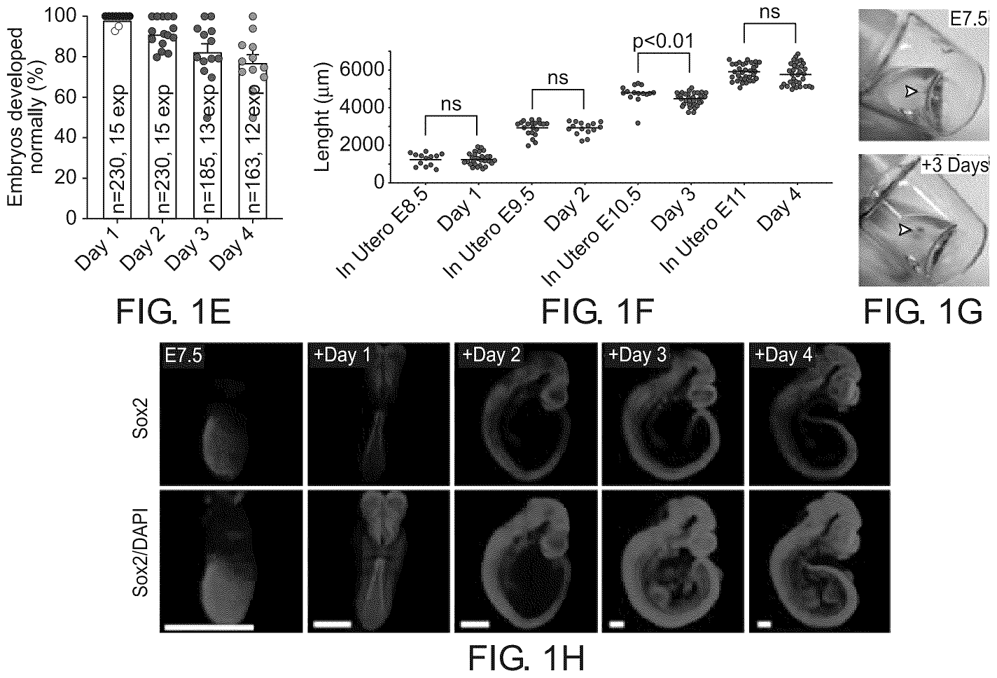

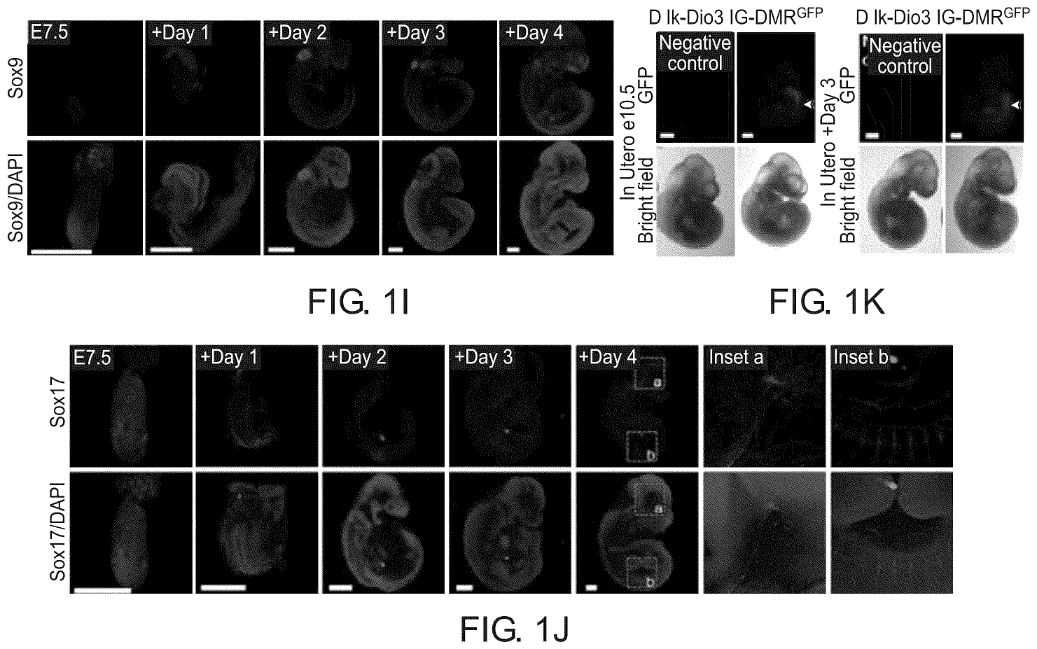

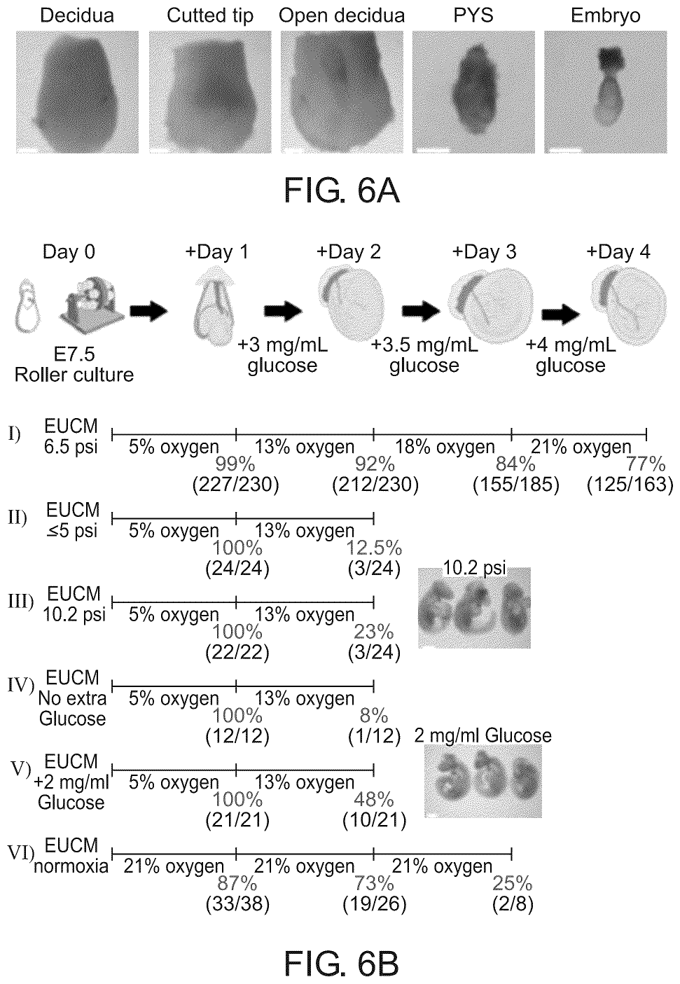

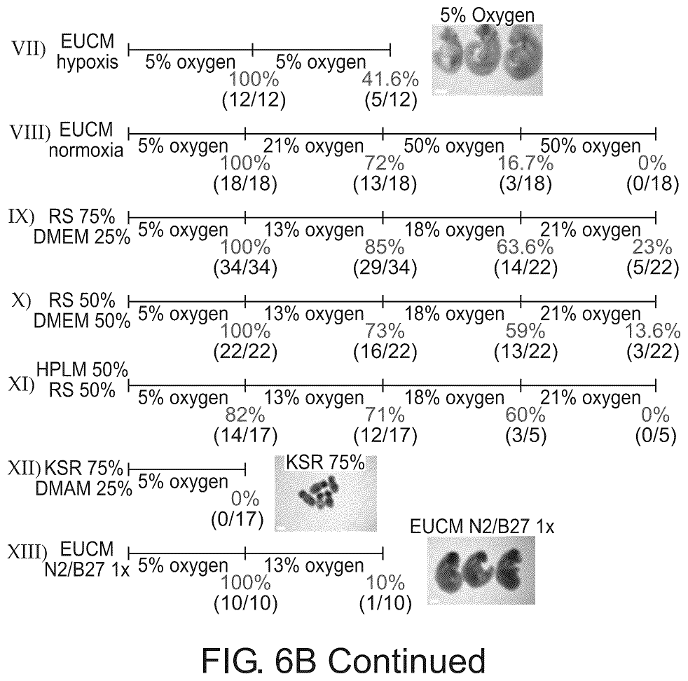

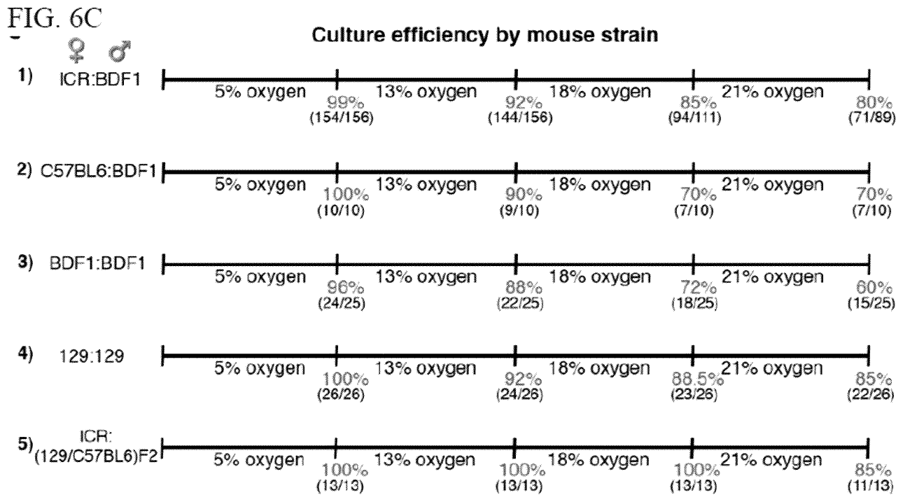

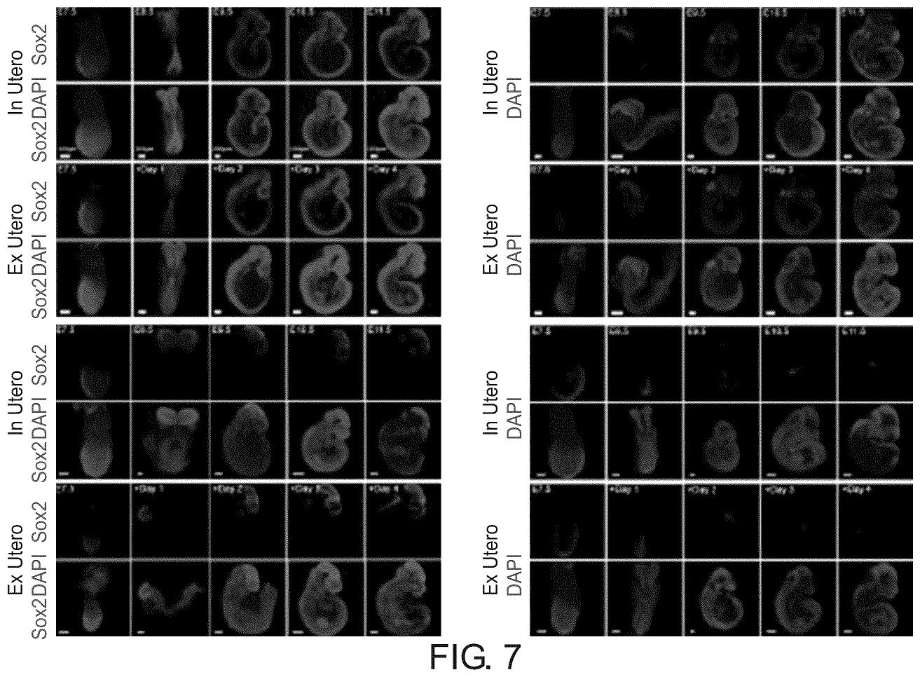

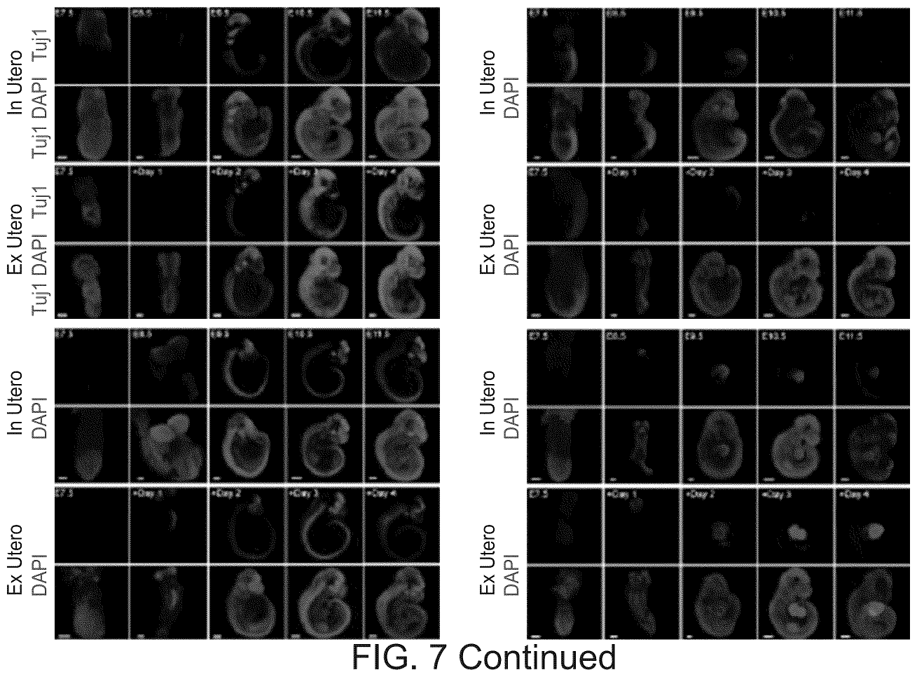

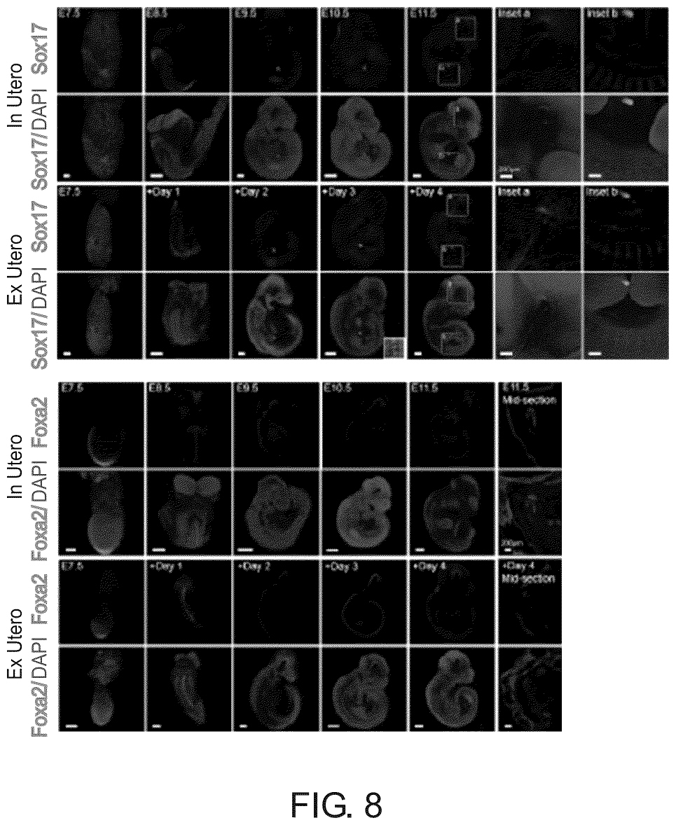

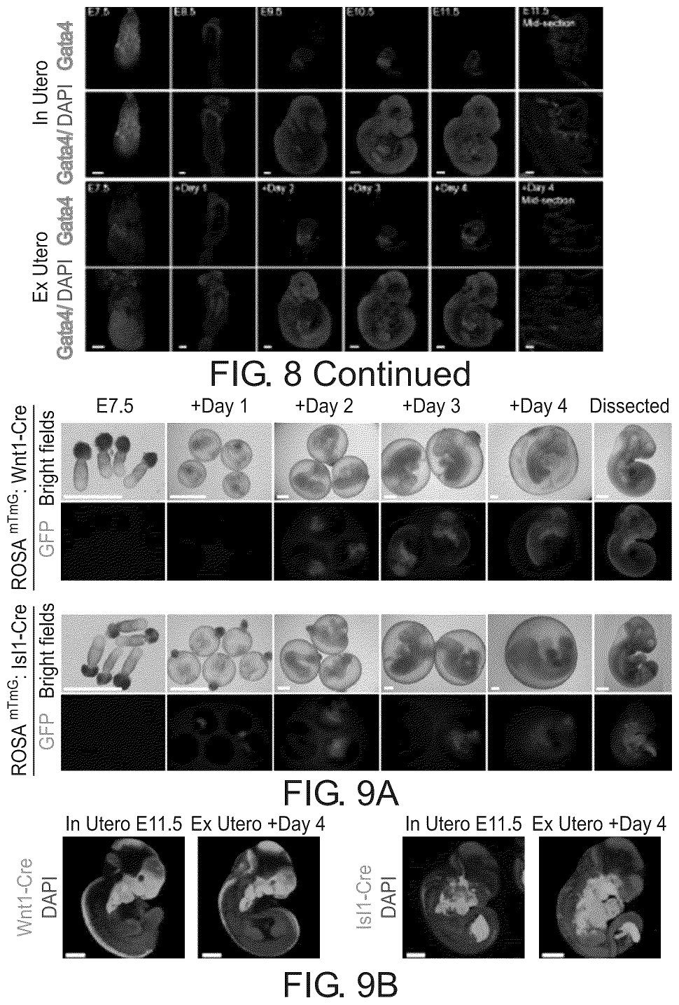

The present inventors set out to test whether some of cell culture supplements or biomechanical principles newly established in stem cell research, could be helpful for establishing stable and efficient protocols for extended culturing of pre-gastrulating mouse embryos all the way until advanced organogenesis stages (e.g. hyperbaric chambers, synthetic sera¹²). To this end the “roller culture system” on a drum was utilized and was integrated with a customized and in house developed electronic gas regulation module that allows precise control not only of O₂ and CO₂ levels with high sensitivity, but also allows controlling the atmospheric pressure (FIGS. 1A-B and 5A-M). The latter was motivated by the ability of pressure to enhance oxygen delivery to tissues and recent studies demonstrating how atmospheric pressure can alter cell growth^{13,14}. Following, conditions that support growth of E7.5 late-gastrulating embryos (neural plate and headfold-stage¹⁵) until the hind limb formation stage (˜E11) with high efficiency were established (FIGS. 1A and 6A-B). First, a media comprising a mixture of 25% DMEM, 50% rat serum (RS) and 25% human umbilical cord blood serum (HCS), designated herein as ex utero culture media (EUCM), consistently supported embryo growth with much higher efficiency than rat serum only (FIG. 6B).

In some embodiments, optionally, the medium further comprises knockout serum replacement (KSR) in addition to the rat serum and the human serum.

In some embodiments, optionally, the KSR partially replaces one of either the human serum, the rat serum or partially replaces a quantity of both.

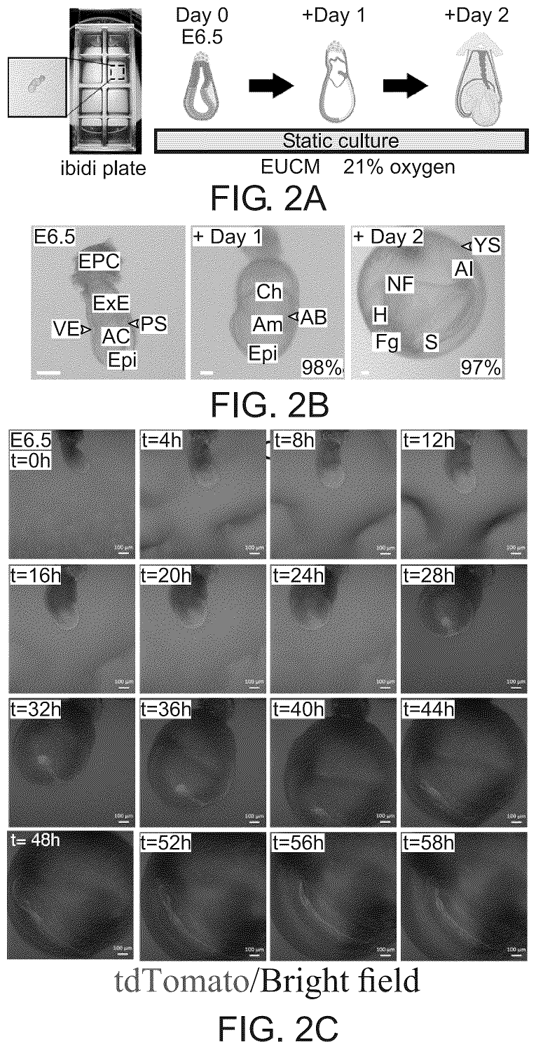

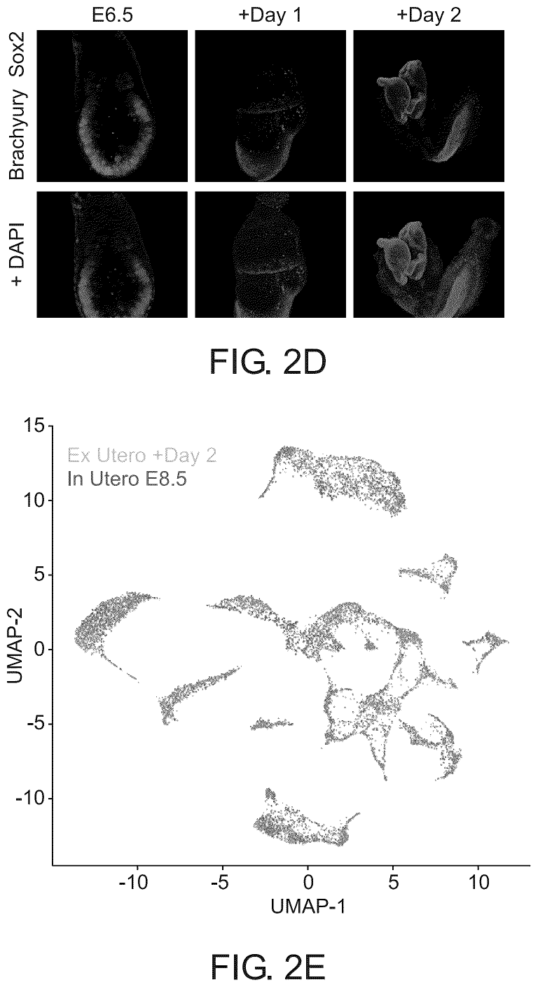

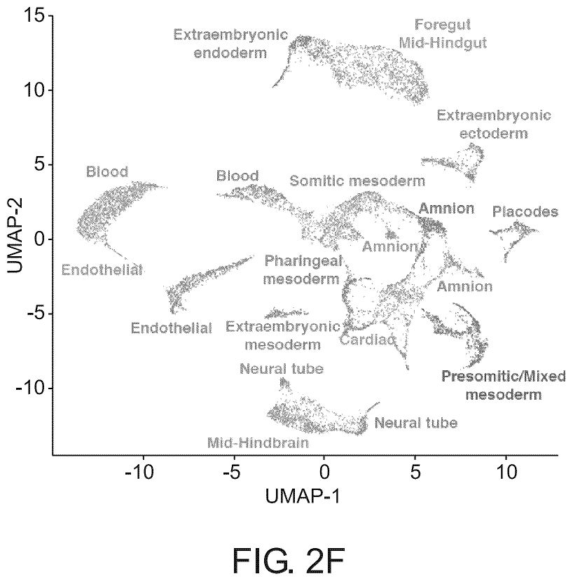

Notably, supplementing EUCM with extra Glucose every 24 hours and until the end of the culture period, was critical for overcoming developmental abnormalities following two days of culture (FIGS. 2C and 6B). Applying sequential increases in the oxygen levels every 24 hours, starting from 5% O₂ at E7.5, 13% at E8.5, 18% at E9.5, and ending with 21% O₂ at E10.5 was most optimal for the robust outcome reported herein (FIGS. 1C and 6B). In addition, maintaining a hyperbaric pressure of 6.5 psi was also critical for normal and efficient development (FIGS. 1C and 6B). This protocol yielded ˜77% normal embryo development following 4 days of culture and in different mouse strains (FIGS. 1D-E and 6C). After 4 days, the embryos started to show abnormalities, yolk sac circulation abruption, pericardial effusion and quickly died overnight. The latter limits are consistent with hydrops fetalis due to insufficient oxygenation and nutrient supply by the ex utero system (given the lack of maternal blood supply in this setting) that no longer matches the increased body size at E11.

To assess appropriate embryo development ex utero, previously defined morphological landmarks¹⁶ were evaluated (FIGS. 1D-F). At the last day of culture, maximum embryo growth was reached at about 44 somites, equivalent to ˜E11 (Theiler stage 18). The length of the cultured embryos was comparable to matched in utero embryos (FIG. 1F). In addition, eleven developmental markers were analyzed, which all showed consistent spatio-temporal gene expression patterns between in utero and ex utero developed embryos (FIGS. 1G-I and 7-8). Mouse transgenic lines expressing the GFP reporter for imprinting erasure of Dlk1-Dio3 intergenic DMR¹⁷ in the migrating primordial germ cells, or under tissue specific promoters (Wnt1-Cre and Isl1-Cre) showed that the GFP expression patterns in the cultured transgenic embryos resemble those of the control in utero samples mouse embryos (FIGS. 1J and 9A-B). These data suggest that the ex utero cultured embryos recapitulate development properly until approximately the 44 somites stage.

### Example 2

Ex-Utero Culturing a Whole Mouse Embryo from E5.5 or E6.5 to E8.5

In the next step, the present inventors aimed to expand the ex utero culture protocol by establishing conditions to grow the mouse embryo from pre-gastrulation stages (E5.5-6.5). Explanted E6.5 embryos grown in rotating bottles either in 5% or 21% O₂, or in previously described static conditions¹⁸ did not develop beyond the early somite-stage (FIGS. 10D, 10E and 10O). Thus, in the search of alternative culture parameters for growing embryos from E6.5 to E8.5 in static conditions (FIGS. 2A and 10A-O). The following conditions were optimized: 25% DMEM/50% RS/25% HCS in 21% O₂. The latter supported the development of early-streak embryos (E6.5) in static culture until the early somite-stage (48 hours) with 97% efficiency (FIGS. 1A-C and 10A-B). In utero and ex utero-grown embryos were equivalent at the morphological level and in the expression of all eleven lineage markers analyzed (FIGS. 2B-D and 11A-C). The robustness of these culture conditions allowed in toto imaging of the gastrulating mouse embryo for up to 58 hours (FIG. 2C).

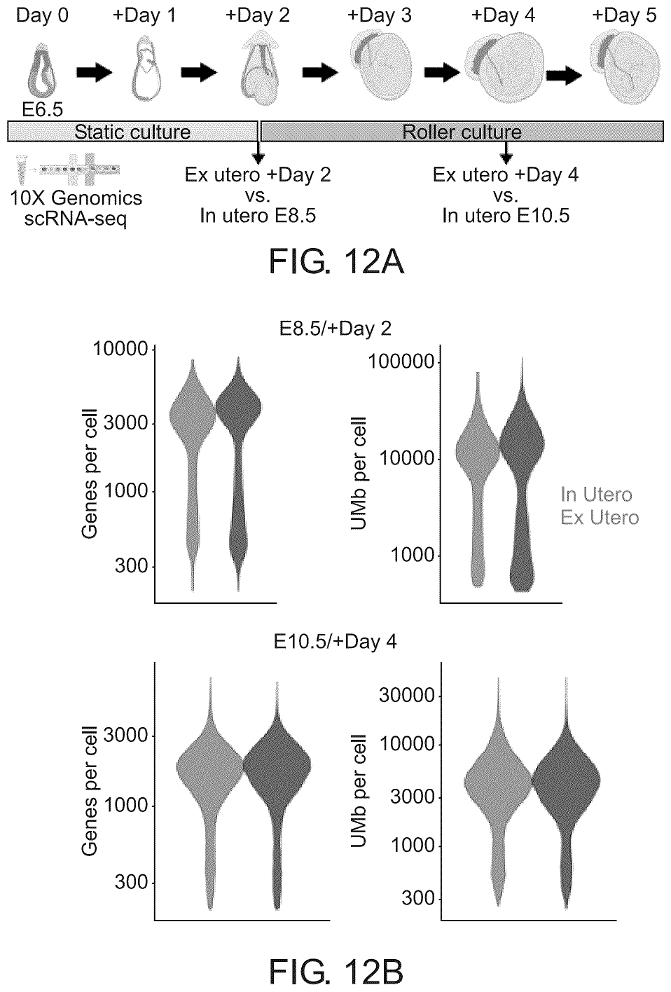

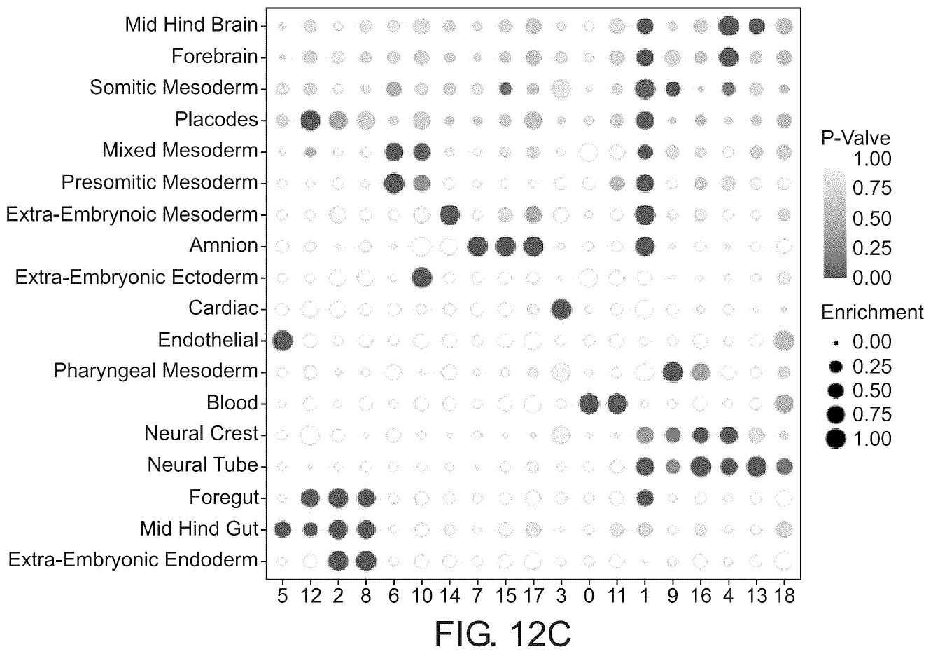

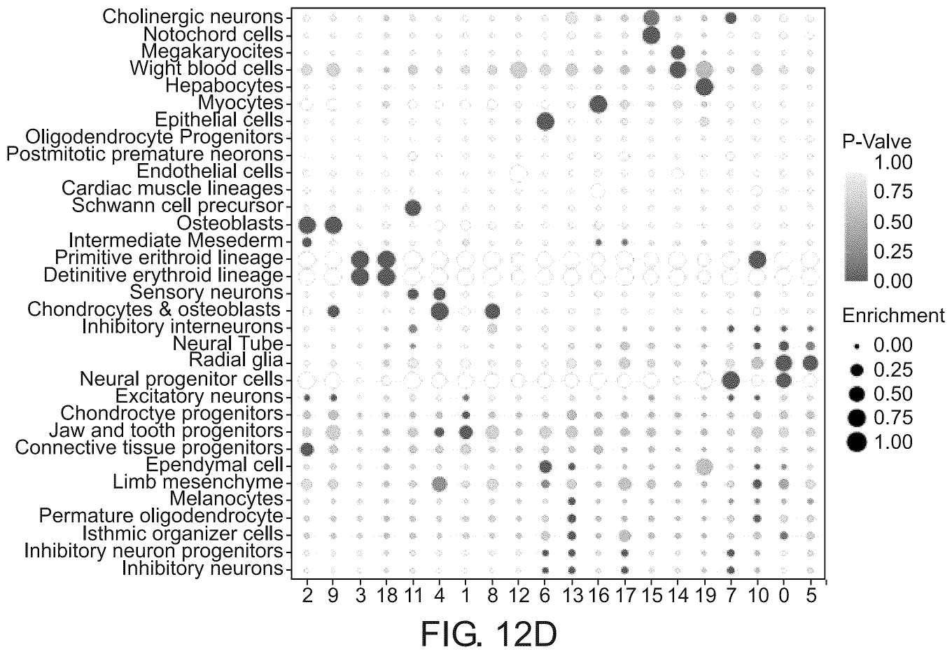

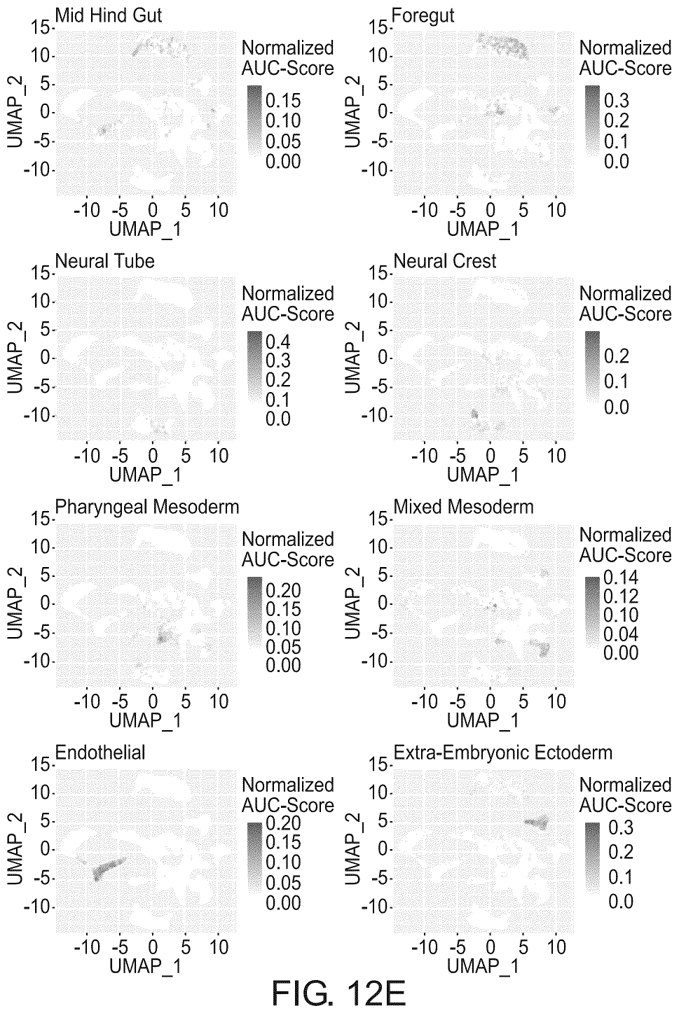

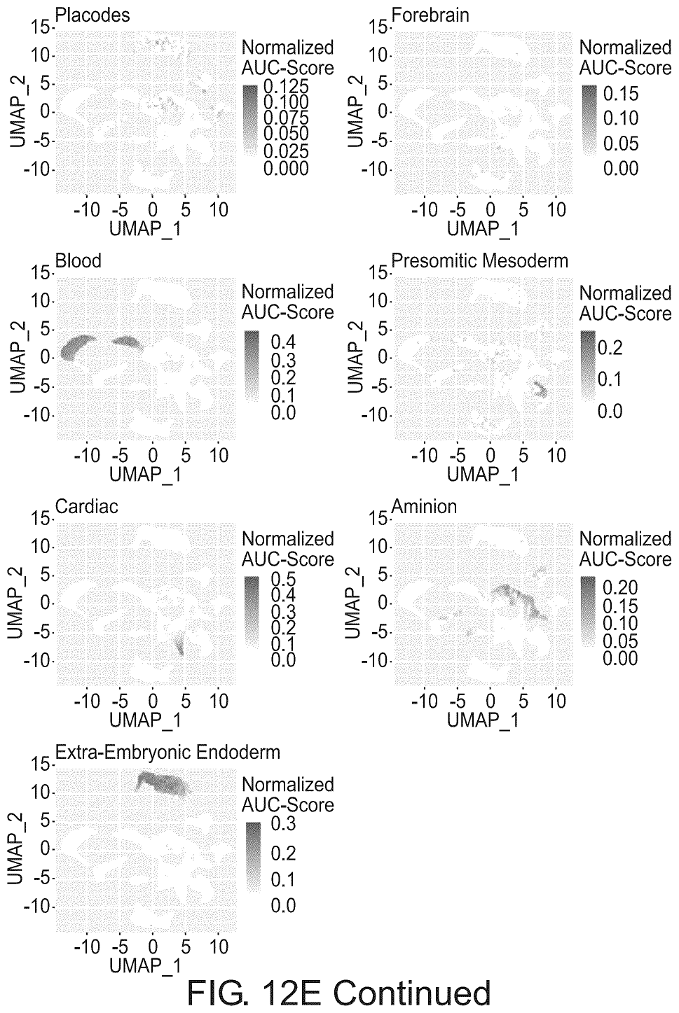

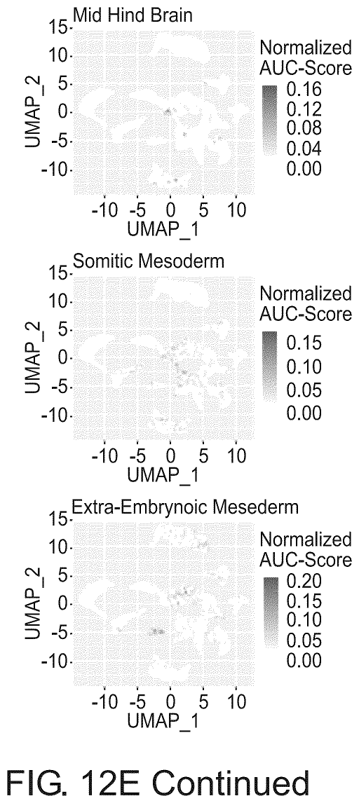

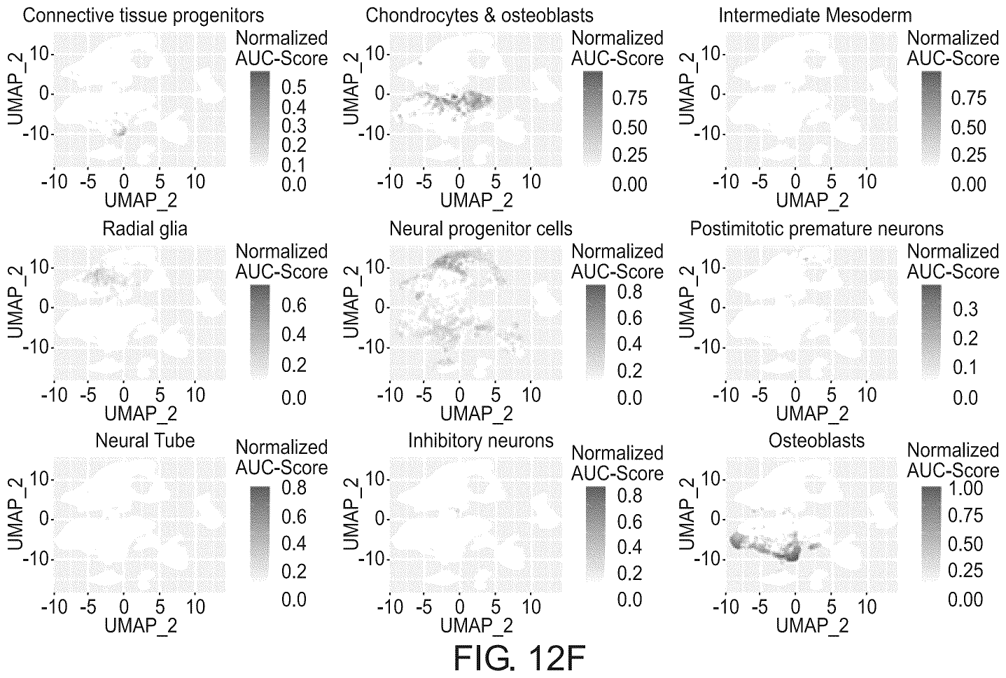

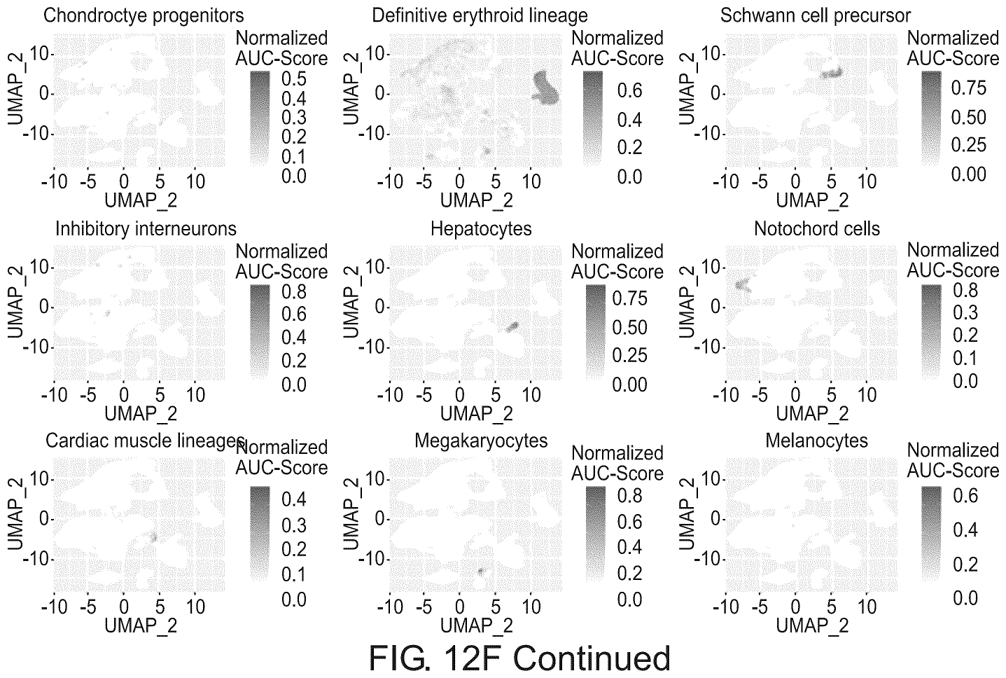

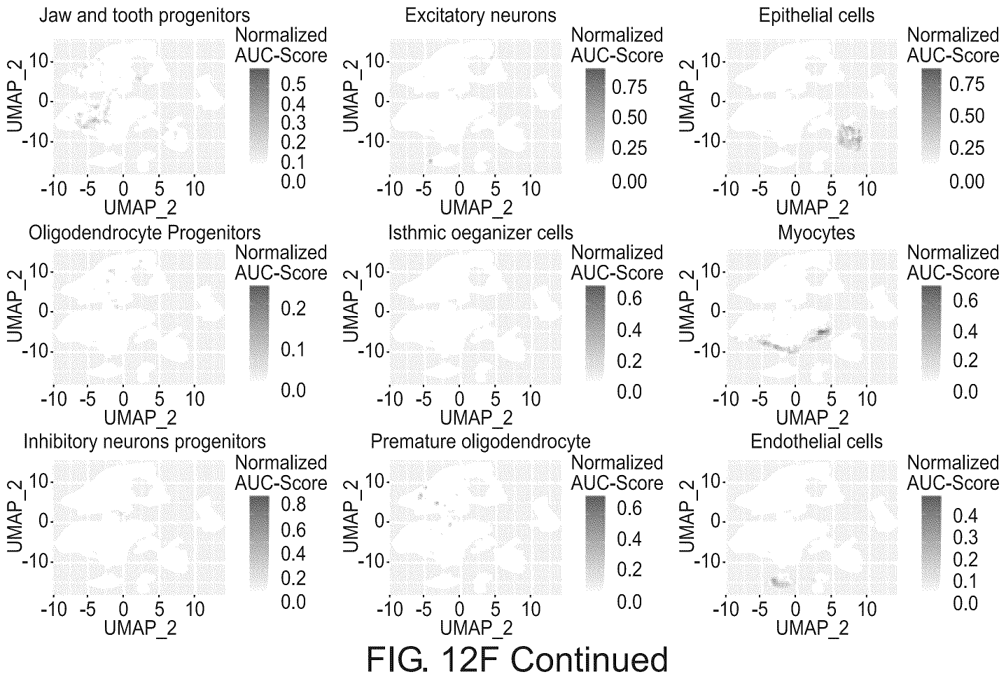

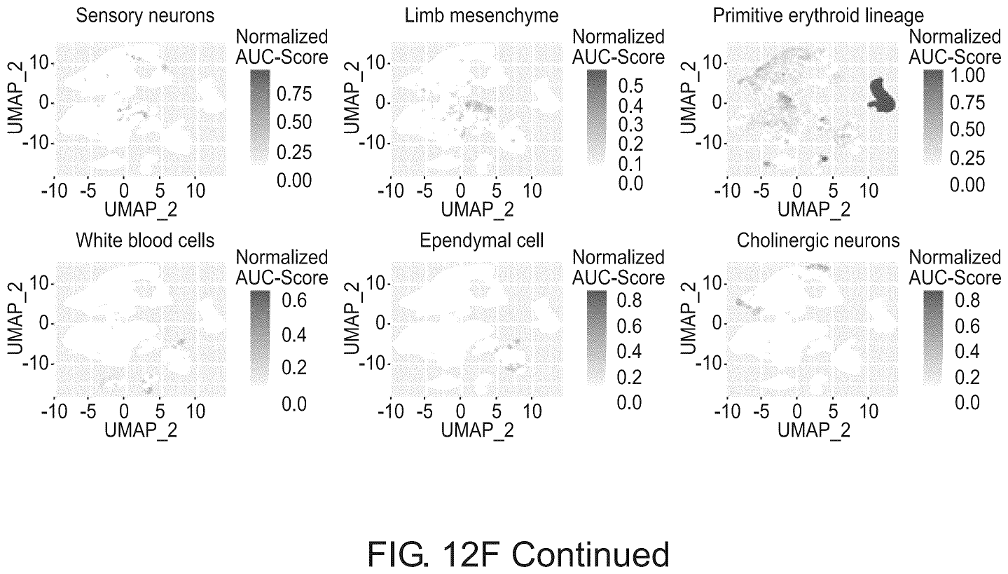

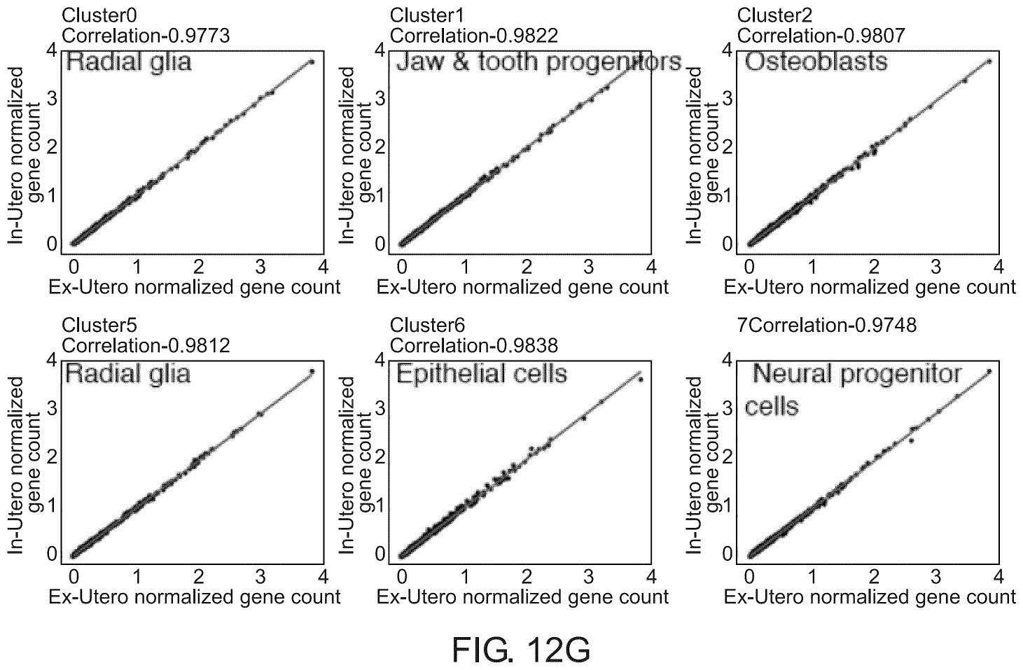

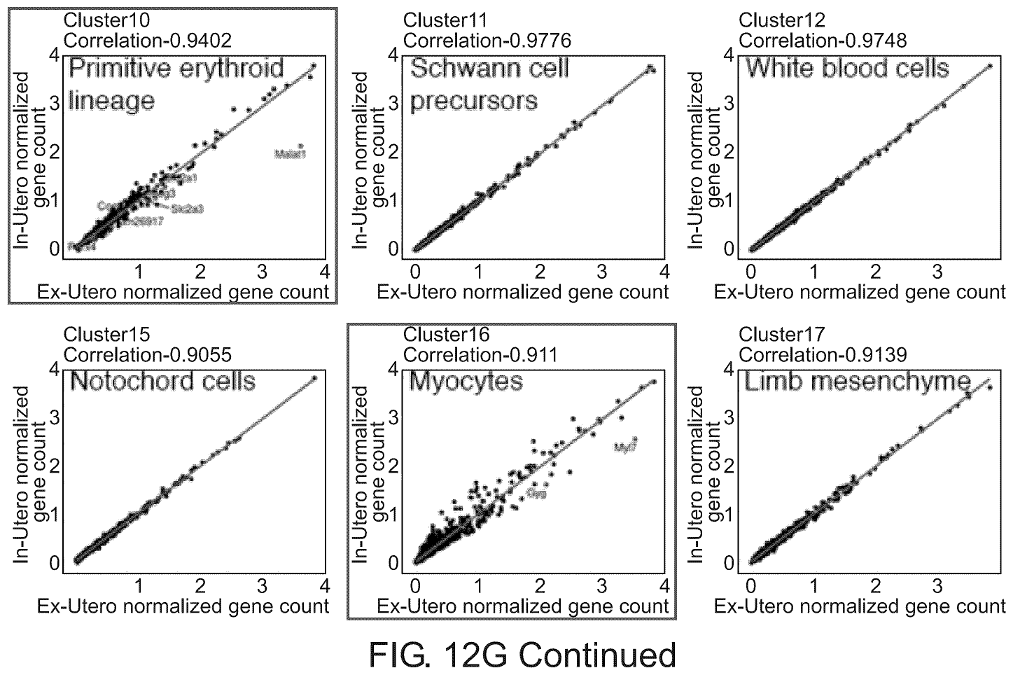

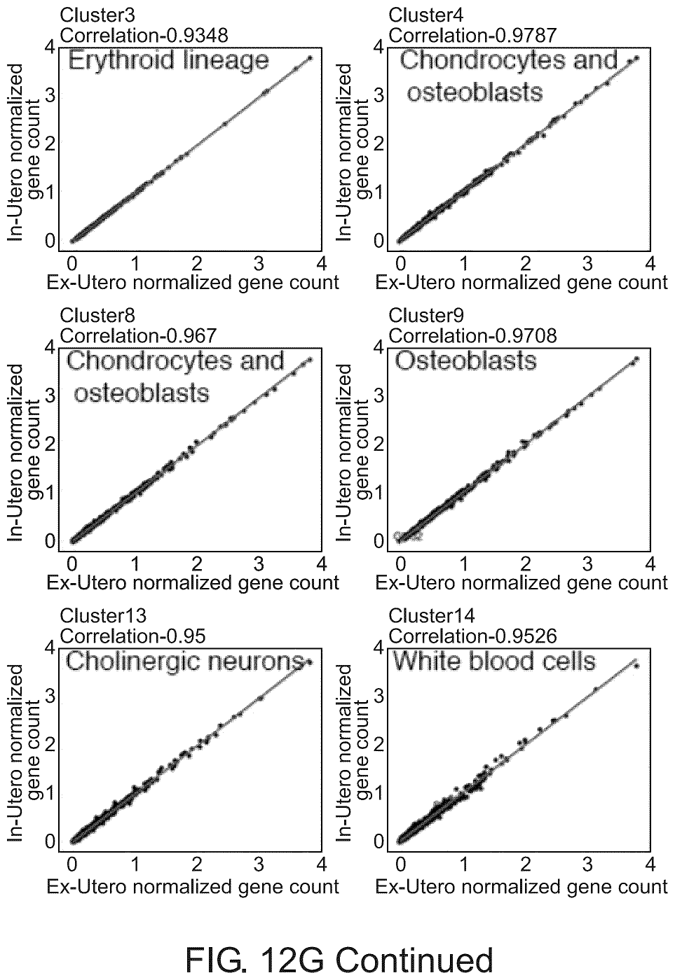

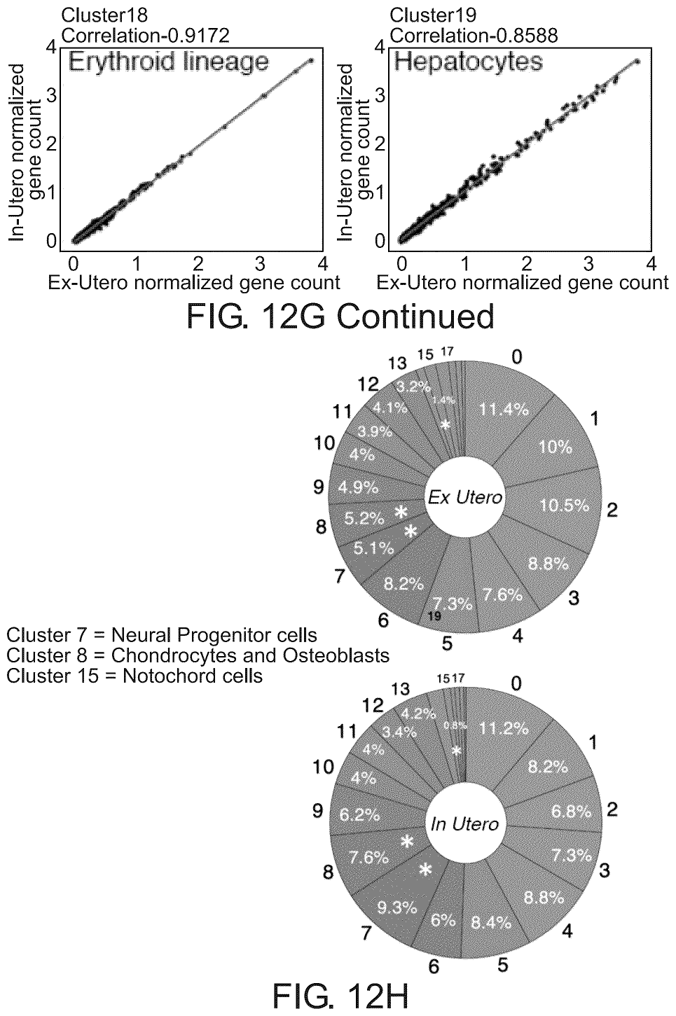

In order to characterize the various lineages present in the embryos, and to identify to which extent the global transcriptional profile of embryos developing ex utero mimics their in utero counterparts, a single cell RNA-sequencing (scRNA-seq) was performed on embryos grown ex utero for two days (E6.5+2 days) and was compared to cells obtained from equivalent embryos developing in utero (FIGS. 12A-B). Clustering analysis based on differentially expressed genes revealed 19 different cell states (FIG. 12C). The distribution of cell states was highly overlapping between in vivo and ex utero embryos (FIG. 2E). The identity of each cluster was annotated based on specific marker genes of the cell lineages previously defined by single-cell transcriptomics of early mouse embryos^{19,20} (FIGS. 12C and 12E). Derivatives of three germ layers as well as extraembryonic tissues were identified, and the profile of cell types found in embryos developing ex utero was equivalent to in utero (FIGS. 2E-F). In summary, the static conditions described herein faithfully recapitulate embryo development ex utero from the onset of gastrulation until somitogenesis (E6.5 to E8.5).

### Example 3

### Ex-Utero Culturing a Whole Mouse Embryo from E5.5 or E6.5 Towards Completed Organogenesis (E11 Onwards)

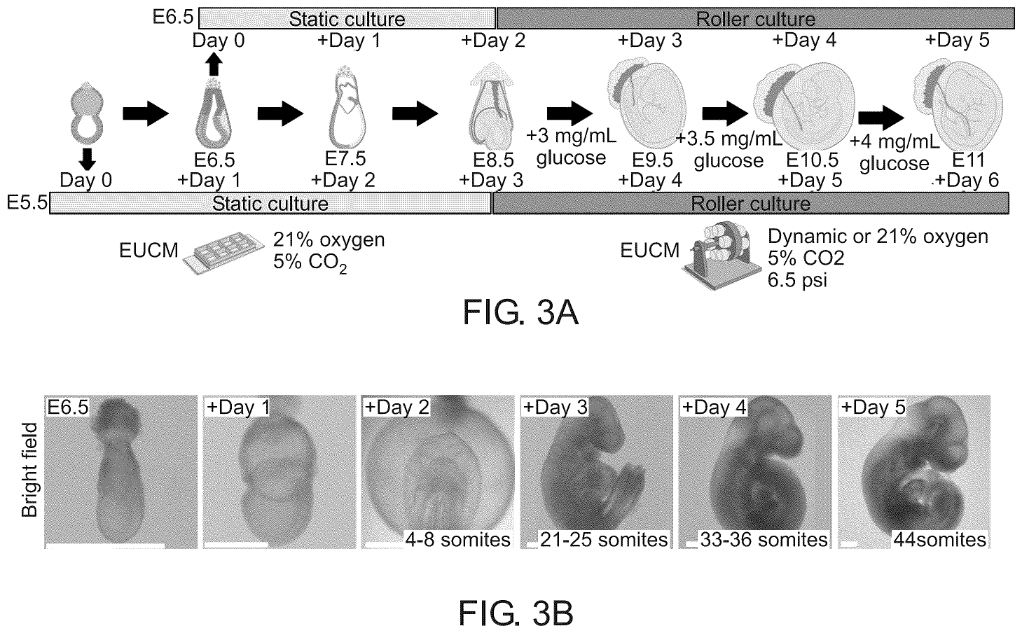

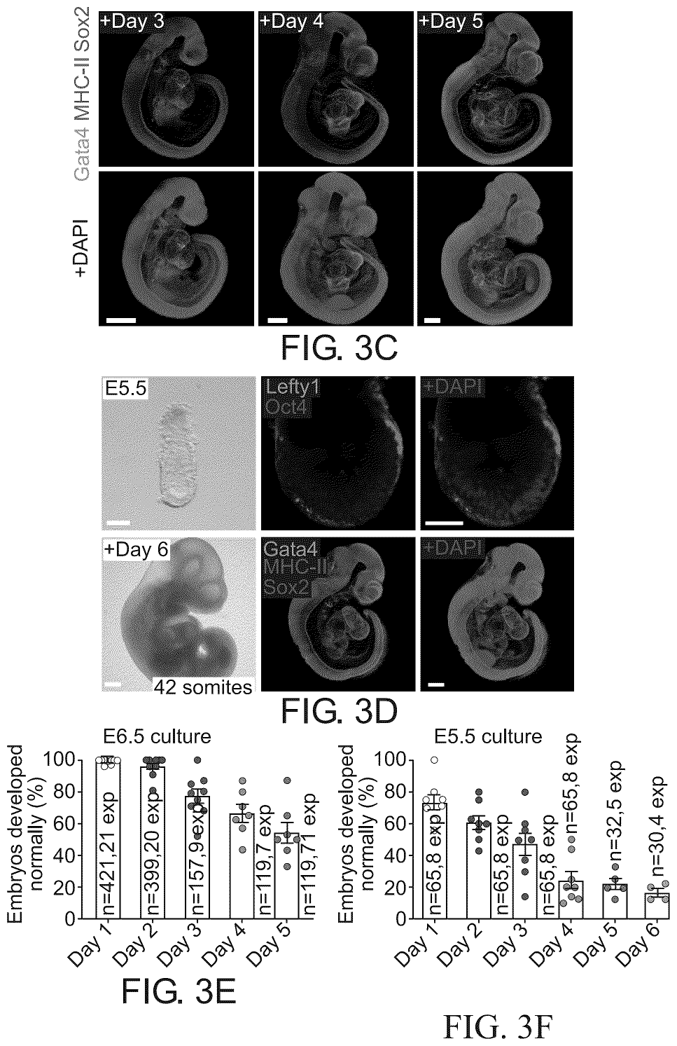

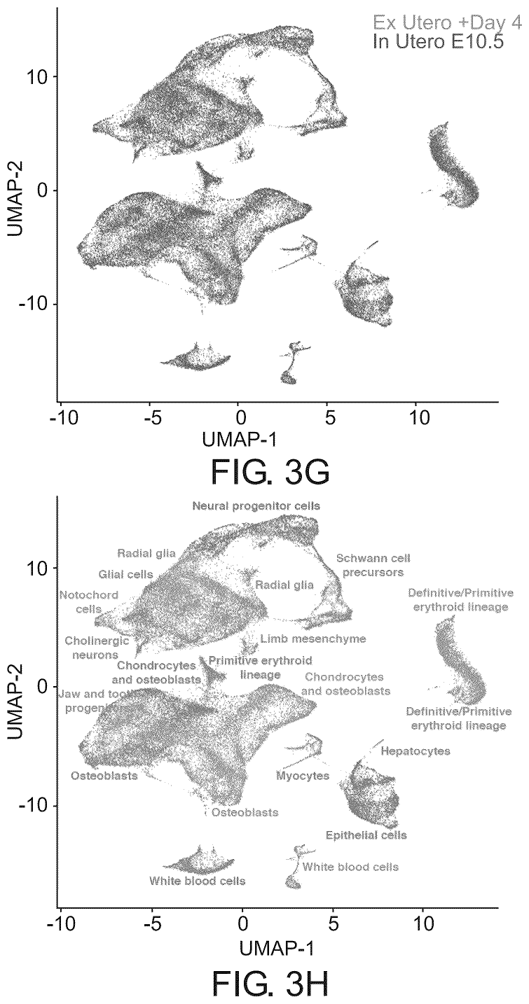

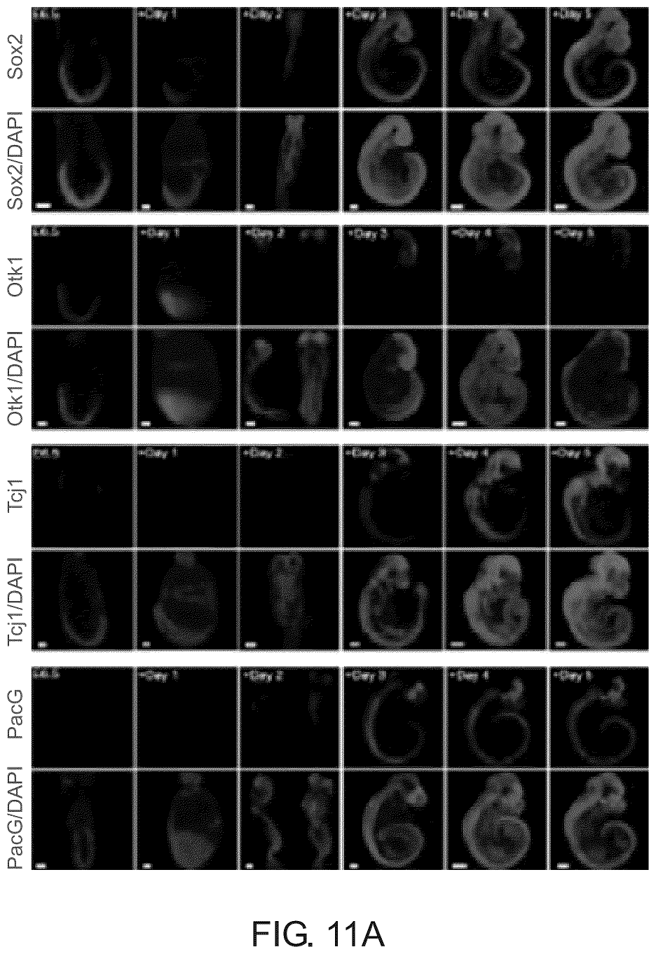

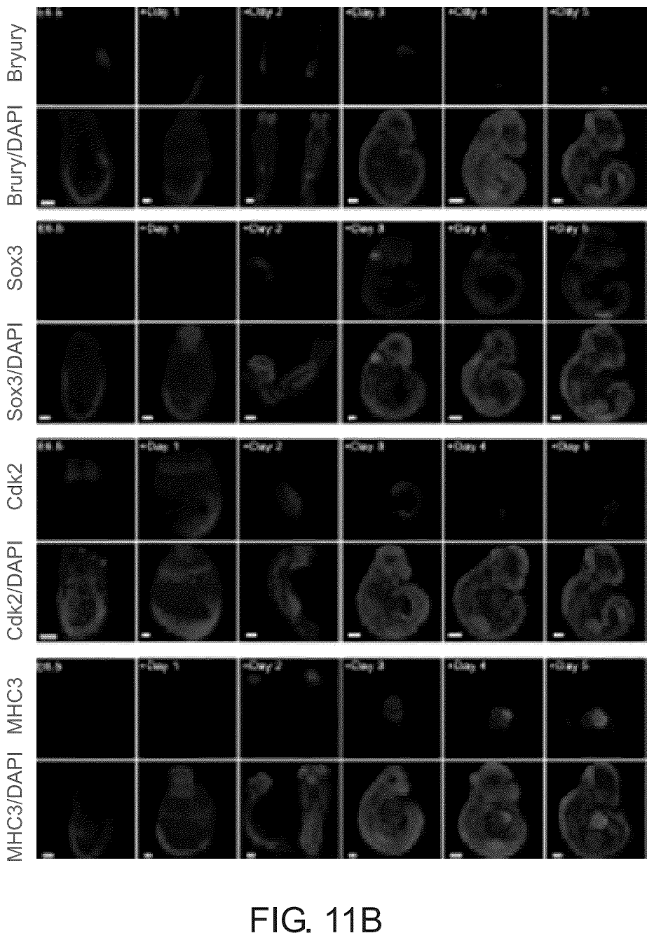

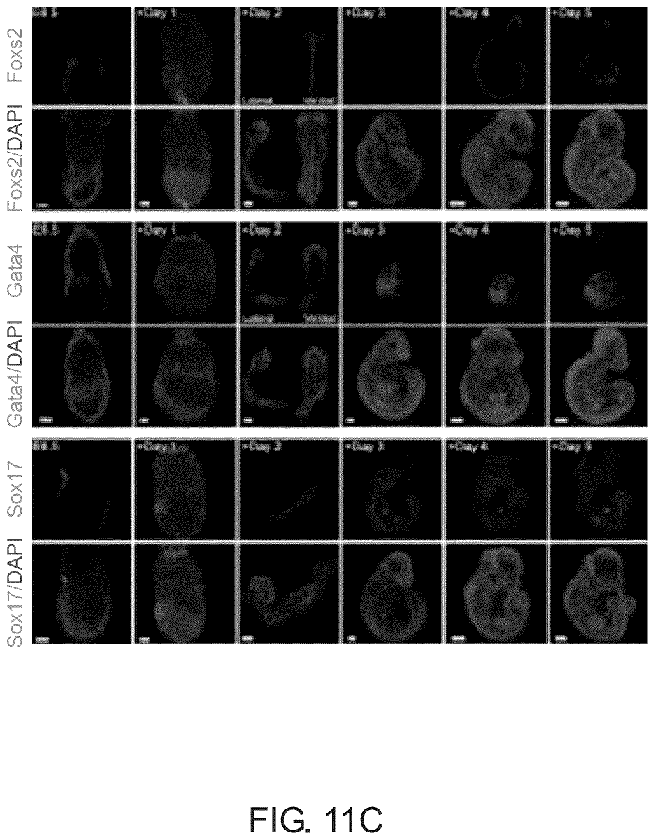

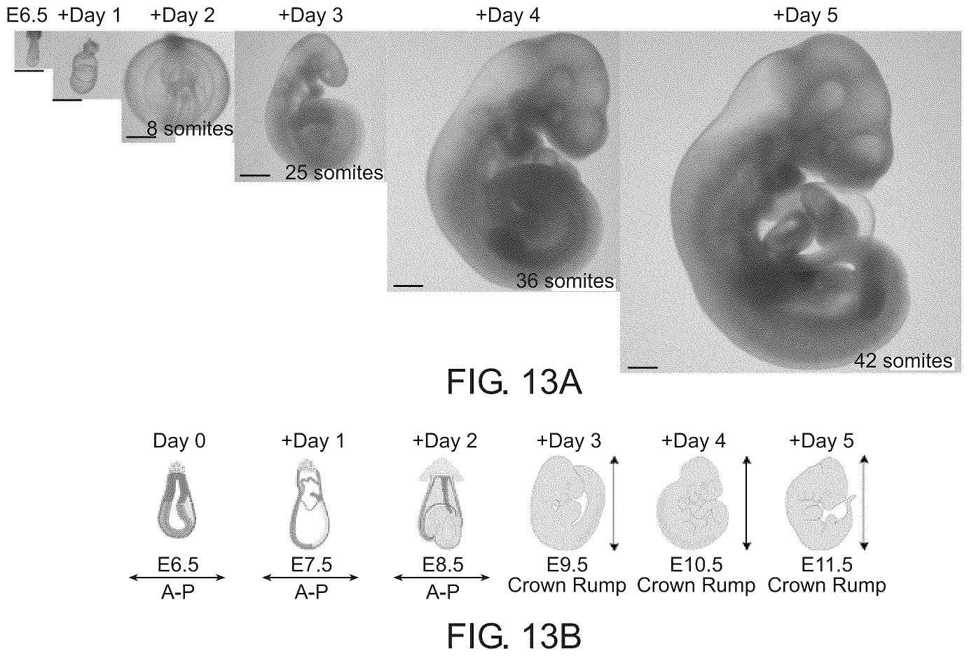

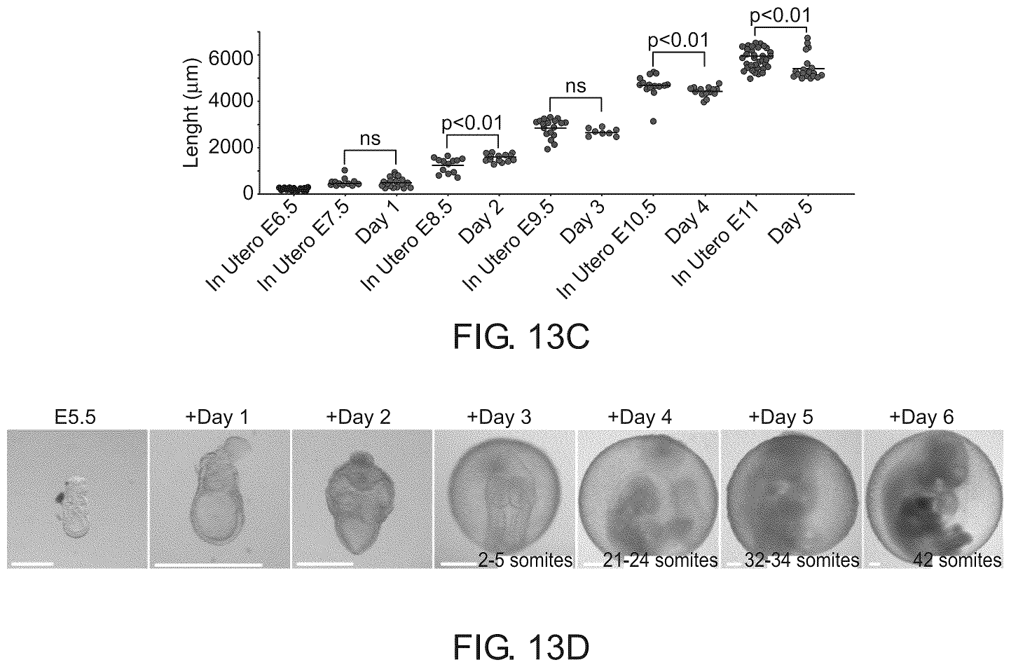

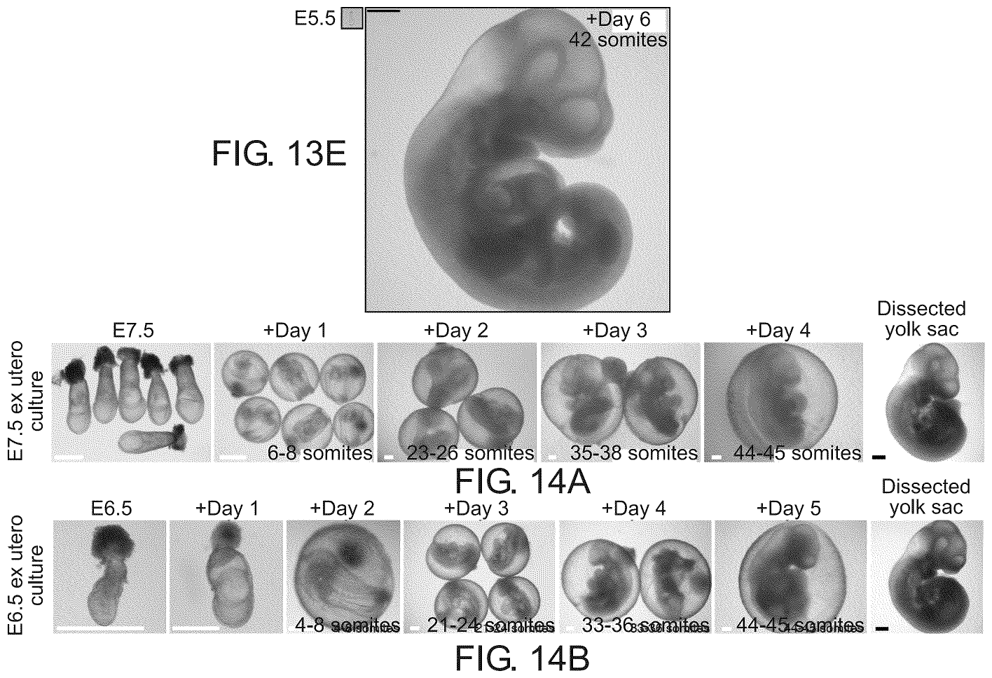

In the next step, the present inventors tested the ability to bridge mouse pre-gastrulation development to advanced organogenesis in culture by combining the developed static and roller culture protocols. To this end, following two days of static culture from E6.5 embryos, early somite-stage embryos (E8.5) were transferred into the roller culture protocol which allowed normal development of embryos isolated at early-streak stages until the hindlimb formation stage (E11), with 40% efficiency (FIGS. 3A-B and 10B). Only those embryos cultured in normoxia from E6.5 to E8.5 were able to continue growing after being moved to the roller culture system, which indicates that these conditions support appropriate embryo development (FIGS. 10A-B and 10G-H). Culturing the embryos in a constant atmosphere of 21% oxygen throughout the five days of culture increased the efficiency of development to 55% (FIGS. 3E and 10A). The same protocol and conditions were found competent to support pre-primitive streak E5.5 mouse embryo development for a total of 6 days ex utero, with 46% efficiency to reach E8.5 stage and ˜20% of the embryos to reach up to the 42 somites stage (FIGS. 3D, 3F and 13D-E). Even though a delay of ˜2 pairs of somites seems to arise in the timing of developmental events, embryo and tissue morphogenesis proceeded properly (FIGS. 3B and 13A). Comparable to in vivo development, the embryos increased in size from ˜200 μm at E6.5 to ˜5.4 mm at the 44 somites stage (FIGS. 13B-C). Immunofluorescence analyses of developmental genes confirmed that these markers are located according to their expected expression patterns (FIGS. 3C and 11A-C).

The transcriptional profile of cells isolated from embryos grown ex-utero and in utero matched-embryos was characterized by scRNA-seq (FIGS. 12A-B). The cells profiled were grouped into 20 different clusters described previously²¹ (FIGS. 12D and 12F). The annotated cell clusters identified represent mostly lineage-committed cell types comprising organs and tissues derived from all three germ layers, consistent with the advanced organogenesis stage of the embryos (FIG. 3H). Overall, the analysis confirmed that the composition of cell transcriptional states in the embryos developing ex utero until advanced organogenesis (E6.5+4 days) is equivalent to their in vivo counterparts (FIG. 3G). Comparison of the relative cell proportions across cell types showed no significant differences in the majority of clusters, while minor differences were found only in three clusters (FIG. 12H). Analysis of differentially expressed genes revealed a high correlation (˜0.9) between ex utero and in utero embryos for all cell states, with the most variable cluster showing only 0.4% (8 out of 2000) differentially expressed genes (FIG. 12G). The latter minimal difference in blood and cardiac gene expression signature at E10.5 (FIG. 12G) can be consistent with early signs of hydrops fetalis in the embryos. Collectively, these results demonstrate that embryos developing ex utero from pre-gastrulation stages, by a combination of static and rolling bottle cultures under the developed conditions, are capable of proper symmetry breaking, establishment of the germ layers as well as embryonic axis, and to subsequently differentiate and pattern tissues and organs without maternal interaction over a period of six days from symmetric pluripotent epiblast to advanced organogenesis stages.

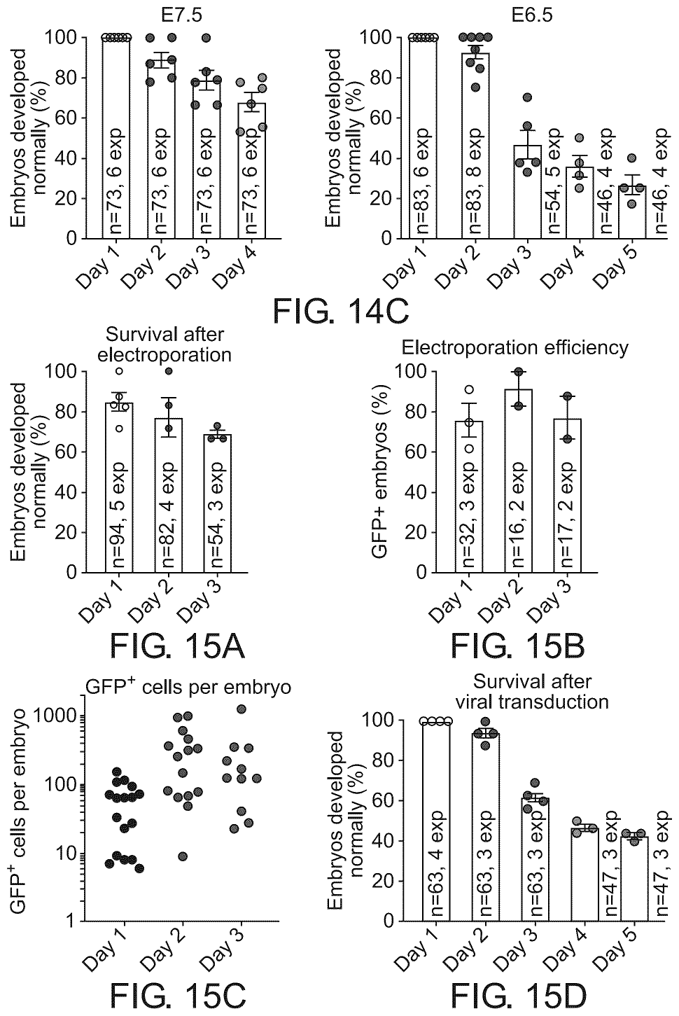

Notably, while optimizing the culture conditions it was found that HCS can be replaced with serum isolated from human adult blood (HBS) to allow ex utero development until the hindlimb formation stage (44 somites) starting from E6.5 and E7.5 (FIGS. 14A-C). Further, while E8.5 embryos were also obtained from E6.5 mouse embryos cultured in previously reported static conditions (50% DMEM/50% RS in 5% O₂) [McDole, K. et al. Cell 175, 859-876.e33 (2018)], they could not be developed further toward E9.5 upon transfer to the developed roller culture ex utero platform. The latter might be a result of minor, yet notable, morphological differences between the in vitro and in vivo embryos obtained in this protocol (FIG. 10O). Moreover, static culture does not sustain development of embryos beyond the early-somite stage (FIG. 10F). Increasing gas pressure in static conditions (2.5 and 5 psi), addition of 5% MATRIGEL® or supplementation with N2/B27 had a negative effect on embryo survival (FIGS. 10T-J and 10M-N). It was also noted that culturing embryos under hypoxic conditions drastically decreased efficiency and embryo quality after two days compared to 21% O₂ (FIG. 10G).

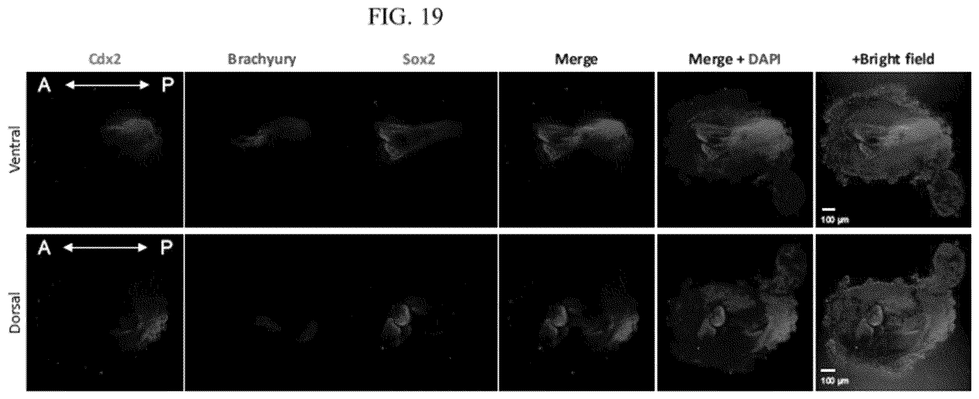

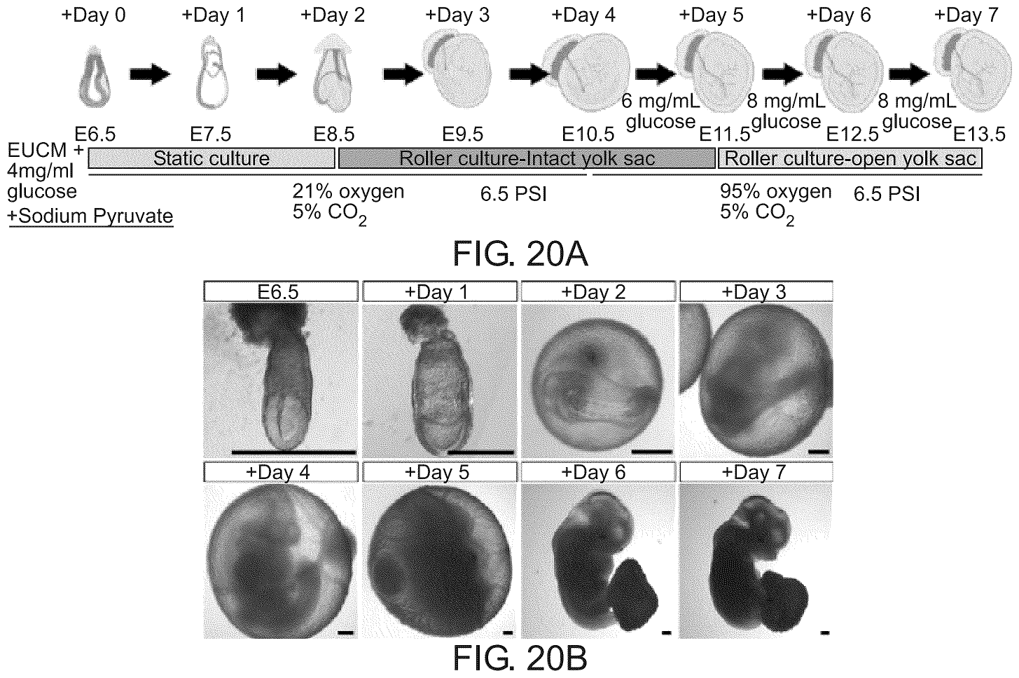

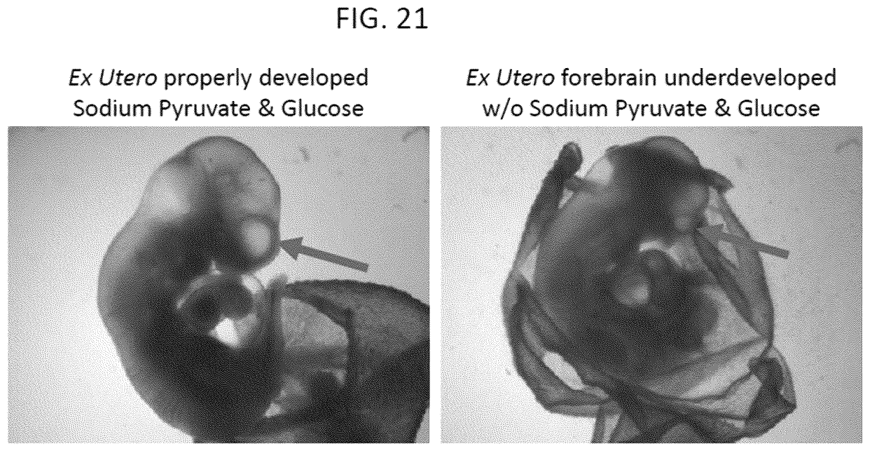

To further allow culturing of the embryos until E13.5, gas percentage in the roller culture was increased to 95% at E10.5 and at E11.5 the embryos were dissected out of the yolk sac and amnion, carefully avoiding rupture of any major yolk sac blood vessels, but keeping the yolk sac and umbilical cord attached to the embryo (FIGS. 20A-B). This procedure allows exposure of the body of the embryo directly to the oxygen and nutrients by opening the yolk sac, using the capillary circulation at the fetal surface for oxygen transfer. In addition, it was found that supplementation of the ex utero culture medium (EUCM) with additional 4 mg/ml of glucose and 1 mM Sodium Pyruvate since the beginning of the culture at E6.5 helps to improve culture efficiency (Table 1 hereinbelow and FIG. 21).

TABLE 1
Addition of sodium pyruvate to EUCM increases
efficiency of proper embryo development

| E6.5 | E7.5 | E8.5 | E9.5 | E10.5 | E11.5 | E12.5 |
|---|---|---|---|---|---|---|
| 8/8 | 8/8 - 100% | 7/7 (1 attached) | 6/7 - 85.7% | 6/7 - 85.7% | 4/7 - 57% | 4/7 - 57% |

### Example 4

### Manipulating a Whole Mouse Embryo Cultured Ex-Utero

One of the advantages that the developed ex utero culture platform offers is the ability to apply manipulations in post-implanted mouse embryos at the onset of organ formation, and follow their outcome on the same embryos following several days of further ex utero development.

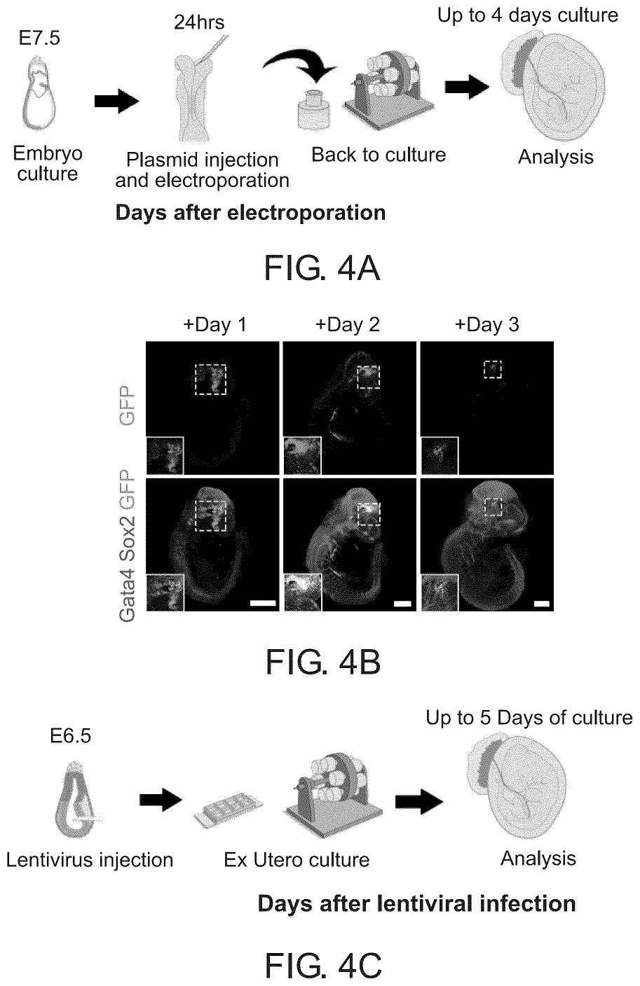

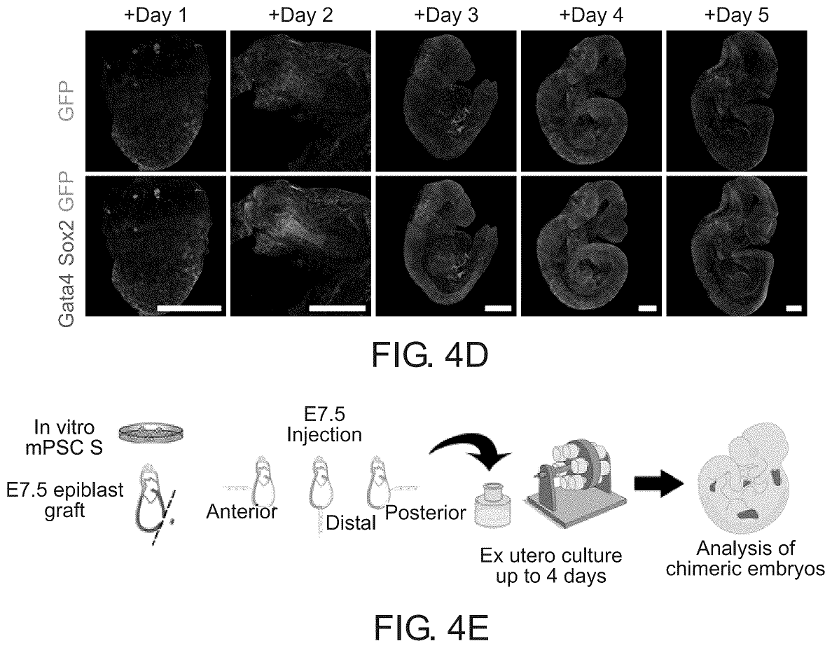

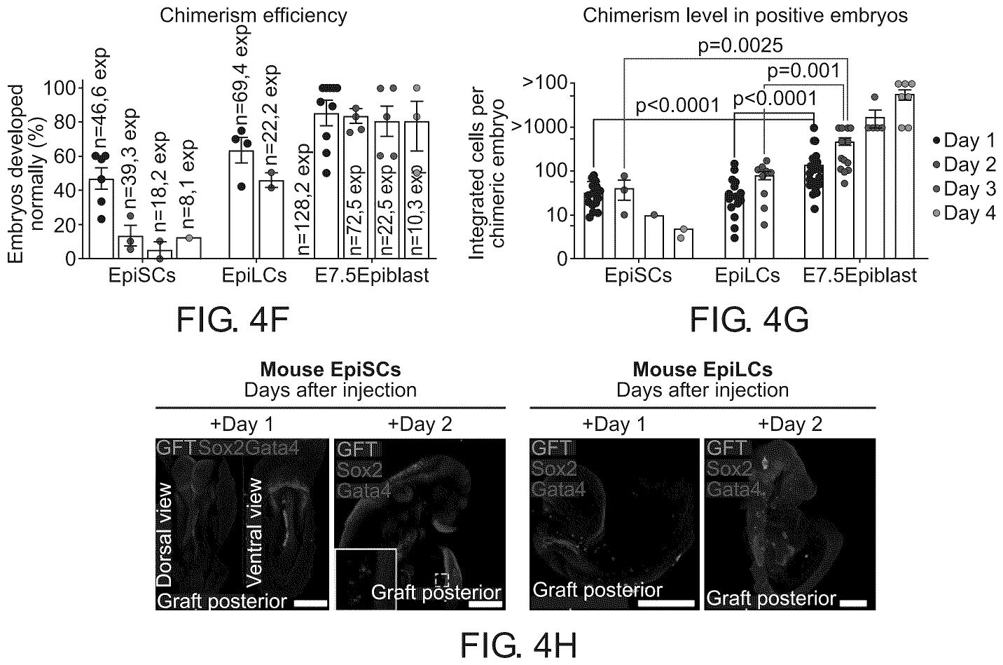

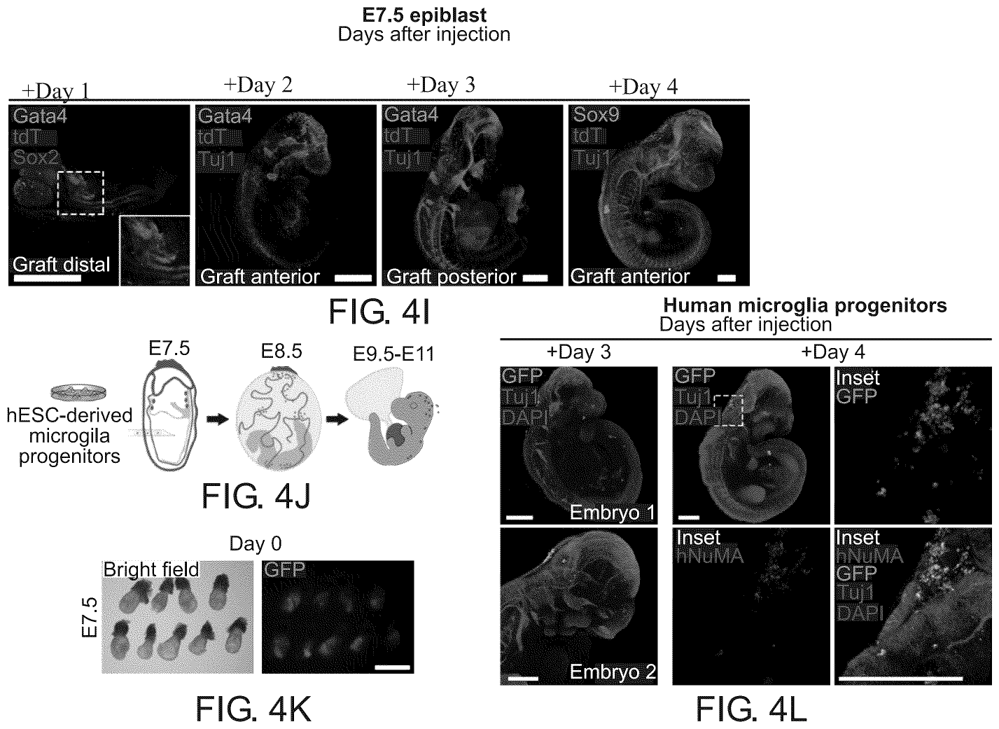

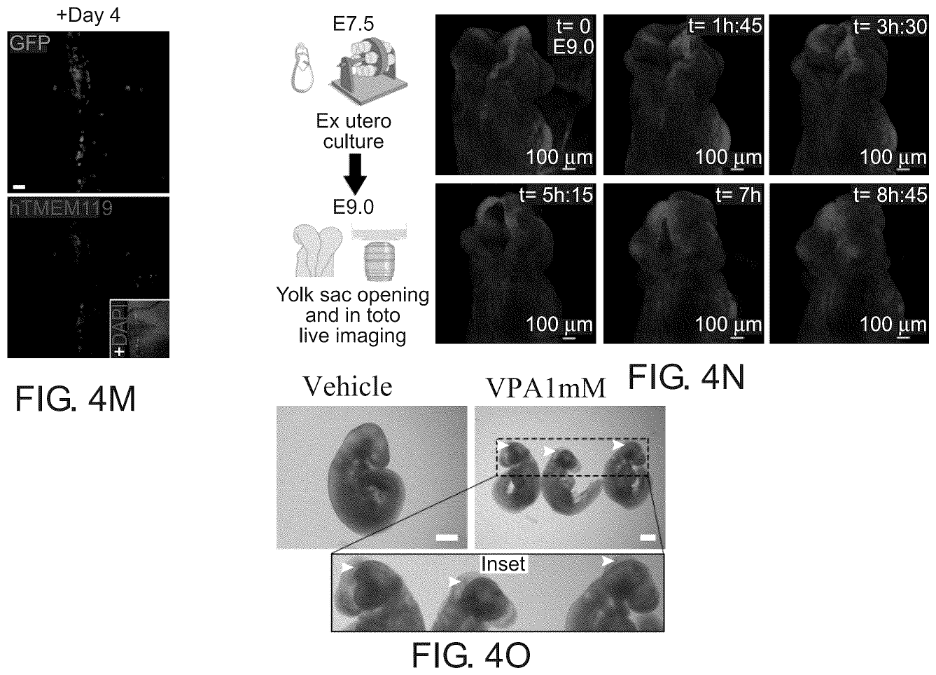

To this end, whole-embryo electroporation^{4,22} of a fluorescent genetic marker was performed at early E8.5 (prior to neural tube closure) followed by long-term ex utero culture (72 hours). Specifically, embryos at E7.5 were dissected and cultured for 24 hours. Afterwards, a GFP plasmid vector was injected into the neural tube and electroporated to label a population of neural cells. Electroporated embryos were then put back in culture for up to three days (FIG. 4A). Around 68% of the embryos developed properly until the hindlimb stage following electroporation (FIG. 15A). Cells expressing the GFP plasmid were widely distributed in the neural tissues following 1-3 days of culture in 75% of the embryos (FIGS. 4B and 15B-C).

The ability to perform genetic modifications by lentiviral transduction²³ was also shown in E6.5 embryos by microinjecting lentiviral vectors harboring an EGFP gene (FIG. 4C). Lentiviral transduction yielded an embryo survival rate similar to controls and did not affect morphology or tissue differentiation (FIGS. 4D and 15D). After 24 hours, GFP was detected throughout the epiblast and extraembryonic tissues, and by the last culture day, GFP expression was extensively spread over the embryo and yolk sac in >90% of the embryos (FIGS. 4D and 15E).

Following, the ex utero culture platform was harnessed to analyze chimeric mouse embryos obtained after microinjection of primed pluripotent stem cells (PSCs) at post-implantation stages²⁴. Evaluating the chimeric potential of primed mouse PSCs upon microinjection has been limited by the lack of protocols that enable transfer of post-implantation embryos in utero. Clusters of GFP-labeled mouse epiblast stem cells (EpiSCs) or epiblast-like stem cells (EpiLCs) were microinjected into the anterior, distal or posterior epiblast of E7.5 embryos, which were subsequently cultured ex utero (FIGS. 4E and 15F-J). Following 24 hours, 50-60% of chimerism efficiency was observed for both EpiSCs (27/49 embryos) and EpiLCs (44/69 embryos) injected in the posterior epiblast, with an estimated number of transplant-derived cells ranging between 10 to 100 cells distributed along the embryo body axis (FIGS. 4F-G), consistent with previous studies²⁵⁻²⁷. Co-immunostaining for Sox2 and Gata4 confirmed that the cells integrated into embryonic tissues (FIGS. 4H and 15I). However, the number of EpiSCs-derived GFP⁺ cells decreased over subsequent 3 days and were outcompeted by the cells of the host (FIGS. 4F-G). Low integration was also evident when microinjecting EpiSCs and EpiLCs in the anterior epiblast (FIG. 15J). Isogenic naïve ESCs microinjected into blastocysts that were subsequently transferred and re-isolated at E7.5 and subjected to ex utero cultures, yielded high contribution chimeras (FIG. 15H). The latter exclude genetic background or ex utero culture systems as the underlying cause for limited chimeric integration of in vitro derived primed PSCs. Lastly, primed cell clusters isolated directly from tdTomato⁺ E7.5 embryonic epiblasts were injected into recipient embryos (FIG. 4E). Unlike EpiSCs or EpiLCs, in vivo derived E7.5 epiblast orthotopic grafts contributed extensively and adequately to chimeric embryos across different tissues (more than 10,000 integrated cells at the last day of culture) (FIGS. 4I and 15K). These results suggest that, in relation to their in vivo counterparts, in vitro primed PSCs possess a limited capacity to expand and significantly incorporate into host tissues even when they are injected into developmentally matched post-implantation stages and allowed to undergo advanced organogenesis ex utero.

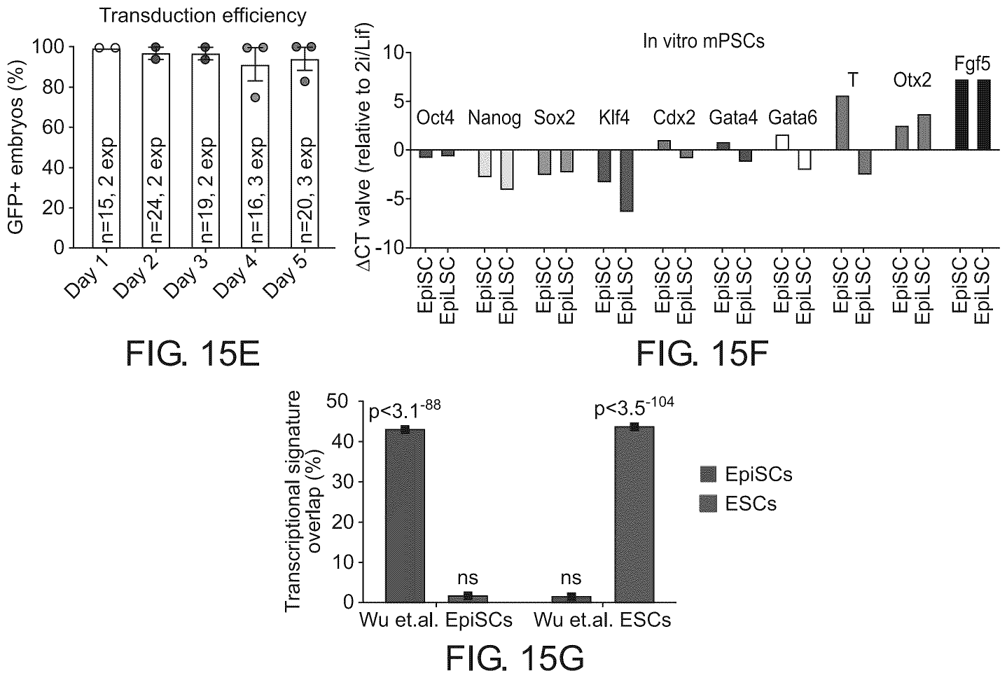

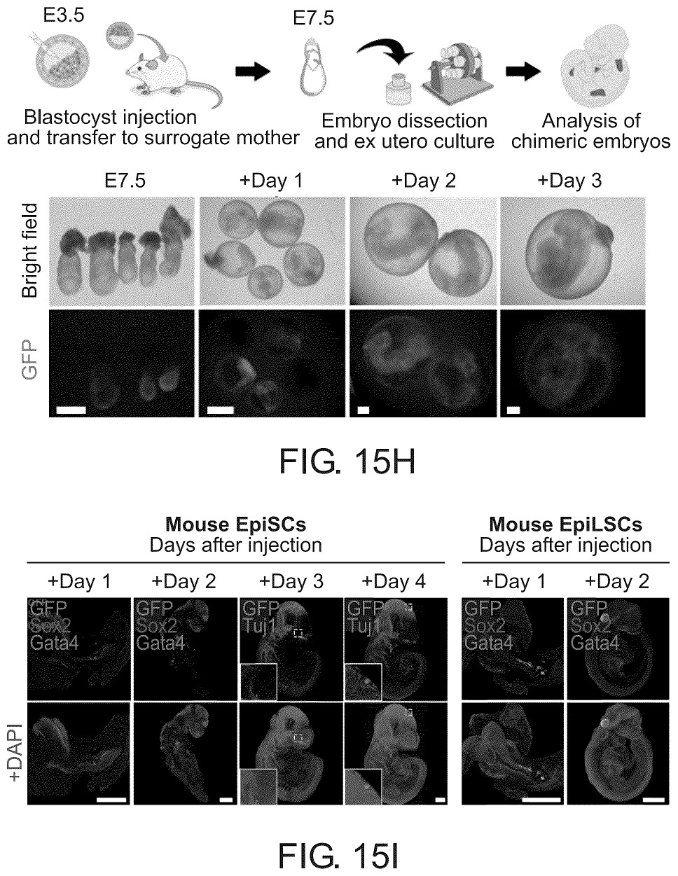

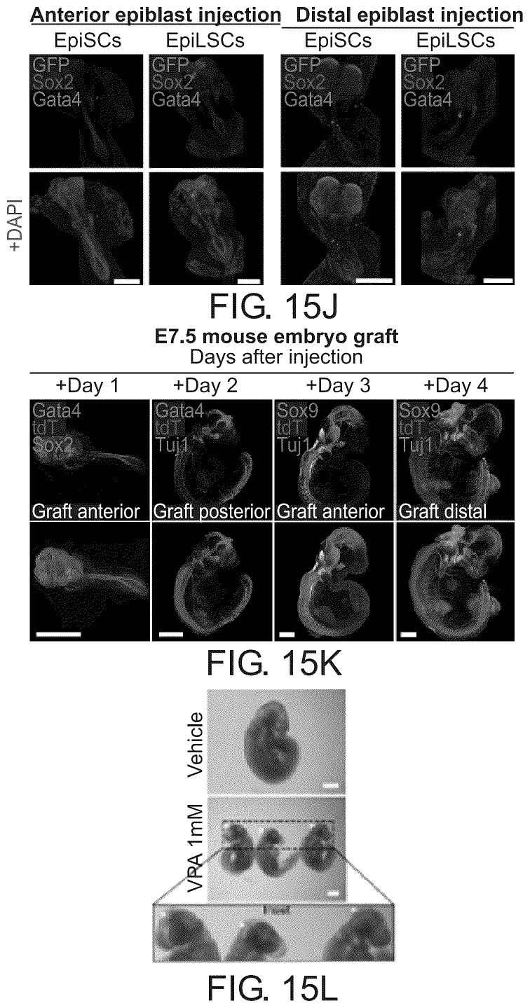

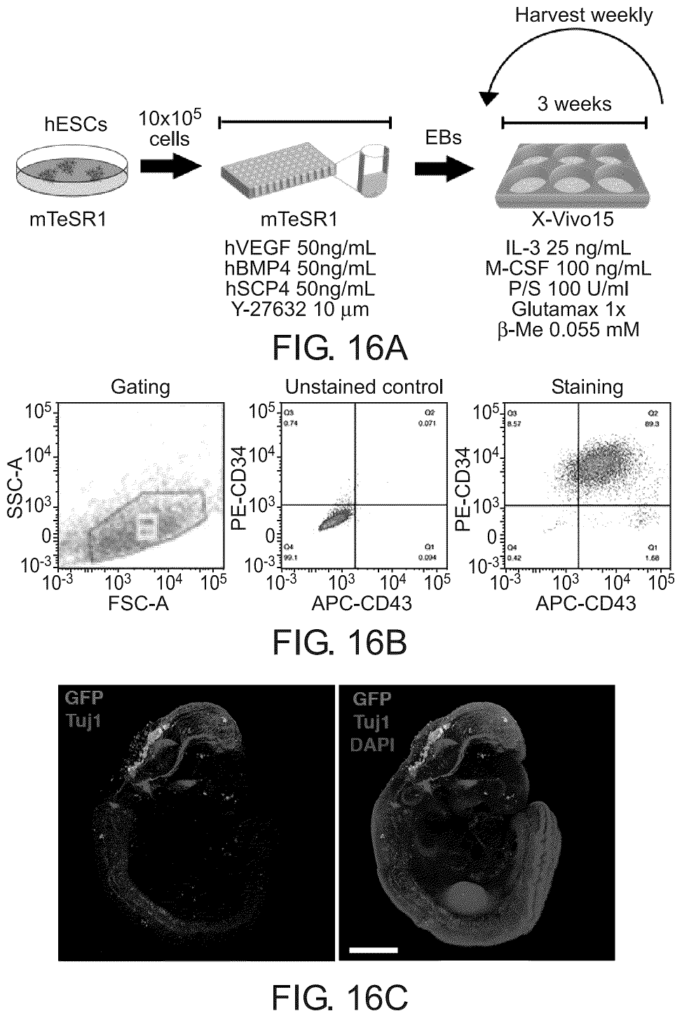

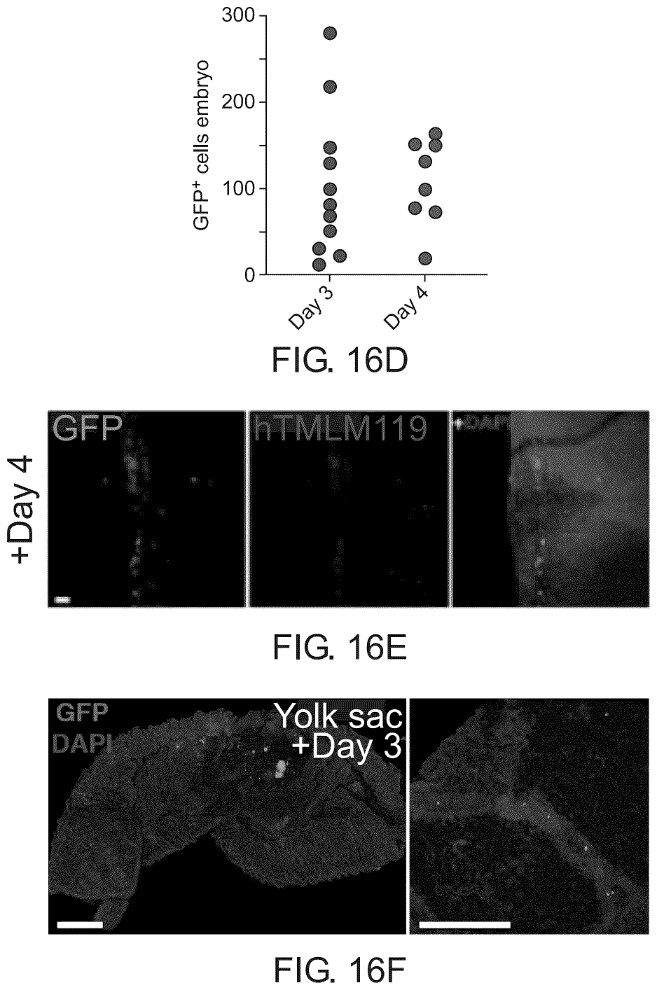

In the next step, GFP-labeled primitive microglia progenitors were derived from human PSCs²⁸ (FIGS. 16A-B), and microinjected into mouse embryos at E7.5 followed by ex utero culture (FIGS. 4J and 4K). Analysis of integrated human cells revealed that microglia precursors robustly integrated, proliferated and migrated into the host brain (FIGS. 4I and 16C-D). The microglial identity of the injected cells was confirmed by the presence of double positive cells for GFP and TMEM119 (FIG. 16E). GFP⁺ human cells were also detected circulating through the yolk sac and yolk sac vessels, indicating that human microglia progenitors can migrate through the mouse embryonic circulation (FIG. 16F). These results demonstrate the usability of the platform described herein to shed light on development of human cells in the context of cross-species embryonic chimeras²⁹.

In addition, in toto confocal live imaging can be applied for the ex utero developed embryos. As an example, imaging of neural tube closure in tdTomato⁺ mouse embryos, which were maintained ex utero since E7.5 and subsequently mounted for live confocal imaging at E9.0, was applied to visualize the dynamics of convergence and closure of the neural folds for ˜9 hours (FIG. 4M). Further, the teratogenic effect of different drugs can be tested by the developed ex utero culturing protocol. For example, the teratogenic effects of valproic acid on neural tube closure could be recapitulated by supplying this teratogen to the embryo environment ex utero (FIG. 15I).

### Example 5

### Ex-Utero Culturing a Whole Mouse Embryo from E0 to Late Organogenesis

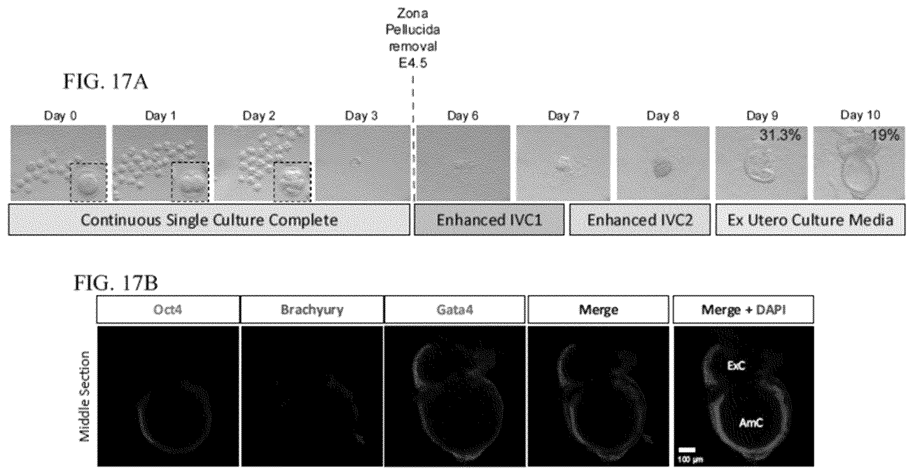

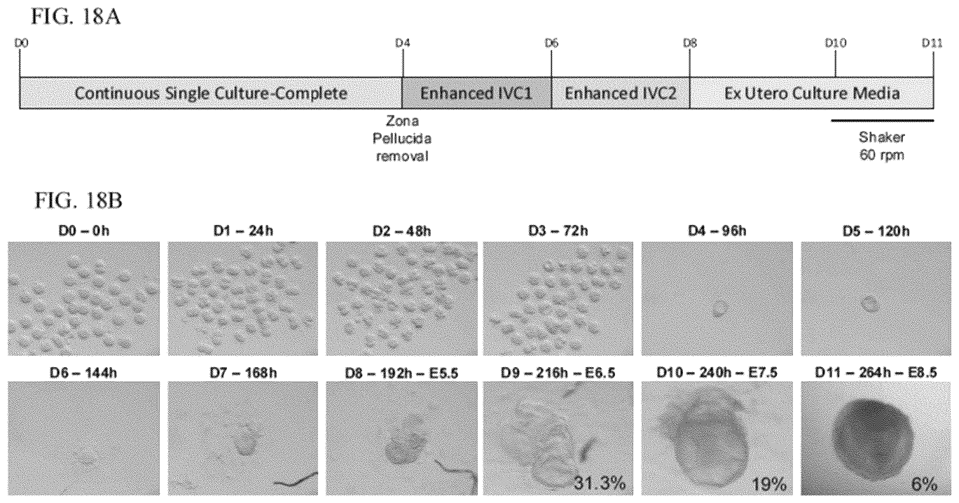

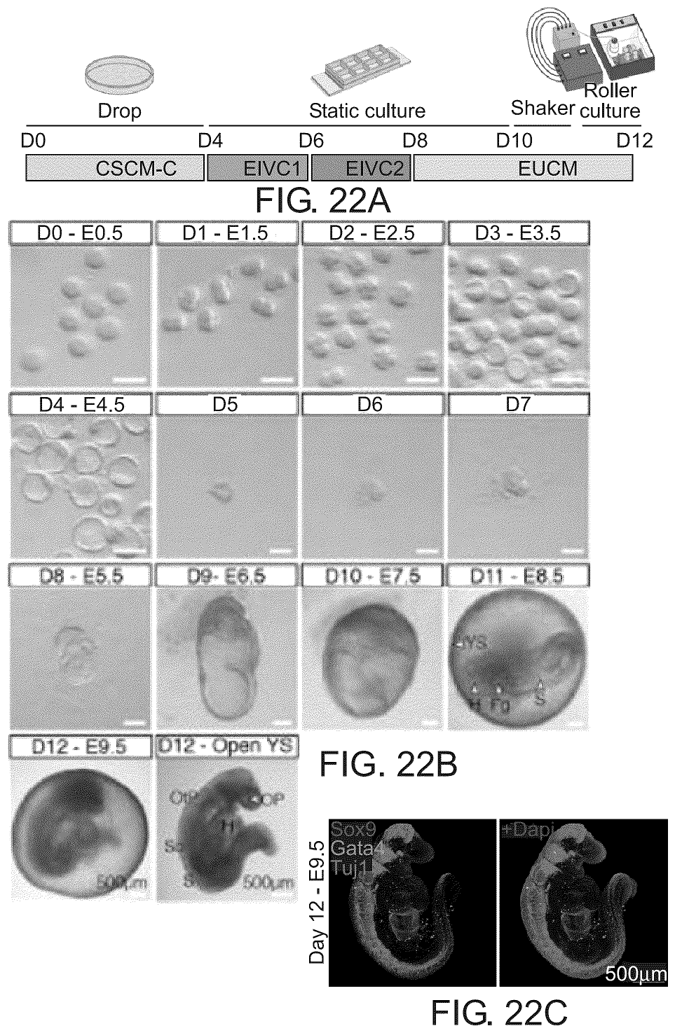

In the following step, the present inventors aimed to expand the ex utero culture protocol by finding conditions to grow the mouse embryo from a single fertilized egg. To this end, a culture system was established that enabled growing a zygote mouse embryo, through implantation, gastrulation and early somitogenesis stages. According to this protocol, embryos are grown through pre-implantation development using the Continuous Single Culture Complete media (CSCM) or KSOM media; followed by culturing in a combination of two media (Enhanced In Vitro Culture media 1 (EIVC1) and 2 (EIVC2)] across the implantation period until the early egg cylinder stage; and finally culturing the egg cylinders until advanced gastrulation using the Ex Utero Culture Media (EUCM) (FIG. 17A). Transfer of the embryos to a rotating culture on a shaker for 24 hours placed in a conventional tissue culture incubator allows further development to the early somite stage (FIG. 18A). Of note, culturing the embryos through the implantation period until the early egg cylinder stage resulted in a 2 days delay in development in comparison to an in utero counterpart. The embryos cultured ex utero according to this protocol developed properly, as shown by analyzing the distribution of cell types and the expression of lineage markers (FIGS. 18B and 19). Transfer of the embryos at the early somite stage to the roller culture system coupled to the gas regulation module allows further development to E9.5 (FIGS. 22A-C).

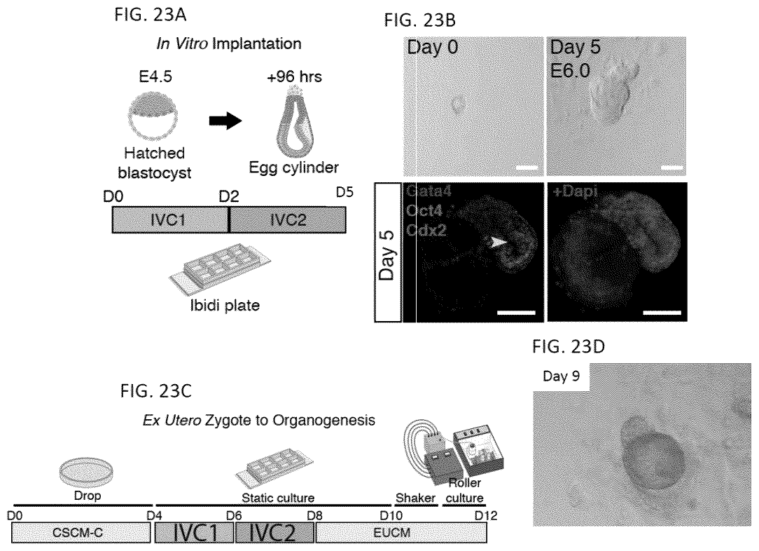

Remarkably, culturing mouse blastocysts in IVC1 and IVC2 media described by Bedzhov et al. (Cell 2014, PMID: 24529478) using their established protocol (FIGS. 23A-B) did not yield gastrulation or organogenesis at the end of the protocol, while normal gastrulation and advanced organogenesis were obtained with modified and new protocol shown in FIG. 22A.

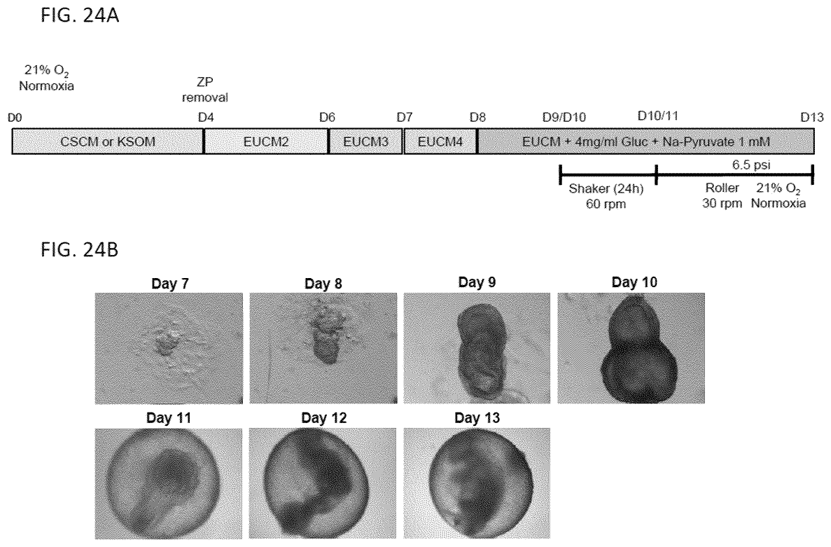

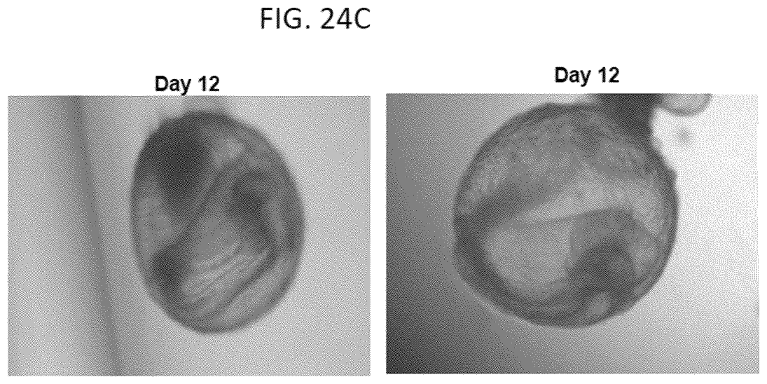

In the next step, the inventors were able to replace EIVC1 and EIVC2 in a combination of EUCM2, EUCM3 and EUCM4 (FIGS. 24A-B and the materials and methods section hereinabove). Moreover, supplementing EUCM2/3/4 with NEAA, D-Glucose, ITS-X, β-Estradiol, Progesterone and N-acetyl L-Cysteine can enhance efficiency of proper embryo development and the quality of obtained embryos (FIG. 24C).

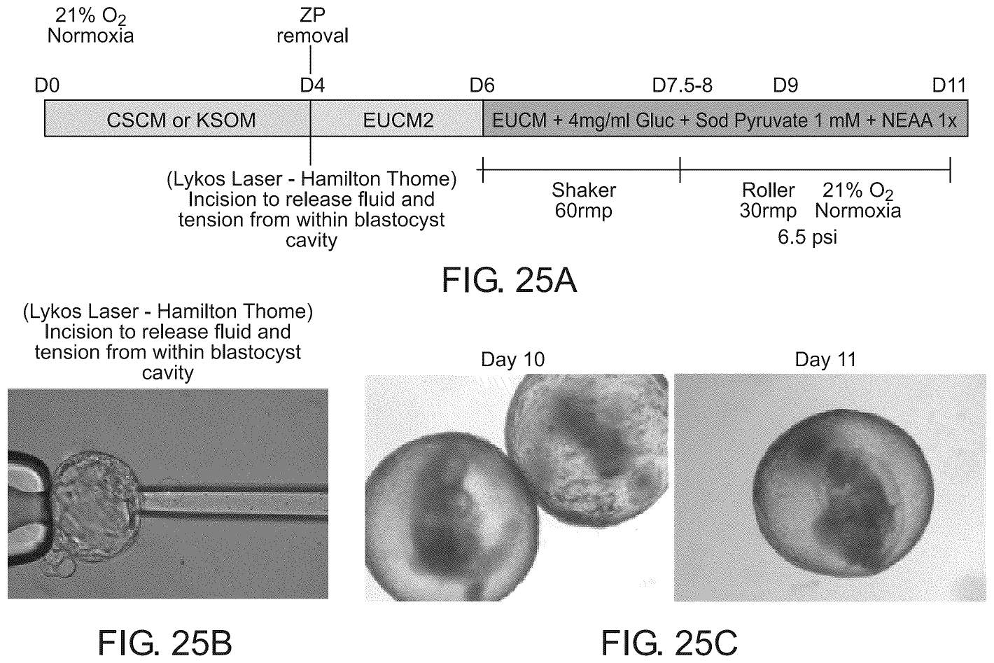

As noted above, culturing the embryos through the implantation period until the early egg cylinder stage resulted in a 2 days delay in development in comparison to an in utero counterpart. To overcome this delay, an alternative protocol utilized a Lykos Laser system by Hamilton Thorne in order to excise the mural trophectoderm of the blastocyst as previously described (PMID: 32944901) leading to release of intra-blastocyst fluid and pressure prior to culturing (FIGS. 25A-C).

### Example 6

### Manipulating a Whole Mouse Embryo-Tetraploid Embryo Complementation Microinjection

Tetraploid embryo complementation microinjection approach is a novel mouse engineering approach, in which 2-cell mouse embryos are electro fused and then continue to develop until the blastocyst stage (PMCID: PMC5905676). The mouse ESCs/iPSCs microinjected into these 4n host blastocysts, can generate unique “all ESC/iPSC” chimeras after in utero transfer (PMCID: PMC5905676). In these embryos the host tetraploid 4n blastocyst cells can generate only extra-embryonic tissues, while the embryo proper will be composed 100% from the injected ESCs/iPSCs. The latter prove unequivocally, that when using host embryos as “carriers”, in vitro cultured PSCs can make entire embryos under the right experimental settings.

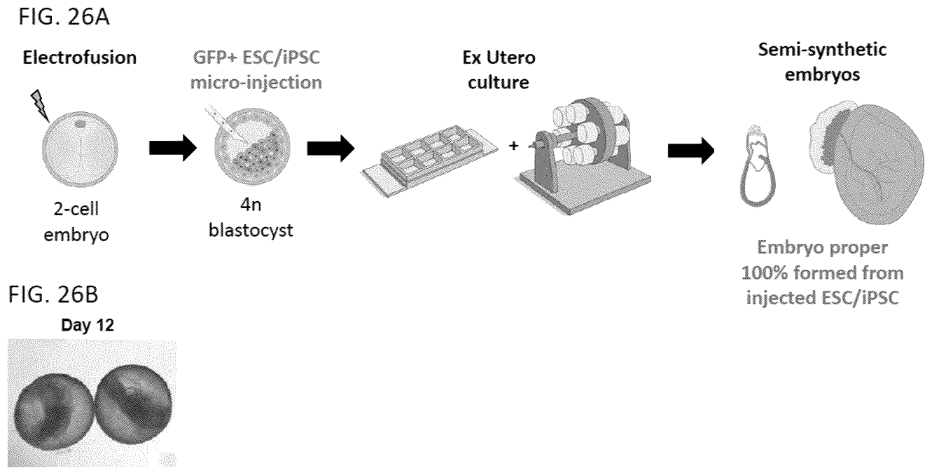

To this end, EGFP-labeled in vitro expanded iPSCs/ESCs are microinjected in tetraploid host blastocysts and the generated embryos are further cultured ex-utero by the methods described herein. In the generated embryos the extraembryonic tissue originates only from the tetraploid host blastocysts and the embryo is formed only from the injected in vitro generated GFP+ PSCs (FIGS. 26A-B). This platform can be also used for direct testing of embryonic phenotypes ex utero by going directly from mutant ESCs/iPSCs.

### Example 7

### Manipulating a Whole Mouse Embryo-Mutant Embryos with Restricted Developmental Potential

The aim in this Example was to implement and optimize a robust knock-out system to perturb embryos at e.g. E6.5 and subsequently grow them in an ex-utero system until early organogenesis. This can used for example to knockout a selected gene to selectively perturb a certain organ, thus making the embryo with limited developmental potential and not being able to sustain viability. In this scenario, other organs develop normally and can be used for further applications. This may be used to resolve ethical problems.

For example, making a headless or heartless embryo, is more palatable to ethical committees and for future applications. Heart beat is considered by many to represent a living entity. Thus, deletion of e.g. Mesp1 or NKX2-5 genes allows normal embryo development without the formation of the heart. Alternatively, deletion of e.g. the Lim1 gene allows normal embryo development without formation of the head (PMID: 7700351).

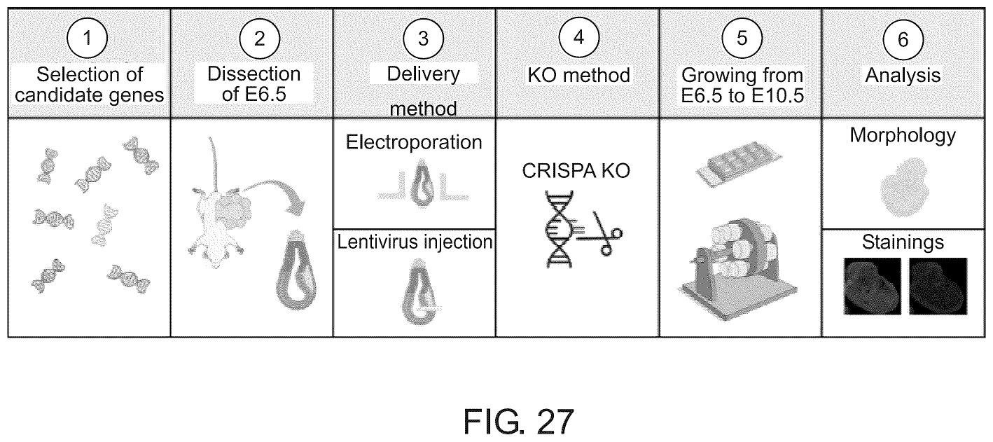

To this end, knocking out the targets is performed using the e.g. CRISPR-CAS9 technology. Embryos are dissected and CRISPR RNA is delivered either by whole embryo electroporation or by lentiviral infection. After delivering the CRISPR RNAs, the embryos are grown ex-utero according to the protocols described herein. Upon successful growth of the embryos, the morphology is examined for potential defects caused by the gene knockouts (FIG. 27).

Optimization of E6.5 embryos electroporation was conducted with a GFP plasmid and/or Atto-labelled tracrRNA (FIG. 28). As shown in FIG. 29, Normal development together with high integration level based on the red fluorescent mark by the labelled tracrRNA was detected 16 hours following electroporation of E6.5 embryos with Atto-labelled tracrRNA.

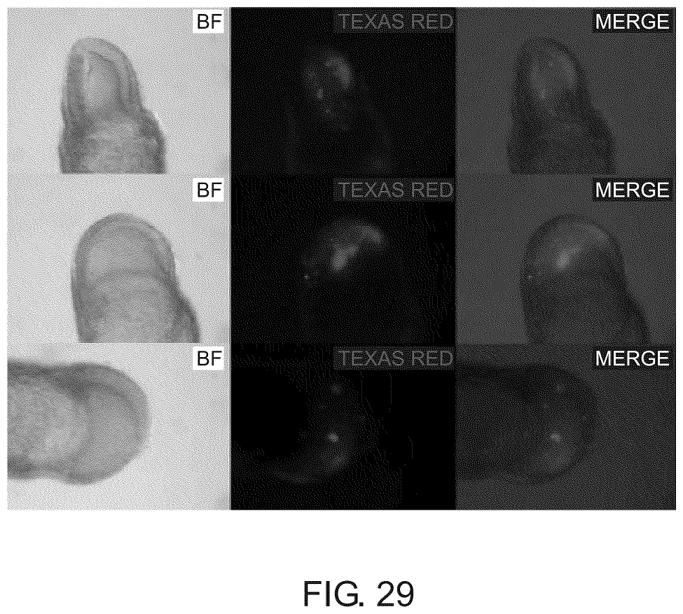

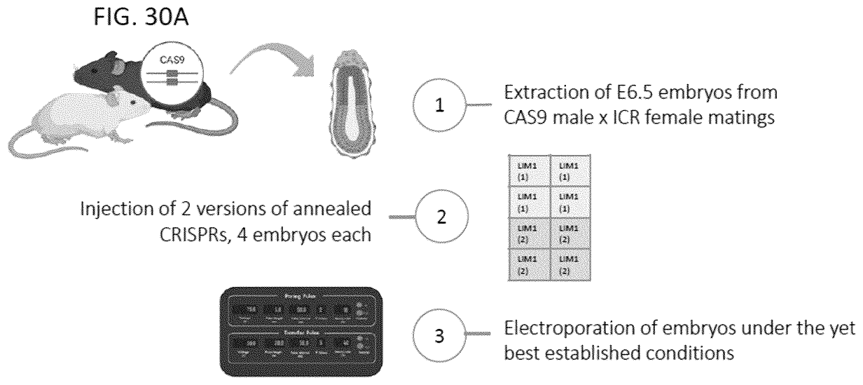

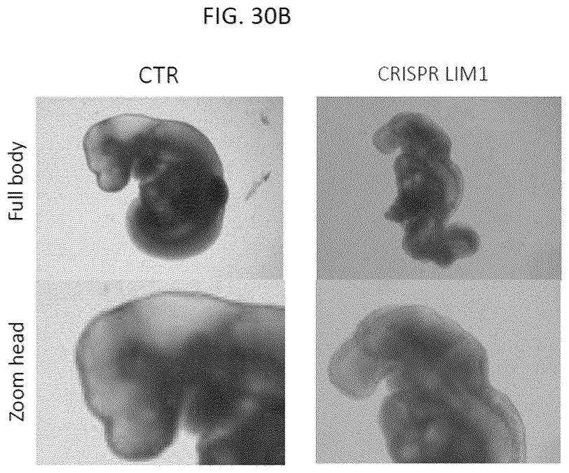

For knocking out Lim1, Pax6 or Mesp1, CRISPR sequences were annealed to tracrRNA to generate guide RNA complex (FIG. 30A). E6.5 embryos were dissected from CAS9 male xICR female matings and injected with guide CRISPR RNA LIM1 (annealed crRNA to tracrRNA) using a mouth pipet. After injection in the pro-amniotic cavity, the embryos were transferred to the electroporation chamber and electroporated under the optimized settings. Following 3 days of culture, the embryos (E9.5) showed defects in the head structure, mainly deformation and deficiency of the forebrain (FIG. 30B).

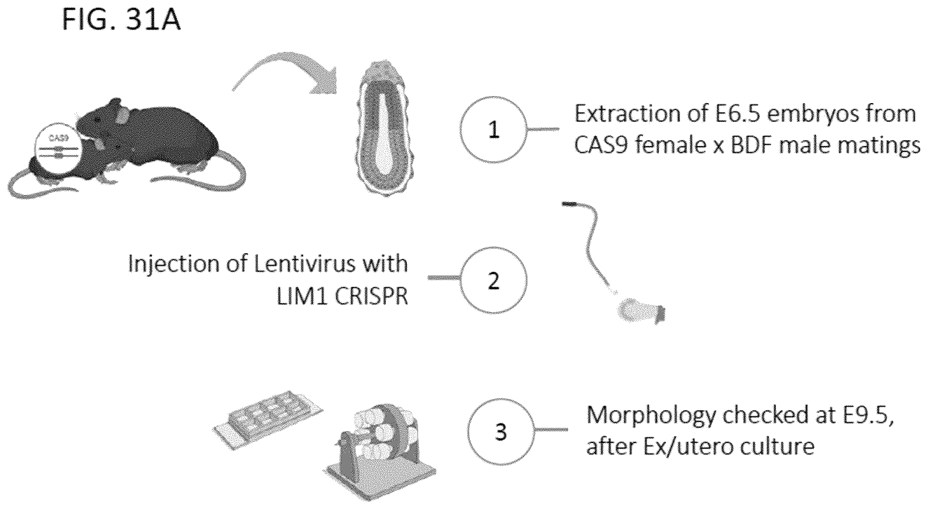

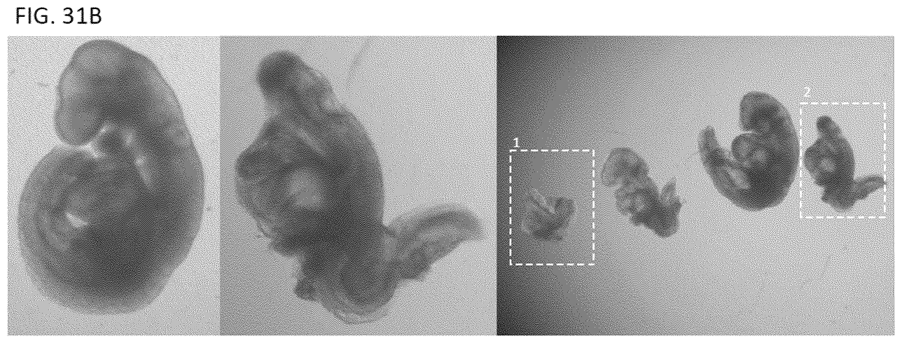

Alternatively or additionally, the CRISPR sequences were delivered via embryo lentiviral infection (FIG. 31A). E6.5 embryos were dissected from CAS9 female x BDF male matings and injected with lentivirus harboring LIM1 CRISPR RNA. Following 3 days of culture, the embryos (E9.5) showed malformation of head (FIG. 31B).

### Example 8

### Ex-Utero Culturing a Whole Rabbit Embryo

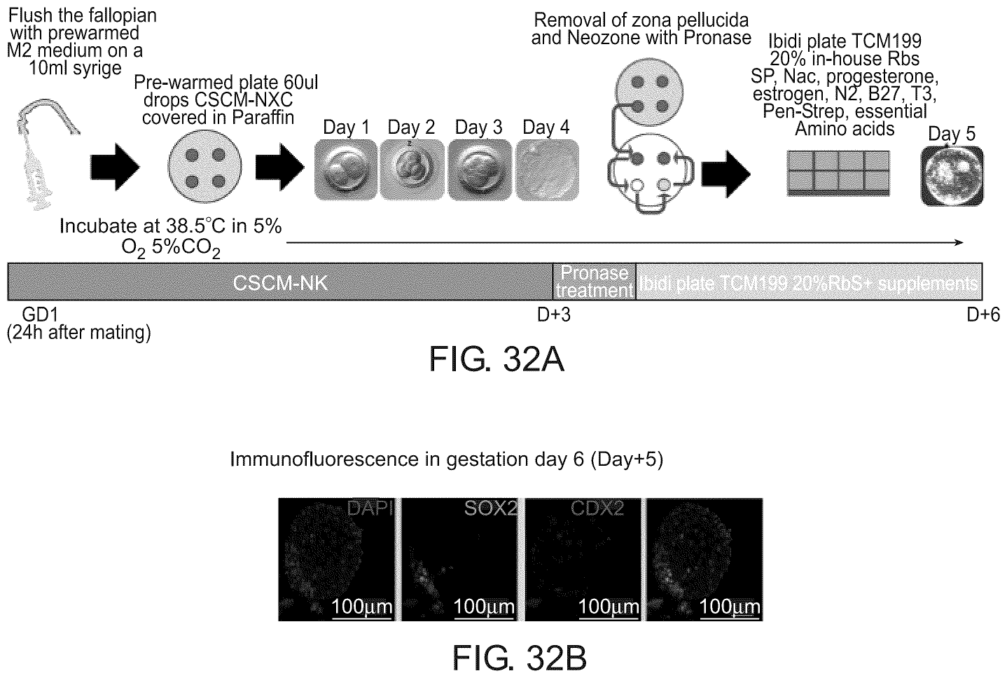

### Protocol for Ex-Utero Growing Rabbit Two Cell Embryo (GD1) Until Late Blastocyst (GD6, FIGS. 32A-B):

Time Pregnant New Zealand White rabbits were obtained from ENVIGO Israel after 24 hours of mating (termed Gestation day 1—GD1), Rabbits where Euthanized by intravenous injection of 600 mg of Pentobarbital sodium (CTS Chemical Industries), a midline abdominal incision was performed and the uterus horns were located, a clamp was inserted 1 cm before the utero-tubal junction and the fallopian tube was dissected the tissue was washed in PBS and transferred to prewarmed in-house made M2 medium. All the fat tissue was subtracted. A 10 ml syringe with a 21 g needle filled with M2 medium was inserted form the fimbria side to the lumen and 10 ml were flushed confirming the exit though the distal part. Embryos were collected using a stereoscope.

Following, embryos were transferred to 60 μl drops of CSCM-NXC (IrvineScientific) pre-warmed for 12 hours and filled with paraffin and incubated for three days at 38.5° C., 5% O₂ and 5% CO2 until blastocyst formation (which occurred in 95% of the cases).

On the 3th day of culture, on the blastocyst stage, embryos were transferred to a new drop of pre-warmed M2 medium, then to PBS followed by a 2 minutes treatment in 0.5% pronase to remove the zona pellucida and the neozona. An additional wash with M2 was performed to remove the enzyme. Afterward embryos were transferred to Ibidi u-slide plastic bottom in 250 μl of TCM199 medium supplemented with Glutamax 1×, Pencilin/Strepromycin 25 UI/ml, ITS-X 1×, Estradiol 8 nM, Progesterone 200 ng/ml, N-Acetyl-L-Cysteine 25 μM, Sodium Lactate 22%, Sodium Pyruvate 1 mM, T3 100 nM, Essential Amino acids 1× and human recombinant LIF 500 ng/ml plus 20% in-house rabbit serum. Medium was changed every 24-48 hours. Embryos are evaluated for expansion to 600 μm, integrity and maintenance of the epiblast.

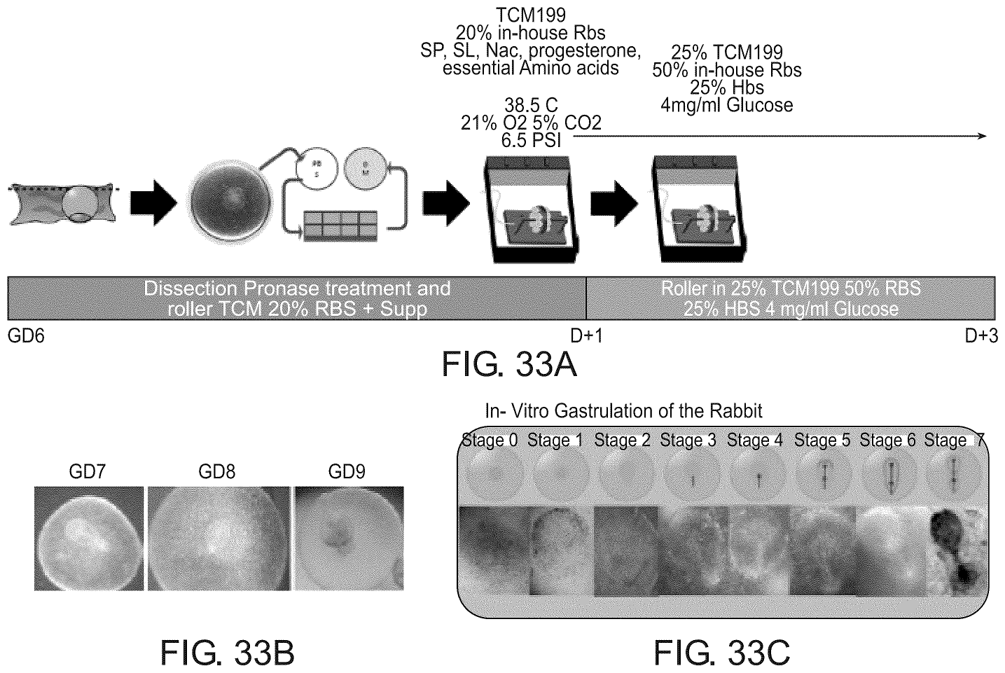

### Protocol for Ex-Utero Growing Late Blastocyst (GD6) Rabbit Embryo Until Early Organogenesis (GD9, FIGS. 33A-C):

Time Pregnant New Zealand White rabbits were obtained from ENVIGO Israel after 6 days of mating. Rabbits where Euthanized by intravenous injection of 600 mg of Pentobarbital sodium (CTS Chemical Industries). A midline abdominal incision was performed and the uterus horns were located, a cut was performed in the uterotubal junction and in the vaginal junction, both horns were washed in PBS and transferred to pre-warmed dissection medium consisting of DMEM with no phenol red (GIBCO 11880) and 10% FBS (Biological Indutires 040131A). A longitudinal incision on the mesometrial side of the uterine horns was performed and embryos were harvested using a costume shaped plastic Pasteur pipette. For Neozona Removal, embryos were transferred to a petri dish with pre-warmed PBS for washing and afterwards to a 500 μl microwell containing 0.5% pronase (Milipore 537088) for 2 minutes and then transferred back to dissection medium. Following, embryos were transferred for 24 hours to the roller culture system in TCM199 (Sigma Cat M4530) medium supplemented with Glutamax 1×, Pencilin/Strepromycin 25 UI/ml, ITS-X 1×, Estradiol 8 nM, Progesterone 200 ng/ml, N-Acetyl-L-Cysteine 25 uM, Sodium Lactate 22%, Sodium Pyruvate 1 mM, T3 100 nM and Non-Essential Amino acids 1× plus 20% in-house rabbit serum. Following 1 day of culturing, the medium was changed to 25% TCM199 supplemented with 4 mg/ml glucose (J.T Baker) plus 50% rabbit serum produced in-house and 25% human serum produces in-house. The medium was changed every 24 hours. In some embodiments, optionally, the medium further comprises knockout serum replacement (KSR) in addition to the rabbit serum and the human serum. In some embodiments, optionally, the KSR partially replaces one of either the human serum, the rabbit serum or partially replaces a quantity of both.

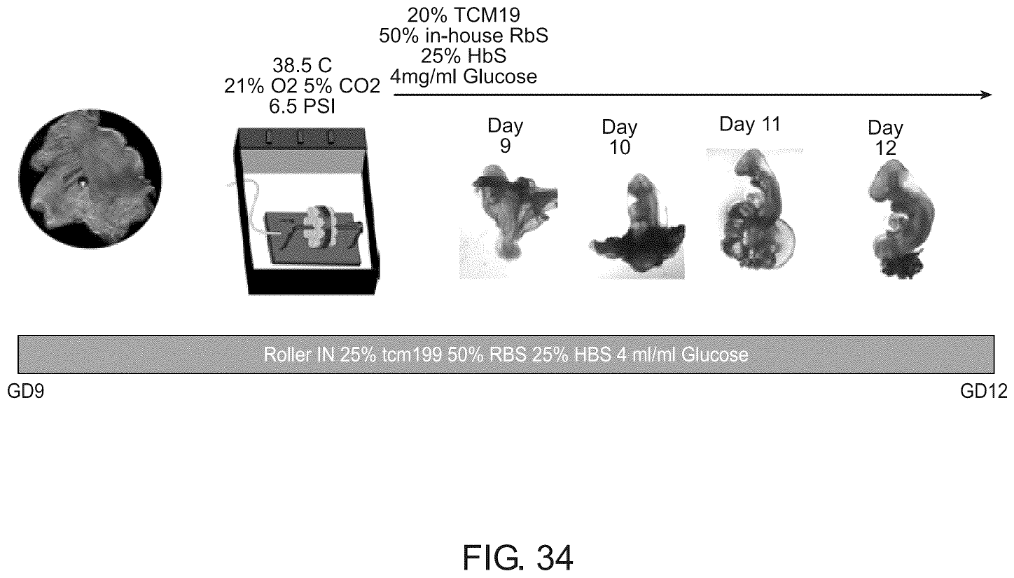

### Protocol for Ex-Utero Growing Early Organogenesis (GD9) Rabbit Embryo Until the Three Cerebral Vesicles Stage (GD12, FIG. 34):

Time Pregnant New Zealand White rabbits were obtained from ENVIGO Israel after 9 days of mating. Rabbits where euthanized by intravenous injection of 600 mg of Pentobarbital sodium (CTS Chemical Industries). A midline abdominal incision was performed and the uterus horns were located, a cut was performed in the uterotubal junction afterwards each implantation site was dissected independently. Pre-warmed dissection medium consisting of DMEM with no phenol red (GIBCO 11880) and 10% FBS (Biological Industries 040131A) was used though all the process. An insertion was performed in the anti-mesometrial site and opened flat with small dissection scissors. Embryos where dissected out using forceps.

Following, embryos were transferred to the roller culture in 25% TCM199 supplemented with 4 mg/ml glucose (J.T Baker) plus 50% rabbit serum produced in-house and 25% human serum produced in-house. The medium was changed every 24 hours. In some embodiments, optionally, the medium further comprises knockout serum replacement (KSR) in addition to the rabbit serum and the human serum. In some embodiments, optionally, the KSR partially replaces one of either the human serum, the rabbit serum or partially replaces a quantity of both.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Application

1. Bedzhov, I. & Zernicka-Goetz, M. Self-organizing properties of mouse pluripotent cells initiate morphogenesis upon implantation. Cell (2014). doi:10.1016/j.cell.2014.01.023
2. White, M. D. et al. Long-Lived Binding of Sox2 to DNA Predicts Cell Fate in the Four-Cell Mouse Embryo. Cell 165, 75-87 (2016).
3. New, D. A. T. Whole-embryo Culture and the Study of Mammalian Embryos During Organogenesis. Biol. Rev. 53, 81-122 (1978).
4. Huang, Q. et al. Intravital imaging of mouse embryos. Science (80-.). 368, 181-186 (2020).
5. Nicholas, J. S. & Rudnick, D. The Development of Rat Embryos in Tissue Culture. Proc. Natl. Acad. Sci. U.S.A. 20, 656-8 (1934).
6. New, D. A. T. & Stein, K. F. Cultivation of mouse embryos in vitro. Nature 199, 297-299 (1963).
7. New, D. A. T., Coppola, P. T. & Terry, S. Culture of explanted rat embryos in rotating tubes. J. Reprod. Fertil. (1973). doi:10.1530/jrf.0.0350135
8. New, D. A. T. Development of explanted rat embryos in circulating medium. Development 17, (1967).
9. Beddington, R. S. Induction of a second neural axis by the mouse node. Development 120, 613-620 (1994).
10. Parameswaran, M. & Tam, P. P. L. Regionalisation of cell fate and morphogenetic movement of the mesoderm during mouse gastrulation. Dev. Genet. 17, 16-28 (1995).
11. Tam, P. P. Postimplantation mouse development: whole embryo culture and micromanipulation. Int. J. Dev. Biol. 42, 895-902 (1998).
12. Cantor, J. R. et al. Physiologic Medium Rewires Cellular Metabolism and Reveals Uric Acid as an Endogenous Inhibitor of UMP Synthase. Cell 169, 258-272.e17 (2017).
13. Nagamatsu, G., Shimamoto, S., Hamazaki, N., Nishimura, Y. & Hayashi, K. Mechanical stress accompanied with nuclear rotation is involved in the dormant state of mouse oocytes. Sci. Adv. 5, eaav9960 (2019).
14. Ueda, Y. et al. Intrauterine Pressures Adjusted by Reichert's Membrane Are Crucial for Early Mouse Morphogenesis. Cell Rep. 31, 107637 (2020).
15. Downs, K. M. & Davies, T. Staging of gastrulating mouse embryos by morphological landmarks in the dissecting microscope. Development 118, 1255-1266 (1993).
16. Van Maele-Fabry, G., Delhaise, F. & Picard, J. J. Evolution of the developmental scores of sixteen morphological features in mouse embryos displaying 0 to 30 somites. Int. J. Dev. Biol. 36, 161-167 (1992).
17. Stelzer, Y. et al. Parent-of-Origin DNA Methylation Dynamics during Mouse Development. Cell Rep. 16, 3167-3180 (2016).
18. McDole, K. et al. In Toto Imaging and Reconstruction of Post-Implantation Mouse Development at the Single-Cell Level. Cell 175, 859-876.e33 (2018).
19. Ibarra-Soria, X. et al. Defining murine organogenesis at single-cell resolution reveals a role for the leukotriene pathway in regulating blood progenitor formation. Nat. Cell Biol. 20, 127-134 (2018).
20. Pijuan-Sala, B. et al. A single-cell molecular map of mouse gastrulation and early organogenesis. Nature 566, 490-495 (2019).
21. Cao, J. et al. The single-cell transcriptional landscape of mammalian organogenesis. Nature 566, 496-502 (2019).
22. Saito, T. & Nakatsuji, N. Efficient gene transfer into the embryonic mouse brain using in vivo electroporation. Dev. Biol. 240, 237-246 (2001).
23. Beronja, S., Livshits, G., Williams, S. & Fuchs, E. Rapid functional dissection of genetic networks via tissue-specific transduction and RNAi in mouse embryos. Nat. Med. 16, 821-827 (2010).
24. Morgani, S., Nichols, J. & Hadjantonakis, A. K. The many faces of Pluripotency: In vitro adaptations of a continuum of in vivo states. BMC Developmental Biology 17, (2017).
25. Kojima, Y. et al. The transcriptional and functional properties of mouse epiblast stem cells resemble the anterior primitive streak. Cell Stem Cell 14, 107-120 (2014).
26. Wu, J. et al. An alternative pluripotent state confers interspecies chimaeric competency. Nature 521, 316-321 (2015).
27. Huang, Y., Osorno, R., Tsakiridis, A. & Wilson, V. In Vivo Differentiation Potential of Epiblast Stem Cells Revealed by Chimeric Embryo Formation. Cell Rep. 2, 1571-1578 (2012).
28. Wilgenburg, B. van, Browne, C., Vowles, J. & Cowley, S. A. Efficient, Long Term Production of Monocyte-Derived Macrophages from Human Pluripotent Stem Cells under Partly-Defined and Fully-Defined Conditions. PLoS One 8, e71098 (2013).
29. Gafni, O. et al. Derivation of novel human ground state naive pluripotent stem cells. Nature 504, 282-286 (2013).
30. Harrison, S. E., Sozen, B., Christodoulou, N., Kyprianou, C. & Zernicka-Goetz, M. Assembly of embryonic and extraembryonic stem cells to mimic embryogenesis in vitro. Science 356, eaal1810 (2017).
31. Renier, N. et al. IDISCO: A simple, rapid method to immunolabel large tissue samples for volume imaging. Cell (2014). doi:10.1016/j.cell.2014.10.010
32. Stuart, T. et al. Comprehensive Integration of Single-Cell Data Resource Comprehensive Integration of Single-Cell Data. Cell 177, (2019).
33. Butler, A., Hoffman, P., Smibert, P., Papalexi, E. & Satija, R. Integrating single-cell transcriptomic data across different conditions, technologies, and species. Nat. Biotechnol. 36, 411-420 (2018).
34. Aibar, S. et al. SCENIC: Single-cell regulatory network inference and clustering. Nat. Methods 14, 1083-1086 (2017).
35. Ritchie, M. E. et al. Limma powers differential expression analyses for RNA-sequencing and microarray studies. Nucleic Acids Res. 43, e47 (2015).
36. Bedzhov I, Leung C Y, Bialecka M, Zernicka-Goetz M. In vitro culture of mouse blastocysts beyond the implantation stages. Nat Protoc. 2014 December; 9(12):2732-9. doi: 10.1038/nprot.2014.186.

What is claimed is:

1. A fetal incubation system for at least one embryo, comprising:
a. a gas controller, configured for providing a plurality of gases to at least one incubator;
b. at least one incubator comprising a rotating module inside of said at least one incubator; said rotating module comprising one or more vials comprising said at least one embryo;
wherein said system comprises one or more buffers for said plurality of gases being provided to said rotating module inside of said at least one incubator;
wherein said rotating module is configured for incubating said at least one embryo in said one or more vials whilst performing one or more of:
i. rotation of said one or more vials; and
ii. delivering a combination of gases from said plurality of gases to within of said one or more vials.

2. The fetal incubation system according to claim 1, wherein one of said one or more buffers is a gas mixing box for mixing said plurality of gases before being provided to said at least one incubator.

3. The fetal incubation system according to claim 1, wherein said gas controller comprises one or more of:
a. one or more specific gas controllers for individually control flow of a specific gas from said plurality of gases; and
b. one or more electric valves for allowing flowing of specific gases from said plurality of gases.

4. The fetal incubation system according to claim 1, wherein said gas controller comprises one or more of:
a. a vacuum pump for extracting mixed gases from said gas mixing box; and
b. a pressure pump in connection with said vacuum pump for providing said mixed gases to said system at hyperbaric pressures of from about 0.1 psi to about 20 psi.

5. The fetal incubation system according to claim 1, wherein said gas mixing box comprises one or more gas sensors.

6. The fetal incubation system according to claim 5, wherein said one or more specific gas controllers control activation and deactivation of said one or more electric valves according to said information received by said one or more gas sensors.

7. The fetal incubation system according to claim 1, further comprising a humidifier, which also functions as one of said buffers for said plurality of gases.

8. The fetal incubation system according to claim 7, wherein said rotational module comprises a rotational drum comprising said one or more vials; said rotational drum connected to said humidifier.

9. The fetal incubation system according to claim 1, wherein said incubator comprises an outlet bottle for gases.

10. The fetal incubation system according to claim 9, wherein said outlet bottle for gases functions as one of said buffers for said plurality of gases.

11. The fetal incubation system according to claim 1, wherein said one or more buffers are configured to maintain a determined concentration of said plurality of gases and a determined hyperbaric level substantially constant.

12. The fetal incubation system according to claim 1, wherein said at least one embryo is at least one synthetic embryo.

13. A method of incubating at least one embryo in an incubator, comprising:
a. flowing mixed gases at a determined concentration into a rotating module comprising one or more vials including said at least one embryo, said rotating module located inside said incubator;
b. flowing said mixed gases at a determined hyperbaric level within said one or more vials;
c. maintaining said determined concentration and said determined hyperbaric level substantially constant whilst rotating said one or more vials.

14. The method according to claim 13, wherein achieving said mixed gases at said determined concentration, comprises:
a. setting desired concentrations of each individual gas of said mixed gases;
b. flowing said individual gases into a gas mixing box;
c. sensing when each of said individual gases reaches said desired concentration;
d. mixing said gases inside said gas mixing box.

15. The method according to claim 13, wherein achieving said determined hyperbaric level, comprises:
a. setting a desired hyperbaric level;
b. allowing access of said mixed gases to a pressure pump until said hyperbaric level is reached.

16. The method according to claim 13, wherein said maintaining comprises providing a plurality of sensors to said gas mixing box for monitoring said concentrations of said each individual gas.

17. The method according to claim 13, wherein said maintaining comprises providing pressure stabilizers/buffers in said incubator.

18. The method according to claim 17, wherein said providing pressure stabilizers/buffers in said incubator comprises providing said pressure stabilizers/buffers before and/or after said rotational module.

19. The method according to claim 13, wherein said incubating is effected using the fetal incubation system according to claim 1.

20. The method according to claim 13, wherein said at least one embryo is at least one synthetic embryo.
